SEQUENCE LISTING (121 sequences)

SEQ ID NO: 1 - protein, 137 aa, Homo sapiens
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 2 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - protein, 111 aa, Homo sapiens
APLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLA

SEQ ID NO: 4 - protein, 247 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 5 - protein, 111 aa, Homo sapiens
APLKIQAYFNETADLPCQFANSQNQSLSELVVFWQDQENLVLNEVYLGKEKFDSVASKYMGRTSFDSDSWTLRLHNLQIKDKGLYQCIIHHKKPTGMIRIHQMNSELSVLA

SEQ ID NO: 6 - protein, 109 aa, Artificial Sequence
  Synthetic construct clone number 900
LKIQAYFNETADLPCQFANSQNQSLSELVVFWQDQENLVLNEVYLGKEKFDSVDSKYMGRTSFDSDSWTLRLHNLQIKDKGIYQCVIHHKKPSGLVKIHEMNSELSVLA

SEQ ID NO: 7 - protein, 109 aa, Artificial Sequence
  Synthetic construct clone number 901
LKIQAYFNETADLPCQFANSQNLTLSELVVFWQDQENLVLNEVYLGKEKFDSVHSKYMGRTSFDSDSWTLRLHNLQIKDKGIYQCVIHHKKPTGMIKIHEMNSELSVLT

SEQ ID NO: 8 - protein, 109 aa, Artificial Sequence
  Synthetic construct clone number 904
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLA

SEQ ID NO: 9 - protein, 109 aa, Artificial Sequence
  Synthetic construct clone number 906
LKIQAYINETADLPCQFANSQNLSLSELVVFWQDQENLVLNEVYLGKERFDSVDSKYMGRTSFDSDSWTLRLHNLQIKDKGFYQCIIHHKKPTGLVKIHEMNSELSVLA

SEQ ID NO: 10 - protein, 109 aa, Artificial Sequence
  Synthetic construct clone number 907
LKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIKIHEMNSELSVLA

SEQ ID NO: 11 - protein, 109 aa, Artificial Sequence
  Synthetic construct clone number 908
LKIQAYFNETADLPCQFANSQNQSLSELVVFWQDQENLVLNEVYLGKEKFDSVHSKYMGRTSFDSDSWTLRLHNLQIKDKGIYQCIIHHKKPTGMVKIHEMNSELSVLA

SEQ ID NO: 12 - protein, 109 aa, Artificial Sequence
  Synthetic construct clone number 910
LKIQAYFNETADLPCQFANSQNQSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 13 - protein, 109 aa, Artificial Sequence
  Synthetic construct clone number 915
LKIQAYFNETADLPCQFANSQNQSLSELVVFWQDQENLILNEVYLGKEKFDSVDSKYMGRTSFDSDSWTLRLHNLQIKDKGFYQCIIHHKKPSGLIKIHQMDSELSVLA

SEQ ID NO: 14 - protein, 109 aa, Artificial Sequence
  Synthetic construct clone number 938
LKIQAYFNETADLPCQFANSQNQSLSELVVFWQDQENLILNEVYLGKEKFDSVHSKYMGRTSFDSDSWTLRLHNLQIKDKGIYQCIIHHKKPTGMVKIHQMNSELSVLA

SEQ ID NO: 15 - protein, 111 aa, Artificial Sequence
  Synthetic construct clone number 1038
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMVKIHEMNSELSVLA

SEQ ID NO: 16 - protein, 111 aa, Artificial Sequence
  Synthetic construct clone number 1039
APLKIQAYFNETADLPCQFANSQNLSLSELVVFWQDQENLVLNEVYLGKEKFDSVSSKYMGRTSFDSDSWTLRLHNLQIKDKGIYQCIIHHKKPSGMVKIHQMDSELSVLA

SEQ ID NO: 17 - protein, 111 aa, Artificial Sequence
  Synthetic construct clone number 1040
APLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 18 - protein, 111 aa, Artificial Sequence
  Synthetic construct clone number 1041
APLKIQAYLNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 19 - protein, 111 aa, Artificial Sequence
  Synthetic construct clone number 1042
APLKIQAYFNETADLPCQFANSQNLSLSELVVFWQDQENLVLNEVYLGKEIFDSVSSKYMGRTSFDSDSWTLRLHNLQIKDKGIYQCIIHHKKPSGMVKIHQMDSELSVLA

SEQ ID NO: 20 - protein, 111 aa, Artificial Sequence
  Synthetic construct clone number 1043
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGMIKIHEMNSELSVLA

SEQ ID NO: 21 - protein, 111 aa, Artificial Sequence
  Synthetic construct clone number 1044
APLKIQAYFNETADLPCQFANSQNLTLSELVVFWQDQENLVLNEVYLGKEKFDSVSSKYMGRTSFDSDSWTLRLHNLQIKDKGIYQCIIHHKKPTGMIKIHEMSSELSVLA

SEQ ID NO: 22 - protein, 111 aa, Artificial Sequence
  Synthetic construct clone number 1045
APLKIQAYFNETADLPCQFANSQNLTLSELVVFWQDQENLVLNEVYLGKEKFDSVDSKYMGRTSFDSDSWTLRLHNLQIKDKGLYQCIIHHKKPTGLVKIHEMNSELSVLA

SEQ ID NO: 23 - protein, 111 aa, Artificial Sequence
  Synthetic construct clone number 1046
APLKIQAYFNETADLPCQFANSQNQSLSELVVFWQDQENLVLNEVYLGKEKFDSVDSKYMGRTSFDSDSWTLRLHNLQIEDKGIYQCIIHHKKPSGMVKIHQMDSELSVLA

SEQ ID NO: 24 - protein, 111 aa, Artificial Sequence
  Synthetic construct clone number 1047
APLKIQAYFNETADLPCQFANSQNLSLSELVVFWQDQENLVLNEVYLGKEKFDSVDSKYMGRTSFDSDSWTLRLHNLQIKDKGIYQCIIHHKKPTGLVKIHEMNSELSVLA

SEQ ID NO: 25 - DNA, 327 bp, Artificial Sequence
  Synthetic construct of clone number 900
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcgtgatccaccataagaagccgagcggtctggtgaagattcacgagatgaactccgagttgtctgtcctggcg

SEQ ID NO: 26 - DNA, 327 bp, Artificial Sequence
  Synthetic construct of clone number 901
ctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcgtgatccaccataagaagccgacgggtatgattaagattcacgagatgaactccgagttgtctgtcctgacc

SEQ ID NO: 27 - DNA, 327 bp, Artificial Sequence
  Synthetic construct of clone number 904
ctcaaaatccaagcgtacttcaacgaaactgcagacttaccgtgtcagtttgccaattcgcagaatcaaagcctgagcgaactgatcgttttctggcaggatcaggagaacctggttctgaacgaagtctatctgggcaaagagcggttcgacgccgtggacagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 28 - DNA, 327 bp, Artificial Sequence
  Synthetic construct of clone number 906
ctcaaaatccaagcgtacatcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtttctaccagtgcattatccaccataagaagccgacgggtctggtgaagattcacgagatgaactccgagttgtctgtcctggcg

SEQ ID NO: 29 - DNA, 327 bp, Artificial Sequence
  Synthetic construct of clone number 907
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtctgtaccagtgcattatccaccataagaagccgacgggtatgattaagattcacgagatgaactccgagttgtctgtcctggcg

SEQ ID NO: 30 - DNA, 327 bp, Artificial Sequence
  Synthetic construct of clone number 908
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtgcatagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 31 - DNA, 327 bp, Artificial Sequence
  Synthetic construct of clone number 910
ctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 32 - DNA, 327 bp, Artificial Sequence
  Synthetic construct for clone number 915
ctcaaaatccaagcgtacttcaacgaaactgcagacttaccgtgtcagtttgccaattcgcagaatcaaagcctgagcgaactggtggttttctggcaggatcaggagaacctgatcctgaacgaagtctatctgggcaaagagaaattcgacagcgtggacagcaagtatatgggccgcaccagctttgatagcgacagctggaccctgcgtctgcacaatctgcaaatcaaagataagggtttctaccagtgcattatccaccataagaagccgagcggtctgattaagattcaccaaatggactccgagttgtctgtcctggcg

SEQ ID NO: 33 - DNA, 327 bp, Artificial Sequence
  Synthetic construct of clone number 938
ctcaaaatccaagcgtacttcaacgaaactgcagacttaccgtgtcagtttgccaattcgcagaatctgagcctgagcgaactggtggttttctggcaggatcaggagaacctgatcctgaacgaagtctatctgggcaaagagcggttcgacagcgtgcatagcaagtatatgggccgcaccagctttgatagcgacagctggaccctgcgtctgcacaatctgcaaatcaaagataagggtctgtaccagtgcattatccaccataagaagccgagcggtatggtgaagattcacgagatgaactccgagttgtctgtcctggcg

SEQ ID NO: 34 - DNA, 333 bp, Artificial Sequence
  Synthetic construct of clone number 1038
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtatggtgaagattcacgagatgaactccgagttgtctgtcctggcg

SEQ ID NO: 35 - DNA, 333 bp, Artificial Sequence
  Synthetic construct of clone number 1039
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtgagtagcaagtatatgggccgcaccagctttgatagcgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 36 - DNA, 333 bp, Artificial Sequence
  Synthetic construct of clone number 1040
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 37 - DNA, 333 bp, Artificial Sequence
  Synthetic construct of clone number 1041
gcccccctcaaaatccaagcgtacctcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 38 - DNA, 333 bp, Artificial Sequence
  Synthetic construct of clone number 1042
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagattttcgacagcgtgagtagcaagtatatgggccgcaccagctttgatagtgacagctggaccctgcgtctgcacaatctgcaaatcaaagataagggtatctaccagtgcattatccaccataagaagccgagcggtatggtgaagattcaccaaatggactccgagttgtctgtcctggcg

SEQ ID NO: 39 - DNA, 333 bp, Artificial Sequence
  Synthetic construct of clone number 1043
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggatagcaagtatatgggccgcaccagctttgatagcgacagctggaccctgcgtctgcacaatctgcaaatcaaagataagggtatctaccagtgcattatccaccataagaagccgacgggtatgattaagattcacgagatgaactccgagttgtctgtcctggcg

SEQ ID NO: 40 - DNA, 333 bp, Artificial Sequence
  Synthetic construct of clone number 1044
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtgtctagcaagtatatgggccgcaccagctttgatagcgacagctggaccctgcgtctgcacaatctgcaaatcaaagataagggtatctaccagtgcattatccaccataagaagccgacgggtatgattaagattcacgagatgagctccgagttgtctgtcctggcg

SEQ ID NO: 41 - DNA, 333 bp, Artificial Sequence
  Synthetic construct of clone number 1045
gcccccctcaaaatccaagcgtacttcaacgaaactgcagacttaccgtgtcagtttgccaattcgcagaatctgaccctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtctgtaccagtgcattatccaccataagaagccgacgggtctggtgaagattcacgagatgaactccgagttgtctgtcctggcg

SEQ ID NO: 42 - DNA, 333 bp, Artificial Sequence
  Synthetic construct of clone number 1046
gcccccctcaaaatccaagcgtacttcaacgaaactgcagacttaccgtgtcagtttgccaattcgcagaatcaaagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcgaagataagggtatctaccagtgcattatccaccataagaagccgagcggtatggtgaagattcaccaaatggactccgagttgtctgtcctggcg

SEQ ID NO: 43 - DNA, 333 bp, Artificial Sequence
  Synthetic construct of clone number 1047
gcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagaaattcgacagcgtggacagcaagtatatgggccgcaccagctttgatagcgacagctggaccctgcgtctgcacaatctgcaaatcaaagataagggtatctaccagtgcattatccaccataagaagccgacgggtctggtgaagattcacgagatgaactccgagttgtctgtcctggcg

SEQ ID NO: 44 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 45 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 46 - protein, 11 aa, Artificial Sequence
  Linker sequence
SGGGGSGGGGS

SEQ ID NO: 47 - protein, 13 aa, Artificial Sequence
  Linker sequence
SGGGGSGGGGSAP

SEQ ID NO: 48 - protein, 5 aa, Artificial Sequence
  Linker sequence
NFSQP

SEQ ID NO: 49 - protein, 5 aa, Artificial Sequence
  Linker sequence
KRTVA

SEQ ID NO: 50 - protein, 8 aa, Artificial Sequence
  Linker sequence
GFTFSDYY

SEQ ID NO: 51 - protein, 8 aa, Artificial Sequence
  Linker sequence
ISSNGIYI

SEQ ID NO: 52 - protein, 15 aa, Artificial Sequence
  Linker sequence
ARAPVDYSNPSGMDV

SEQ ID NO: 53 - protein, 8 aa, Artificial Sequence
  Linker sequence
SSNIGSNT

SEQ ID NO: 54 - protein, 3 aa, Artificial Sequence
  Linker sequence
RNN

SEQ ID NO: 55 - protein, 9 aa, Artificial Sequence
  Linker sequence
AAWDDSLSG

SEQ ID NO: 56 - protein, 338 aa, Homo sapiens
QSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGWRFGGGTKLTVLGGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSLKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLA

SEQ ID NO: 57 - protein, 340 aa, Homo sapiens
QSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGWRFGGGTKLTVLGGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLA

SEQ ID NO: 58 - protein, 566 aa, Homo sapiens
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLANFSQPEVQLLESGGGLVQPGGSLRLSCAASGFTFSDYYMSWVRQAPGKGLEWISSISSNGIYIYYADSLKGRFTISRDNSKNTLYLQMNSLRAEDTAVYYCARAPVDYSNPSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 332 aa, Homo sapiens
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLAKRTVAQSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGWRFGGGTKLTVLGGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 332 aa, Homo sapiens
LKIQAYFNETADLPCQFANSQNQSLSELIVFWQDQENLVLNEVYLGKERFDAVDSKYMGRTSFDSDSWTLRLHNLQIKDKGIYQCIIHHKKPSGMVKIHQMDSELSVLANFSQPQSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGWRFGGGTKLTVLGGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 452 aa, Homo sapiens
EVQLLESGGGLVQPGGSLRLSCAASGFTFSDYYMSWVRQAPGKGLEWISSISSNGIYIYYADSLKGRFTISRDNSKNTLYLQMNSLRAEDTAVYYCARAPVDYSNPSGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 218 aa, Homo sapiens
QSVLTQPPSASGTPGQRVTISCSGSSSNIGSNTVNWYQQLPGTAPKLLIYRNNQRPSGVPDRFSGSKSGTSASLAISGLRSEDEADYYCAAWDDSLSGWRFGGGTKLTVLGGTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - DNA, 4388 bp, Homo sapiens
gaggtgcagctgctggagtccggaggaggcctggtgcagcccggcggcagcctgagactgagctgtgccgccagcggattcaccttctccgactactatatgagctgggtgaggcaggccccaggcaagggcctggagtggatttcctccatcagcagcaatgggatctacatttactacgccgacagcctgaagggcaggttcacaatcagcagggacaactctaagaacacactgtacctgcagatgaactccctgcgcgccgaggacaccgccgtgtattactgtgccagggcccccgtggactactctaatcccagcggcatggacgtgtggggccagggcaccctggtgacagtgagctcaggtgagtcgtacgctagcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagaggtgcagctgctggagtccggaggaggcctggtgcagcccggcggcagcctgagactgagctgtgccgccagcggattcaccttctccgactactatatgagctgggtgaggcaggccccaggcaagggcctggagtggatttcctccatcagcagcaatgggatctacatttactacgccgacagcctgaagggcaggttcacaatcagcagggacaactctaagaacacactgtacctgcagatgaactccctgcgcgccgaggacaccgccgtgtattactgtgccagggcccccgtggactactctaatcccagcggcatggacgtgtggggccagggcaccctggtgacagtgagctcaggtgagtcgtacgctagcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 64 - DNA, 1023 bp, Homo sapiens
cagtccgtgctgacccagccaccctccgccagcggcacccctggccagcgggtgaccatctcttgtagcggcagcagctccaacatcggaagcaataccgtgaattggtaccagcagctgcccggcaccgcccccaagctgctgatctaccgcaataaccagaggccctccggcgtgcccgacaggttcagcggcagcaagtctggcacctccgcctctctggccattagcggactgcggagcgaggacgaggccgattactactgcgccgcctgggacgactccctgtccgggtggcgctttggaggcggcacaaagctgaccgtgctgggaggtgagtagaacgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 65 - DNA, 1383 bp, Homo sapiens
cagtccgtgctgacccagccaccctccgccagcggcacccctggccagcgggtgaccatctcttgtagcggcagcagctccaacatcggaagcaataccgtgaattggtaccagcagctgcccggcaccgcccccaagctgctgatctaccgcaataaccagaggccctccggcgtgcccgacaggttcagcggcagcaagtctggcacctccgcctctctggccattagcggactgcggagcgaggacgaggccgattactactgcgccgcctgggacgactccctgtccgggtggcgctttggaggcggcacaaagctgaccgtgctgggaggtgagtagaacgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcctcaagattcaggcgtactttaacgaaacagcggacctcccgtgccaattcgccaatagccagaatcaatcgctcagtgagctcatcgtgttttggcaagatcaggaaaaccttgtcttgaatgaggtctatctgggtaaagagagattcgacgccgtagattccaagtacatggggaggacttcgttcgattcagattcgtggacgctgagattgcataatctccaaatcaaagacaaagggatctaccagtgcatcattcaccacaaaaagcctagcggaatggtgaagatccatcagatggacagcgaattgtcagttttggcc

SEQ ID NO: 66 - DNA, 1389 bp, Homo sapiens
cagtccgtgctgacccagccaccctccgccagcggcacccctggccagcgggtgaccatctcttgtagcggcagcagctccaacatcggaagcaataccgtgaattggtaccagcagctgcccggcaccgcccccaagctgctgatctaccgcaataaccagaggccctccggcgtgcccgacaggttcagcggcagcaagtctggcacctccgcctctctggccattagcggactgcggagcgaggacgaggccgattactactgcgccgcctgggacgactccctgtccgggtggcgctttggaggcggcacaaagctgaccgtgctgggaggtgagtagaacgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaagattcaggcgtactttaacgaaacagcggacctcccgtgccaattcgccaatagccagaatcaatcgctcagtgagctcatcgtgttttggcaagatcaggaaaaccttgtcttgaatgaggtctatctgggtaaagagagattcgacgccgtagattccaagtacatggggaggacttcgttcgattcagattcgtggacgctgagattgcataatctccaaatcaaagacaaagggatctaccagtgcatcattcaccacaaaaagcctagcggaatggtgaagatccatcagatggacagcgaattgtcagttttggcc

SEQ ID NO: 67 - DNA, 2537 bp, Homo sapiens
ctcaagattcaggcgtactttaacgaaacagcggacctcccgtgccaattcgccaatagccagaatcaatcgctcagtgagctcatcgtgttttggcaagatcaggaaaaccttgtcttgaatgaggtctatctgggtaaagagagattcgacgccgtagattccaagtacatggggaggacttcgttcgattcagattcgtggacgctgagattgcataatctccaaatcaaagacaaagggatctaccagtgcatcattcaccacaaaaagcctagcggaatggtgaagatccatcagatggacagcgaattgtcagttttggccaacttcagccagcccgaggtgcagctgctggagtccggaggaggcctggtgcagcccggcggcagcctgagactgagctgtgccgccagcggattcaccttctccgactactatatgagctgggtgaggcaggccccaggcaagggcctggagtggatttcctccatcagcagcaatgggatctacatttactacgccgacagcctgaagggcaggttcacaatcagcagggacaactctaagaacacactgtacctgcagatgaactccctgcgcgccgaggacaccgccgtgtattactgtgccagggcccccgtggactactctaatcccagcggcatggacgtgtggggccagggcaccctggtgacagtgagctcaggtgagtcgtacgctagcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgag

SEQ ID NO: 68 - DNA, 1365 bp, Homo sapiens
ctcaagattcaggcgtactttaacgaaacagcggacctcccgtgccaattcgccaatagccagaatcaatcgctcagtgagctcatcgtgttttggcaagatcaggaaaaccttgtcttgaatgaggtctatctgggtaaagagagattcgacgccgtagattccaagtacatggggaggacttcgttcgattcagattcgtggacgctgagattgcataatctccaaatcaaagacaaagggatctaccagtgcatcattcaccacaaaaagcctagcggaatggtgaagatccatcagatggacagcgaattgtcagttttggccaagagaaccgtggcccagtccgtgctgacccagccaccctccgccagcggcacccctggccagcgggtgaccatctcttgtagcggcagcagctccaacatcggaagcaataccgtgaattggtaccagcagctgcccggcaccgcccccaagctgctgatctaccgcaataaccagaggccctccggcgtgcccgacaggttcagcggcagcaagtctggcacctccgcctctctggccattagcggactgcggagcgaggacgaggccgattactactgcgccgcctgggacgactccctgtccgggtggcgctttggaggcggcacaaagctgaccgtgctgggaggtgagtagaacgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 69 - DNA, 1365 bp, Homo sapiens
ctcaagattcaggcgtactttaacgaaacagcggacctcccgtgccaattcgccaatagccagaatcaatcgctcagtgagctcatcgtgttttggcaagatcaggaaaaccttgtcttgaatgaggtctatctgggtaaagagagattcgacgccgtagattccaagtacatggggaggacttcgttcgattcagattcgtggacgctgagattgcataatctccaaatcaaagacaaagggatctaccagtgcatcattcaccacaaaaagcctagcggaatggtgaagatccatcagatggacagcgaattgtcagttttggccaacttcagccagccccagtccgtgctgacccagccaccctccgccagcggcacccctggccagcgggtgaccatctcttgtagcggcagcagctccaacatcggaagcaataccgtgaattggtaccagcagctgcccggcaccgcccccaagctgctgatctaccgcaataaccagaggccctccggcgtgcccgacaggttcagcggcagcaagtctggcacctccgcctctctggccattagcggactgcggagcgaggacgaggccgattactactgcgccgcctgggacgactccctgtccgggtggcgctttggaggcggcacaaagctgaccgtgctgggaggtgagtagaacgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 70 - protein, 446 aa, Artificial Sequence
  antibody H chain 1107
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRVWGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, Artificial Sequence
  antibody L chain 1145
DIQMTQSPSSLSASVGDRVTITCRASQSISSYLNWYQQKPGKAPKLLIYAASSLQSGVPSRFSGSGSGTDFTLTISSLQPEDFATYYCQQYGVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 446 aa, Artificial Sequence
  antibody H chain 1136
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYVFGIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 215 aa, Artificial Sequence
  antibody L chain 1143
DIQMTQSPSSLSASVGDRVTITCRASQSISSYLNWYQQKPGKAPKLLIYAASSLQSGVPSRFSGSGSGTDFTLTISSLQPEDFATYYCQQAYYAGLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 447 aa, Artificial Sequence
  antibody H chain 1132
EVQLLESGGGLVQPGGSLRLSCAASGFTFSSYAMSWVRQAPGKGLEWVSGIGSYGGGTYYADSVKGRFTISRDNSKNTLYLQMNSLRAEDTAVYYCARYVNFGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 214 aa, Artificial Sequence
  antibody L chain 1139
DIQMTQSPSSLSASVGDRVTITCRASQSISSYLNWYQQKPGKAPKLLIYAASSLQSGVPSRFSGSGSGTDFTLTISSLQPEDFATYYCQQYGRNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 449 aa, Artificial Sequence
  antibody H chain 1140
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGPVYSSVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, Artificial Sequence
  antibody L chain 1141
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 447 aa, Artificial Sequence
  antibody H chain 1150
EVQLLESGGGLVQPGGSLRLSCAASGFTFSSYAMSWVRQAPGKGLEWVSGIGGSSSYTSYADSVKGRFTISRDNSKNTLYLQMNSLRAEDTAVYYCARYYSYHMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 214 aa, Artificial Sequence
  antibody L chain 1152
DIQMTQSPSSLSASVGDHVTITCRASQSISSYLNWYQQKPGKAPKLLIYAASSLQSGVPSRFSGSGSGTDFTLTISSLQPEDFATYYCQQYGSAPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 449 aa, Artificial Sequence
  antibody H chain 1134
EVQLLESGGGLVQPGGSLRLSCAASGFTFSSYAMSWVRQAPGKGLEWVSSIYSGGGGTSYADSVKGRFTISRDNSKNTLYLQMNSLRAEDTAVYYCARGPAYSSFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 214 aa, Artificial Sequence; identical to SEQ ID NO: 77
  antibody L chain 1141

SEQ ID NO: 82 - protein, 446 aa, Artificial Sequence
  antibody H chain 1146
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARRVFGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 214 aa, Artificial Sequence
  antibody L chain 1147
DIQMTQSPSSLSASVGDRVTITCRASQSISSYLNWYQQKPGKAPKLLIYAASSLQSGVPSRFSGSGSGTDFTLTISSLQPEDFATYYCQQYYYYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 449 aa, Artificial Sequence
  antibody H chain 1142
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGPAYSTVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 214 aa, Artificial Sequence; identical to SEQ ID NO: 77
  antibody L chain 1135

SEQ ID NO: 86 - protein, 446 aa, Artificial Sequence
  antibody H chain 1148
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARAVFGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 214 aa, Artificial Sequence
  antibody L chain 1149
DIQMTQSPSSLSASVGDRVTITCRASQSISSYLNWYQQKPGKAPKLLIYAASSLQSGVPSRFSGSGSGTDFTLTISSLQPEDFATYYCQQAYYFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 449 aa, Artificial Sequence
  antibody H chain 1138
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGFVYSSYIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 214 aa, Artificial Sequence; identical to SEQ ID NO: 77
  antibody L chain 1135

SEQ ID NO: 90 - DNA, 348 bp, Artificial Sequence
  antibody H chain 1107 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgccgtgtttggggttttgactattggggccagggaaccctggtcaccgtctcctca

SEQ ID NO: 91 - DNA, 949 bp, Artificial Sequence
  antibody L chain 1145
ccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtgtttacccgttcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 92 - DNA, 348 bp, Artificial Sequence
  antibody H chain 1136 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgctacgttttcggtattgactattggggccagggaaccctggtcaccgtctcctca

SEQ ID NO: 93 - DNA, 1010 bp, Artificial Sequence
  antibody L chain 1143
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacaggcttactacgctggtctgttcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 94 - DNA, 351 bp, Artificial Sequence
  antibody H chain 1132 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcaggtattggttcttacggtggtggtacatactatgcagactccgtgaagggccggttcaccatctcccgtgacaattccaagaacacgctgtatctgcaaatgaacagcctgcgtgccgaggacacggctgtatattattgtgcgcgctacgttaacttcggtatggactattggggccagggaaccctggtcaccgtctcctca

SEQ ID NO: 95 - DNA, 1007 bp, Artificial Sequence
  antibdoy L chain 1139
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtcgtaacccgcccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 96 - DNA, 357 bp, Artificial Sequence
  antibody H chain 1140 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgcggtccggtttactcttctgtttttgactattggggccagggaaccctggtcaccgtctcctca

SEQ ID NO: 97 - DNA, 1007 bp, Artificial Sequence
  antibody L chain 1141
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 98 - DNA, 351 bp, Artificial Sequence
  antibody H chain 1150 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcaggtattggtggttcttcttcttacacatcttatgcagactccgtgaagggccggttcaccatctcccgtgacaattccaagaacacgctgtatctgcaaatgaacagcctgcgtgccgaggacacggctgtatattattgtgcgcgctactactcttaccatatggactattggggccagggaaccctggtcaccgtctcctca

SEQ ID NO: 99 - DNA, 1007 bp, Artificial Sequence
  antibody L chain 1152
gacatccagatgacccagtctccatcctccctgagcgcatctgtaggagaccacgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctgctccgcccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 100 - DNA, 357 bp, Artificial Sequence
  antibody H chain 1134 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcatctatttactctggtggtggtggtacatcttatgcagactccgtgaagggccggttcaccatctcccgtgacaattccaagaacacgctgtatctgcaaatgaacagcctgcgtgccgaggacacggctgtatattattgtgcgcgcggtccggcttactcttctttctttgactattggggccagggaaccctggtcaccgtctcctca

SEQ ID NO: 101 - DNA, 1007 bp, Artificial Sequence; identical to SEQ ID NO: 97
  antibody L chain 1141

SEQ ID NO: 102 - DNA, 348 bp, Artificial Sequence
  antibody H chain 1146 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgccgtgttttcggttttgactattggggccagggaaccctggtcaccgtctcctca

SEQ ID NO: 103 - DNA, 321 bp, Artificial Sequence
  antibody L chain 1147 V region
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtactactactacccgttcacttttggccaggggaccaagctggagatcaaa

SEQ ID NO: 104 - DNA, 357 bp, Artificial Sequence
  antibody H chain 1142 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgcggtccggcttactctactgttttggactattggggccagggaaccctggtcaccgtctcctca

SEQ ID NO: 105 - DNA, 321 bp, Artificial Sequence
  antibody L chain 1135 V region
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaa

SEQ ID NO: 106 - DNA, 348 bp, Artificial Sequence
  antibody H chain 1148 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgcgctgttttcggttttgactattggggccagggaaccctggtcaccgtctcctca

SEQ ID NO: 107 - DNA, 321 bp, Artificial Sequence
  antibody L chain 1149 V region
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacaggcttactacttcccgcacacttttggccaggggaccaagctggagatcaaa

SEQ ID NO: 108 - DNA, 357 bp, Artificial Sequence
  antibody H chain 1138 V region
gaggtgcagctgttggagagcgggggaggcttggtacagcctggggggtccctgcgcctctcctgtgcagccagcggattcacctttagcagctatgccatgagctgggtccgccaggctccagggaaggggctggagtgggtctcagctattagtggtagtggtggtagcacatactatgcagactccgtgaagggccggttcaccatctcccgtgacaattccaagaacacgctgtatctgcaaatgaacagcctgcgtgccgaggacacggctgtatattattgtgcgcgcggtttcgtttactcttcttacattgactattggggccagggaaccctggtcaccgtctcctca

SEQ ID NO: 109 - DNA, 321 bp, Artificial Sequence; identical to SEQ ID NO: 105
  antibody L chain 1135 V region

SEQ ID NO: 110 - protein, 336 aa, Artificial Sequence
  bispecific molecule 1107/1145
DIQMTQSPSSLSASVGDRVTITCRASQSISSYLNWYQQKPGKAPKLLIYAASSLQSGVPSRFSGSGSGTDFTLTISSLQPEDFATYYCQQYGVYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 111 - protein, 337 aa, Artificial Sequence
  bispecific molecule 1136/1143
DIQMTQSPSSLSASVGDRVTITCRASQSISSYLNWYQQKPGKAPKLLIYAASSLQSGVPSRFSGSGSGTDFTLTISSLQPEDFATYYCQQAYYAGLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 112 - protein, 336 aa, Artificial Sequence
  bispecific molecule 1132/1139
DIQMTQSPSSLSASVGDRVTITCRASQSISSYLNWYQQKPGKAPKLLIYAASSLQSGVPSRFSGSGSGTDFTLTISSLQPEDFATYYCQQYGRNPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 113 - protein, 336 aa, Artificial Sequence
  bispecific molecule 1140/1141
DIQMTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 114 - protein, 336 aa, Artificial Sequence
  bispecific molecule 1150/1152
DIQMTQSPSSLSASVGDHVTITCRASQSISSYLNWYQQKPGKAPKLLIYAASSLQSGVPSRFSGSGSGTDFTLTISSLQPEDFATYYCQQYGSAPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECSGGGGSGGGGSAPLKIQAYFNETADLPCQFANSQNLSLSELVVFWQDQENLVLNEVYLGKERFDSVDSKYMGRTSFDSDSWTLRLHNLQIKDKGRYQCIIHHKKPTGMINIHQMNSELSVLA

SEQ ID NO: 115 - DNA, 1373 bp, Artificial Sequence
  bispecific molecule 1107/1145
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtgtttacccgttcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 116 - DNA, 1376 bp, Artificial Sequence
  bispecific molecule 1136/1143
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacaggcttactacgctggtctgttcacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 117 - DNA, 1373 bp, Artificial Sequence
  bispecifici molecule 1132/1139
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggtcgtaacccgcccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 118 - DNA, 1373 bp, Artificial Sequence
  bispecific molecule 1140/1141
gacatccagatgacccagtctccatcctccctgagcgcatctgtaggagaccgcgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagagttacagtaccccttatacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 119 - DNA, 1373 bp, Artificial Sequence
  bispecific molecule 1151/1150
gacatccagatgacccagtctccatcctccctgagcgcatctgtaggagaccacgtcaccatcacttgccgggcaagtcagagcattagcagctatttaaattggtatcagcagaaaccagggaaagcccctaagctcctgatctatgctgcatccagtttgcaaagtggggtcccatcacgtttcagtggcagtggaagcgggacagatttcactctcaccatcagcagtctgcaacctgaagattttgcaacttattactgtcaacagtacggttctgctccgcccacttttggccaggggaccaagctggagatcaaacgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtagcggaggaggaggaagcggaggaggaggaagcgcccccctcaaaatccaagcgtacttcaacgaaactgcagacttaccgtgtcagtttgccaattcgcagaatctgagcctgagcgaactggtggttttctggcaggatcaggagaacctggttctgaacgaagtctatctgggcaaagagcggttcgacagcgtggacagcaagtatatgggccgcaccagctttgatagcgacagctggaccctgcgtctgcacaatctgcaaatcaaagataagggtaggtaccagtgcattatccaccataagaagccgacgggtatgattaatattcaccaaatgaactccgagttgtctgtcctggcg

SEQ ID NO: 120 - protein, 223 aa, mus musculus
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVSAVSLSKMLKKRSPLTTGVYVKMPPTEPECEKQFQPYFIPIN

SEQ ID NO: 121 - protein, 218 aa, mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQVTTMNMTPRRPGLTRKPYQPYAPARDFAAYRP

(19) United States
(12) Patent Application Publication
(10) Pub. No.: US 2017/0304437 A1
(43) Pub. Date: Oct. 26, 2017

(54) BISPECIFIC MOLECULES CAPABLE OF SPECIFICALLY BINDING TO BOTH CTLA-4 AND CD40

(71) Applicant: ALLIGATOR BIOSCIENCE AB, Lund (SE)

(72) Inventors: Peter ELLMARK, Lund (SE); Christina FUREBRING, Lund (SE); Per NORLEN, Limhamn (SE)

(21) Appl. No.: 14/901,222

(22) PCT Filed: Jun. 25, 2014
(86) PCT No.: PCT/EP2014/063443

§ 371 Date: Dec. 28, 2015

(30) Foreign Application Priority Data
Jun. 27, 2013 (GB) 1311487.1

(51) Int. Cl. A61K 39/395; C07K 16/28; A61K 39/00

(52) U.S. Cl. CPC A61K 39/3955; C07K 16/2878; C07K 16/2818; A61K 2121/00; A61K 2039/507

(57) ABSTRACT

The present disclosure relates to bispecific molecules that specifically bind to both CD40 and CTLA-4, and in particular to both human CD40 and human CTLA-4.

### FIELD OF THE INVENTION

The present invention relates to bispecific molecules that specifically bind to both CD40 and CTLA-4, and in particular to both human CD40 and human CTLA-4.

### BACKGROUND TO THE INVENTION

Cancer is a leading cause of premature deaths in the developed world. The aim of immunotherapy in cancer is to mount an effective immune response by the body against a tumour. This may be achieved by, for example, breaking tolerance against tumour antigen, augmenting anti-tumor immune responses, and stimulating local cytokine responses at the tumor site. The key effector cell of a long lasting anti-tumor immune response is the activated tumor specific effector T cell. Incomplete activation of effector T cells by, for example, dendritic cells can cause T-cell anergy, which results in an inefficient anti-tumor response, whereas adequate induction by dendritic cells can generate a potent expansion of activated effector T cells, redirecting the immune response towards the tumor.

The cell surface CD40 receptor molecule is a member of the tumour necrosis factor receptor superfamily (TNFR) and is a key regulator in both innate and adaptive immune responses. It is expressed on human antigen presenting cells, in particular B cells, dendritic cells and macrophages, as well as on normal cells, such as fibroblasts, smooth muscle cells, endothelial cells and epithelial cells. Moreover, is it expressed on a wide range of tumor cells including all B-lymphomas, 30-70% of solid tumours, melanomas and carcinomas.

The natural ligand of CD40, designated CD154 or CD40L, is mainly expressed on mature T lymphocytes. CD40L-mediated signalling triggers several biological events, including immune cell activation, proliferation, and production of cytokines and chemokines. Thus, stimulation via the CD40 receptor enhances cellular and immune functions. Its role in cell-mediated immune responses is well known. For example, the activation of dendritic cells via CD40 stimulation, induces activation of effector T cells. Treatment with CD40 agonists may thus provide the means to redirect the immune response and expand effector T cells directed to tumour

Antitumour effects have been reported for some anti-CD40 antibodies, with several mechanisms having been identified. An indirect effect is observed for CD40 negative tumors, involving the activation of antigen presenting cells, in particular increased activity by tumor specific cytotoxic T lymphocytes and natural killer cells (NK cells). A direct antitumor mechanism is observed for CD40 positive tumours, wherein the CD40 antibody binding to tumour cells induces cell apoptosis. These mechanisms for anti-tumour activity may be complemented by the stimulation of a humoral response leading to enhanced antibody mediated cellular cytotoxicity (ADCC). However, the systemic administration of anti-CD40 antibodies has alo been associated with adverse side effects, such as shock syndrome and cytokine release syndrome.

The T cell receptor CTLA-4, serves as a negative regulator of T cell activation, and is upregulated on the T-cell surface following initial activation. The ligands of the CTLA-4 receptor, which are expressed by antigen presenting cells are the B7 proteins. The corresponding ligand receptor pair which is responsible for the upregulation of T cell activation is CD28-B7. Signalling via CD28 constitutes a costimulatory pathway, and follows upon the activation of T cells, through the T cell receptor recognizing antigenic peptide presented by the MHC complex.

By blocking the CTLA-4 interaction to the B7-1 and, or B7-2 ligands, one of the normal check points of the immune response may be removed. Clinical studies have demonstrated that CTLA-4 blockade generates anti-tumor effects. However, as with CD40, administration of anti-CTLA-4 antibodies has been associated with toxic side-effects.

There exists a need for an alternative to the existing mono-specific drugs which target either CD40 or CTLA-4.

### SUMMARY OF THE INVENTION

The present inventors have produced novel bispecific binding molecules which target two immunoregulatory receptors, CTLA-4 and CD40. The CTLA-4 and CD40 are preferably human, but may be CTLA-4 and/or CD40 from another mammal such as a non-human primate or a mouse. The non-human primate may be, for example, a cynomolgus monkey.

The suppression of immune responses observed in cancer patients may be overcome by the blocking the CTLA-4 mediated inhibition of T-cells specific for tumor antigens and, simultaneously, the targeting of CD40 will potentiate dendritic cells, B lymphocytes and macrophages cells expressing CD40. This will promote further immune responses towards tumours.

The bispecific binding molecules of the present invention may provide additional therapeutic effect by physically linking the CD40 expressing antigen presenting cells and the CTLA-4 expressing cells T cells, potentially creating a cell-cell synapse not normally found in nature. This will result in a very powerful immune activation for the immediate generation of tumoricidal activity. The bispecific binding molecules of the present invention may display higher potency and/or efficacy in cancer immunotherapy than treatment regimens which include a combination of mono-specific drugs targeting either CTLA-4 or CD40.

The bispecific binding molecule of the present invention is a polypeptide. Thus, the present invention provides a polypeptide capable of specifically binding to both human CTLA-4 and human CD40, said binding molecule comprising B1 and B2, wherein:

B1 is an antibody, or antigen binding fragment thereof, specific for human CD40; and
B2 is a polypeptide binding domain specific for human CTLA-4, which comprises or consists of (i) the amino acid sequence of SEQ ID NO: 3; or (ii) an amino acid sequence in which at least one amino acid is changed when compared to the amino acid sequence of SEQ ID NO: 3 provided that said binding domain binds to human CTLA-4 with higher affinity than wild-type human CD86. The CD40 binding domain(s) of B1 and the CTLA-4-binding domain of B2 may be the only binding domains in the polypeptide of the invention.

Also provided is a polypeptide of the invention for use in a method of treating or preventing a disease or condition in an individual.

Also provided is a method of treating or preventing a disease or condition in an individual, the method comprising administering to said individual a polypeptide according to the invention and thereby treating or preventing the disease or condition.

Also provided is a polypeptide of the invention for use in the manufacture of a medicament for treating or preventing a disease or condition in an individual.

Also provided is a polynucleotide encoding a polypeptide of the invention, and a vector or cell comprising a said polynucleotide. Also provided is a method of producing a polypeptide of the invention, comprising expressing a said polynucleotide in a cell.

Also provided is a composition comprising a polypeptide of the invention and at least one pharmaceutically acceptable diluent or carrier.

### BRIEF DESCRIPTION OF THE SEQUENCE LISTING

SEQ ID NO: 1 is the amino acid sequence of human CTLA-4 (corresponding to GenBank: AAD00698.1)

SEQ ID NO: 2 is the amino acid sequence of human CD28 (corresponding to GenBank: AAA51944.1)

SEQ ID NO: 3 is the amino acid sequence of the monomeric extracellular domainof human wildtype CD86, excluding a 23 amino acid signal sequence from the N terminus.

SEQ ID NO: 4 is the amino acid sequence of the monomeric extracellular and transmembrane domains of human wildtype CD86, including N-terminal signal sequence. All numbering of amino acid positions herein is based on the positions in SEQ ID NO: 4 starting from the N terminus. Thus, the Alanine at the N terminus of SEQ ID NO: 3 is numbered 24.

SEQ ID NO: 5 is the amino acid sequence of a mutant form of the extracellular domain of human CD86 disclosed in Peach et al (Journal of Biological Chemistry 1995, vol 270(36), 21181-21187). H at position 79 of the wild type sequence is susbstituted with A in the corresponding position for the sequence of SEQ ID NO: 5. This change is referred to herein as H79A. Equivalent nomenclature is used throughout for other amino acid substitutions referred to herein. Numbering of positions is based on SEQ ID NO: 4 as outlined above.

SEQ ID NOs: 6 to 24 are the amino acid sequences of specific proteins of the invention.

SEQ ID NOs: 25 to 43 are nucleotide sequences encoding the amino acid sequences of each of SEQ ID NOs 6 to 24, respectively

SEQ ID NO: 44 is the the full length amino acid sequence of human CD86 (corresponding to GenBank: ABK41931.1)

SEQ ID NO: 45 is the amino acid sequence of human CD40 (corresponding to GenBank: AAH12419.1)

SEQ ID NOs: 46 to 49 are various linkers which may be used in the polypeptides of the invention.

SEQ ID NO: 50, 51 and 52 are the amino acid sequences of CDRs 1, 2 and 3 respectively of the heavy chain of the antibody A2-54.

SEQ ID NO: 53, 54 and 55 are the amino acid sequences of CDRs 1, 2 and 3 respectively of the light chain of the antibody A2-54.

SEQ ID NOs: 56 to 60 and 110 to 114 are the amino acid sequences of exemplary polypeptides of the invention.

SEQ ID NO: 61 is an amino acid sequence of the heavy chain of the antibody A2-54.

SEQ ID NO: 62 is an amino acid sequence of the the light chain of the antibody A2-54.

SEQ ID NO: 63 is a nucleotide sequence encoding SEQ ID NO. 61.

SEQ ID NO: 64 is a nucleotide sequence encoding SEQ ID NO. 62.

SEQ ID NOs: 65 to 69 and 115 to 119 are nucleotide sequences encoding SEQ ID NOs: 56 to 60 and 110 to 114, respectively.

SEQ ID NOs: 70, 72, 74, 76, 78, 80, 82, 84, 86 and 88 are amino acid sequences of the heavy chain of antibodies disclosed herein.

SEQ ID NOs: 71, 73, 75, 77, 79, 81, 83, 85, 87 and 89 are amino acid sequences of the light chain of antibodies disclosed herein.

SEQ ID NOs: 90, 92, 94, 96, 98, 100, 102, 104, 106 and 108 are nucleic acid sequences encoding heavy chain sequences of antibodies disclosed herein.

SEQ ID NOs: 91, 93, 95, 97, 99, 101, 103, 105, 107 and 109 are nucleic acid sequences encoding heavy chain sequences of antibodies disclosed herein.

SEQ ID NO: 120 is the amino acid sequence of murine CTLA-4 (corresponding to UniProtKB/Swiss-Prot: P09793.1).

SEQ ID NO: 121 is the amino acid sequence of murine CD28 (corresponding to GenBank: AAA37395.1).

### BRIEF DESCRIPTION OF THE FIGURES

FIG. 1 shows the CTLA-4 binding properties of CTLA-4 binding domains of polypeptides the invention as determined by an ELISA binding assay.

FIG. 2 shows the CTLA-4 binding properties of CTLA-4 binding domains of polypeptides of the invention as determined by an ELISA inhibition assay.

FIG. 3 shows a schematic representation of the structure of exemplary polypeptides of the invention: A is 956/530 and 957/530; B is 959/530 and 960/530; C is 958/531. Anti-CD40 antibody variable domains are filled in black; constant domains in white. CTLA-A binding domains are shaded with diagonal lines.

FIG. 4 provides a schematic representation of human wild-type CD86 amino acid sequences disclosed herein. A is the amino acid sequence of the monomeric soluble extracellular domain of human CD86 without N-terminal signal sequence (SEQ ID NO: 3); B is the amino acid sequence of the monomeric extracellular and transmembrane domains of human wildtype CD86, including N-terminal signal sequence (SEQ ID NO: 4); C is the full length amino acid sequence of human CD86 (Genbank ABK41931.1; SEQ ID NO: 44). The sequence in A may optionally lack Alanine and Proline at the N terminus, that is positions 24 and 25, shown in bold. Signal sequences in B and C are underlined. Numbering of amino acid positions is based on SEQ ID NOs: 4 and 44, starting from the N terminus.

FIG. 5 shows the CTLA-4 binding properties of exemplary polypeptides of the invention as determined by an ELISA binding assay.

FIG. 6 shows the CD40 binding properties of exemplary polypeptides of the invention as determined by an ELISA binding assay.

FIG. 7 shows the ability of exemplary polypeptides of the invention to simultaneously bind to both CTLA-4 and CD40 in an ELISA assay.

FIG. 8 shows the ability of exemplary polypeptides of the invention to simultaneously bind to both CTLA-4 and CD40 as measured using Surface Plasmon Resonance (SPR).

FIG. 9 is a representative FACS plot showing the interaction between CD40-expressing and CTLA-4 expressing cells as a result of binding by an exemplary polypeptide of the invention.

FIG. 10 shows the ability of an exemplary polypeptide of the invention to induce immune activation, as indicated by B cell proliferation. A synergistic effect is observed for the polypeptide of the invention 957/530.

FIG. 11 shows the results of an inhibition ELISA demonstrating that a polypeptide of the invention has binding affinity of a similar magnitude for both human and murine CTLA-4.

FIG. 12A-C show the CD40 binding properties of exemplary polypeptides of the invention as determined by an ELISA binding assay.

FIG. 13A-F show the CTLA-4 binding properties of exemplary polypeptides of the invention as determined by an ELISA binding assay.

FIG. 14 shows the the ability of exemplary polypeptides of the invention to simultaneously bind to both CTLA-4 and CD40 as measured using Surface Plasmon Resonance (SPR).

FIG. 15 shows the in vivo anti-tumour effect of an exemplary polypeptide of the invention. Increased survival (top panel) and decreased tumour volume (bottom panel) relative to controls is demonstrated.

FIG. 16 shows that treatment with an exemplary polypeptide of the invention established immunological memory in vivo. Previously treated mice have increased survival (top panel) and decreased tumour volume (bottom panel) relative to controls.

FIG. 17 shows that exemplary polypeptides of the invention have increased efficacy at inducing B cell proliferation, when compared to the corresponding monospecific antibodies. The y axis shows the mean fold change in B cell proliferation for each polypeptide of the invention relative to its corresponding monospecific antibody.

FIG. 18 shows that exemplary polypeptides of the invention have increased efficacy at inducing activation of immune cells in human PBMCs, when compared to the corresponding monospecific antibodies. The y axis shows the mean fold change in IL2 production for each polypeptide of the invention relative to its corresponding monospecific antibody.

FIG. 19 shows that an exemplary polypeptide of the invention triggers B cell activation via CD40 that is enhanced by simultaneous cross linking with CTLA-4 on another cell population (in trans), as indicated by increased CD83 expression (y axis).

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “an inhibitor” includes two or more such inhibitors, or reference to “an oligonucleotide” includes two or more such oligonucleotides and the like.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

All publications, patents and patent applications cited herein, whether supra or infra, are hereby incorporated by reference in their entirety.

### The Polypeptide of the Invention Part B1—Antibody Specific for CD40

B1 is an antibody, or antigen binding fragment thereof, specific for human CD40. The term “antibody” as referred to herein includes whole antibodies and any antigen binding fragment (i.e., “antigen-binding portion”) or single chains thereof. An antibody refers to a glycoprotein comprising at least two heavy (H) chains and two light (L) chains inter-connected by disulfide bonds, or an antigen binding portion thereof Each heavy chain is comprised of a heavy chain variable region (abbreviated herein as VH) and a heavy chain constant region. Each light chain is comprised of a light chain variable region (abbreviated herein as VL) and a light chain constant region. The variable regions of the heavy and light chains contain a binding domain that interacts with an antigen. The VH and VL regions can be further subdivided into regions of hypervariability, termed complementarity determining regions (CDR), interspersed with regions that are more conserved, termed framework regions (FR). The constant regions of the antibodies may mediate the binding of the immunoglobulin to host tissues or factors, including various cells of the immune system (e.g., effector cells) and the first component (Clq) of the classical complement system.

The term “antigen-binding fragment” of an antibody refers to one or more fragments of an antibody that retain the ability to specifically bind to an antigen, such as CD40. It has been shown that the antigen-binding function of an antibody can be performed by fragments of a full-length antibody. Examples of binding fragments encompassed within the term “antigen-binding portion” of an antibody include a Fab fragment, a F(ab′)₂ fragment, a Fab′ fragment, a Fd fragment, a Fv fragment, a dAb fragment and an isolated complementarity determining region (CDR). Single chain antibodies such as scFv and heavy chain antibodies such as VHH and camel antibodies are also intended to be encompassed within the term “antigen-binding portion” of an antibody. These antibody fragments may be obtained using conventional techniques known to those of skill in the art, and the fragments may be screened for utility in the same manner as intact antibodies.

The polypeptide of the invention may incorporate any anti-CD40 antibody, or antigen binding fragment therof. Such an anti-CD40 antibody may be produced by an suitable means. For example, such an antibody may be prepared, expressed, created or isolated by recombinant means, such as (a) antibodies isolated from an animal (e.g., a mouse) that is transgenic or transchromosomal for the immunoglobulin genes of interest or a hybridoma prepared therefrom, (b) antibodies isolated from a host cell transformed to express the antibody of interest, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences. Alternatively an anti-CD40 antibody may be produced by methods comprising immunising a non-human animal with CD40, or immunising human lymphocytes in vitro with CD40.

The antibody typically comprises at least one heavy chain variable domain (V_{H}) and at least one light chain variable domain (V_{L}). The antibody may comprise an Fc region, preferably a human Fc region, or a variant of a said region. The Fc region may be an IgGl, IgG2, IgG3 or IgG4 region, preferably an IgG1 or IgG4 region. A variant of an Fc region typically binds to Fc receptors, sucha as FcgammaR and/or neonatal Fc receptor (FcRn) with altered affinity providing for improved function and/or half life of the polypeptide. The half life may be either increased or a decreased relative to the half life of a polypeptide comprising a native Fc region.

Heavy and light chain amino acid sequences of preferred antibodies for incorporation in the polypeptide of the invention are shown in table A. Within each sequence the CDR sequences are underlined. The sequences are all presented in the orientation N to C from left to right. Thus, reading from left to right within each sequence, CDR1 is the first underlined, CDR2 is the second underlined, and CDR3 is the third underlined sequence. Constant domains are shown in italics.

TABLE A

| | | |
|---|---|---|
| SEQ | | |
| ID | Chain | Sequence |

Antibody A2-54

| | | |
|---|---|---|
| 61 | A2-54 | EVQLLESGGGLVQPGGSLRLSCAASGFTFSDYYMSWVRQAPGKGLEWISSISSNG |
| | Variable | IYIYYADSLKGRFTISRDNSKNTLYLQMNSLRAEDTAVYYCARAPVDYSNPSGMD |
| | Heavy, | VWGQGTLVTVSSASTKGPSVFPLAPSSKSTSGGTAALGCLVKDYFPEPVTVSWNS |
| | VH- | GALTSGVHTFPAVLQSSGLYSLSSVVTVPSSSLGTQTYICNVNHKPSNTKVDKKV |
| | gamma1 | EPKSCDKTHTCPPCPAPELLGGPSVELFPPKPKDTLMISRTPEVTCVVVDVSEED |
| | | PEVKFNWYVDGVEVHNAKTKPREEQYNSTYRVVSVLTVLHQDWLNGKEYKCKVSN |
| | | KALPAPIEKTISKAKGQPREPQVYTLPPSRDELTKNOVSLTCLVKGFYPSDIAVE |
| | | WESNGQPENNYKTTPPVLDSDGSFFLYSKLTVDKSRWQQGNVFSCSVMHEALHNH |
| | | YTQKSLSLSPGK |
| 62 | A2-54 | QSVLTQPPSASGTPGQRVTISCSGSSSNIGSNTVNWYQQLPGTAPKLLIYRNNQR |
| | Variable | PSGVPDRFSGSKSGTSASLAISGLRSEDEADYYCAAWDDSLSGWRFGGGTKLTVL |
| | Light, | GGTVAAPSVFIFPPSDEOLKSGTASVVCLLNNFYPREAKVQWKVDNALOSGNSQE |
| | VL-kappa | SVTEQDSKDSTYSLSSTLTLSKADYEKHKVYACEVTHQGLSSPVTKSFNRGEC |

Antibody 1107/1145

| | | |
|---|---|---|
| 70 | Antibody | EVQLLESGGGLVQPGGSLRLSCAASGFTFSSYAMSWVRQAPGKGLEWVSAISGSG |
| | clone, | GSTYYADSVKGRFTISRDNSKNTLYLQMNSLRAEDTAVYYCARRVWGFDYWGQGT |
| | 1107, | LVTVSSASTKGPSVFPLAPSSKSTSGGTAALGCLVKDYFPEPVTVSWNSGALTSG |
| | variable | VHTFPAVLQSSGLYSLSSVVTVPSSSLGTQTYICNVNHKPSNTKVDKKVEPKSCD |
| | heavy, | KTHTCPPCPAPELLGGPSVFLFPPKPKDTLMISRTPEVTCVVVDVSREDPEVKFN |
| | VH- | WYVDGVEVHNAKTKPREEQYNSTYRVVSVLTVLHQDWLNGKEYKCKVSNKALPAP |
| | gamma1 | IEKTISKAKGQPREPQVYTLPPSRDELTKNOVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |
| 71 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQYGVYPFTFGQGTKLEIKRTV |
| | 1145, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1136/1143

| | | |
|---|---|---|
| 72 | Antibody | EVQLLESGGGLVQPGGSLRLSCAASGFTFSSYAMSWVRQAPGKGLEWVSAISGSG |
| | clone, | GSTYYADSVKGRFTISRDNSKNTLYLQMNSLRAEDTAVYYCARYVFGIDYWGQGT |
| | 1136, | LVTVSSASTKGPSVFPLAPSSKSTSGGTAALGCLVKDYFPEPVTVSWNSGALTSG |
| | variable | VHTFPAVLQSSGLYSLSSVVTVPSSSLGTQTYICNVNHKPSNTKVDKKVEPKSCD |
| | heavy, | KTHTCPPCPAPELLGGPSVFLFPPKPKDTLMISRTPEVTCVVVDVSREDPEVKFN |
| | VH- | WYVDGVEVHNAKTKPREEQYNSTYRVVSVLTVLHQDWLNGKEYKCKVSNKALPAP |
| | gamma1 | IEKTISKAKGQPREPQVYTLPPSRDELTKNOVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |
| 73 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQAYYAGLFTFGQGTKLEIKRT |
| | 1143, | VAAPSVFIFPPSDEOLKSGTASVVCLLNNFYPREAKVQWKVDNALOSGNSQESVT |
| | variable | EQDSKDSTYSLSSTLTLSKADYEKHKVYACEVTHQGLSSPVTKSFNRGEC |
| | light, | |
| | VL-kappa | |

Antibody 1132/1139

| | | |
|---|---|---|
| 74 | Antibody | EVQLLESGGGLVQPGGSLRLSCAASGFTFSSYAMSWVRQAPGKGLEWVSGIGSYG |
| | clone | GGTYYADSVKGRFTISRDNSKNTLYLQMNSLRAEDTAVYYCARYVNFGMDYWGQG |
| | 1132, | TLVTVSSASTKGPSVFPLAPSSKSTSGGTAALGCLVKDYFPEPVTVSWNSGALTS |
| | variable | GVHTFPAVLQSSGLYSLSSVVTVPSSSLGTQTYICNVNHKPSNTKVDKKVEPKSC |
| | heavy, | DKTHTCPPCPAPELLGGPSVFLFPPKPKDTLMISRTPEVTCVVVDVSREDPEVKF |
| | VH- | NWYVDGVEVHNAKTKPREEQYNSTYRVVSVLTVLHQDWLNGKEYKCKVSNKALPA |
| | gamma1 | PIEKTISKAKGQPREPQVYTLPPSRDELTKNOVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |
| 75 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQYGRNPPTFGQGTKLEIKRTV |
| | 1139, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1140/1141

| | | |
|---|---|---|
| 76 | Antibody | EVQLLESGGGLVQPGGSLRLSCAASGFTFSSYAMSWVRQAPGKGLEWVSAISGSG |
| | clone | GSTYYADSVKGRFTISRDNSKNTLYLQMNSLRAEDTAVYYCARGPVYSSVFDYWG |
| | 1140, | QGTLVTVSSASTKGPSVFPLAPSSKSTSGGTAALGCLVKDYFPEPVTVSWNSGAL |
| | variable | TSGVHTFPAVLQSSGLYSLSSVVTVPSSSLGTQTYICNVNHKPSNTKVDKKVEPK |
| | heavy, | SCDKTHTCPPCPAPELLGGPSVFLFPPKPKDTLMISRTPEVTCVVVDVSREDPEV |
| | VH- | KFNWYVDGVEVHNAKTKPREEQYNSTYRVVSVLTVLHQDWLNGKEYKCKVSNKAL |
| | gamma1 | PAPIEKTISKAKGQPREPQVYTLPPSRDELTKNOVSLTCLVKGFYPSDIAVEWES |
| | | NGQPENNYKTTPPVLDSDGSFFLYSKLTVDKSRWQQGNVFSCSVMHEALHNHYTQ |
| | | KSLSLSPGK |
| 77 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQSYSTPYTFGQGTKLEIKRTV |
| | 1141, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1150/1152

| | | |
|---|---|---|
| 78 | Antibody | EVQLLESGGGLVQPGGSLRLSCAASGFTFSSYAMSWVRQAPGKGLEWVSGIGGSS |
| | clone | SYTSYADSVKGRFTISRDNSKNTLYLQMNSLRAEDTAVYYCARYYSYHMDYWGQG |
| | 1150, | TLVTVSSASTKGPSVFPLAPSSKSTSGGTAALGCLVKDYFPEPVTVSWNSGALTS |
| | variable | GVHTFPAVLQSSGLYSLSSVVTVPSSSLGTQTYICNVNHKPSNTKVDKKVEPKSC |
| | heavy, | DKTHTCPPCPAPELLGGPSVFLFPPKPKDTLMISRTPEVTCVVVDVSREDPEVKF |
| | VH- | NWYVDGVEVHNAKTKPREEQYNSTYRVVSVLTVLHQDWLNGKEYKCKVSNKALPA |
| | gamma1 | PIEKTISKAKGQPREPQVYTLPPSRDELTKNOVSLTCLVKGFYPSDIAVEWESNG |
| | | QPENNYKTTPPVLDSDGSFFLYSKLTVDKSRWQQGNVFSCSVMHEALHNHYTQKS |
| | | LSLSPGK |
| 79 | Antibody | DIQMTQSPSSLSASVGDHVTITCRASQSISSYLNWYQQKPGKAPKLLIYAASSLQ |
| | clone | SGVPSRFSGSGSGTDFTLTISSLQPEDFATYYCQQYGSAPPTFGQGTKLEIKRTV |
| | 1152, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1134/1141

| | | |
|---|---|---|
| 80 | Antibody | EVQLLESGGGLVQPGGSLRLSCAASGFTFSSYAMSWVRQAPGKGLEWVSSIYSGG |
| | clone | GGTSYADSVKGRFTISRDNSKNTLYLQMNSLRAEDTAVYYCARGPAYSSFFDYWG |
| | 1134, | QGTLVTVSSASTKGPSVFPLAPSSKSTSGGTAALGCLVKDYFPEPVTVSWNSGAL |
| | variable | TSGVHTFPAVLQSSGLYSLSSVVTVPSSSLGTQTYICNVNHKPSNTKVDKKVEPK |
| | heavy, | SCDKTHTCPPCPAPELLGGPSVFLFPPKPKDTLMISRTPEVTCVVVDVSREDPEV |
| | VH- | KFNWYVDGVEVHNAKTKPREEQYNSTYRVVSVLTVLHQDWLNGKEYKCKVSNKAL |
| | gamma1 | PAPIEKTISKAKGQPREPQVYTLPPSRDELTKNOVSLTCLVKGFYPSDIAVEWES |
| | | NGQPENNYKTTPPVLDSDGSFFLYSKLTVDKSRWQQGNVFSCSVMHEALHNHYTQ |
| | | KSLSLSPGK |
| 81 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQSYSTPYTFGQGTKLEIKRTV |
| | 1141, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1146/1147

| | | |
|---|---|---|
| 82 | Antibody | EVQLLESGGGLVQPGGSLRLSCAASGFTFSSYAMSWVRQAPGKGLEWVSAISGSG |
| | clone | GSTYYADSVKGRFTISRDNSKNTLYLQMNSLRAEDTAVYYCARRVFGFDYWGQGT |
| | 1146, | LVTVSSASTKGPSVFPLAPSSKSTSGGTAALGCLVKDYFPEPVTVSWNSGALTSG |
| | variable | VHTFPAVLQSSGLYSLSSVVTVPSSSLGTQTYICNVNHKPSNTKVDKKVEPKSCD |
| | heavy, | KTHTCPPCPAPELLGGPSVFLFPPKPKDTLMISRTPEVTCVVVDVSREDPEVKFN |
| | VH- | WYVDGVEVHNAKTKPREEQYNSTYRVVSVLTVLHQDWLNGKEYKCKVSNKALPAP |
| | gamma1 | IEKTISKAKGQPREPQVYTLPPSRDELTKNOVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |
| 83 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQYYYYPFTFGQGTKLEIKRTV |
| | 1147, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1142/1135

| | | |
|---|---|---|
| 84 | Antibody | EVQLLESGGGLVQPGGSLRLSCAASGFTFSSYAMSWVRQAPGKGLEWVSAISGSG |
| | clone | GSTYYADSVKGRFTISRDNSKNTLYLQMNSLRAEDTAVYYCARGPAYSTVLDYWG |
| | 1142, | QGTLVTVSSASTKGPSVFPLAPSSKSTSGGTAALGCLVKDYFPEPVTVSWNSGAL |
| | variable | TSGVHTFPAVLQSSGLYSLSSVVTVPSSSLGTQTYICNVNHKPSNTKVDKKVEPK |
| | heavy, | SCDKTHTCPPCPAPELLGGPSVFLFPPKPKDTLMISRTPEVTCVVVDVSHEDPEV |
| | VH- | KFNWYVDGVEVHNAKTKPREEQYNSTYRVVSVLTVLHQDWLNGKEYKCKVSNKAL |
| | gamma1 | PAPIEKTISKAKGQPREPQVYTLPPSRDELTKNOVSLTCLVKGFYPSDIAVEWES |
| | | NGQPENNYKTTPPVLDSDGSFFLYSKLTVDKSRWQQGNVFSCSVMHEALHNHYTQ |
| | | KSLSLSPGK |
| 85 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQSYSTPYTFGQGTKLEIKRTV |
| | 1135, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1148/1149

| | | |
|---|---|---|
| 86 | Antibody | EVQLLESGGGLVQPGGSLRLSCAASGFTFSSYAMSWVRQAPGKGLEWVSAISGSG |
| | clone | GSTYYADSVKGRFTISRDNSKNTLYLQMNSLRAEDTAVYYCARAVFGFDYWGQGT |
| | 1148, | LVTVSSASTKGPSVFPLAPSSKSTSGGTAALGCLVKDYFPEPVTVSWNSGALTSG |
| | variable | VHTFPAVLQSSGLYSLSSVVTVPSSSLGTQTYICNVNHKPSNTKVDKKVEPKSCD |
| | heavy, | KTHTCPPCPAPELLGGPSVFLFPPKPKDTLMISRTPEVTCVVVDVSHEDPEVKFN |
| | VH- | WYVDGVEVHNAKTKPREEQYNSTYRVVSVLTVLHQDWLNGKEYKCKVSNKALPAP |
| | gamma1 | IEKTISKAKGQPREPQVYTLPPSRDELTKNOVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |
| 87 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQAYYFPHTFGQGTKLEIKRTV |
| | 1149, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

Antibody 1138/1135

| | | |
|---|---|---|
| 88 | Antibody | EVQLLESGGGLVQPGGSLRLSCAASGFTFSSYAMSWVRQAPGKGLEWVSAISGSG |
| | clone | GSTYYADSVKGRFTISRDNSKNTLYLQMNSLRAEDTAVYYCARGFVYSSYIDYWG |
| | 1138, | QGTLVTVSSASTKGPSVFPLAPSSKSTSGGTAALGCLVKDYFPEPVTVSWNSGAL |
| | variable | TSGVHTFPAVLQSSGLYSLSSVVTVPSSSLGTQTYICNVNHKPSNTKVDKKVEPK |
| | heavy, | SCDKTHTCPPCPAPELLGGPSVFLFPPKPKDTLMISRTPEVTCVVVDVSHEDPEV |
| | VH- | KFNWYVDGVEVHNAKTKPREEQYNSTYRVVSVLTVLHQDWLNGKEYKCKVSNKAL |
| | gamma1 | PAPIEKTISKAKGQPREPQVYTLPPSRDELTKNOVSLTCLVKGFYPSDIAVEWES |
| | | NGQPENNYKTTPPVLDSDGSFFLYSKLTVDKSRWQQGNVFSCSVMHEALHNHYTQ |
| | | KSLSLSPGK |
| 89 | Antibody | DIQMTQSPSSLSASVGDRVTITCRASQSISSYLNWYQQKPGKAPKLLIYAASSLQ |
| | clone | SGVPSRFSGSGSGTDFTLTISSLQPEDFATYYCQQSYSTPYTFGQGTKLEIKRTV |
| | 1135, | AAPSVFIFPPSDEOLKSGTASVVCLLNNFYPREAKVQWKVDNALOSGNSQESVTE |
| | variable | QDSKDSTYSLSSTLTLSKADYEKHKVYACEVTHOGLSSPVTKSPNRGEC |
| | light, | |
| | VL-kappa | |

The CDRs for the heavy chain of antibody A2-54 are also shown in SEQ ID NOs: 50, 51 and 52. The CDRs for the light chain are also shown in SEQ ID NOs: 53, 54 and 55. All six CDRs of A2-54 are also shown in the following table.

| SEQ | | |
|---|---|---|
| ID | CDR | Sequence |
| 50 | VH1 | GFTFSDYY |
| 51 | VH2 | ISSNGIYI |
| 52 | VH3 | ARAPVDYSNPSGMDV |
| 53 | VL1 | SSNIGSNT |
| 54 | VL2 | RNN |
| 55 | VL3 | AAWDDSLSG |

The antibody may comprise the amino acid sequence of any one of the heavy chains for which the sequences are shown in Table A, or a fragment or variant of any thereof. The antibody may comprise the amino acid sequence of any one of the light chains for which the sequences are shown in Table A, or a fragment or variant of any thereof A preferred fragment of a said heavy chain or a said light chain is the variable region.

The antibody may comprise both the heavy chain (or a fragment or variant thereof) and the light chain (or a fragment or variant thereof) of any one of the antibodies shown in Table A.

The antibody may comprise a fragment of one of the heavy or light chain amino acid sequences shown in Table A. For example, an antibody of the invention may comprise a fragment of at least 7, at least 8, at least 9, at least 10, at least 12, at least 15, at least 18, at least 20 or at least 25 consecutive amino acids from said amino acid sequence. Such a fragment will preferably retain one or more of the functions discussed below, such as the ability to bind to CD40.

The antibody may comprise one, two, three, four, five or six CDR sequences from any one of the light chain or heavy chain sequences shown in Table A. The antibody may comprise one or more heavy chain CDR sequences and alternatively or additionally one or more light chain CDR sequences selected from the CDR sequences shown in Table A. The antibody may comprise one, two or all three of the heavy chain CDR sequences of any one of the antibodies shown in Table A, and alternatively or additionally one, two or all three of the light chain CDR sequences of the same said antibody as shown in Table

A. An antibody of the invention preferably comprises all six CDR sequences of an antibody as shown in Table A. For example an antibody of the invention may comprise all six CDR sequences of antibody 1107/1145 as shown in Table A.

The antibody may alternatively be or may comprise a variant of one of the specific sequences recited above. For example, a variant may be a substitution, deletion or addition variant of any of the above amino acid sequences.

A variant antibody may comprise 1, 2, 3, 4, 5, up to 10, up to 20, up to 30 or more amino acid substitutions and/or deletions from the specific sequences and fragments discussed above. “Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

Preferred “derivatives” or “variants” include those in which instead of the naturally occurring amino acid the amino acid which appears in the sequence is a structural analog thereof. Amino acids used in the sequences may also be derivatized or modified, e.g. labelled, providing the function of the antibody is not significantly adversely affected. Derivatives and variants as described above may be prepared during synthesis of the antibody or by post- production modification, or when the antibody is in recombinant form using the known techniques of site- directed mutagenesis, random mutagenesis, or enzymatic cleavage and/or ligation of nucleic acids.

Preferably variant antibodies have an amino acid sequence which has more than 60%, or more than 70%, e.g. 75 or 80%, preferably more than 85%, e.g. more than 90 or 95% amino acid identity to the V_{L} or V_{H} domain, or a fragment thereof, of an antibody disclosed herein. This level of amino acid identity may be seen across the full length of the relevant SEQ ID NO sequence or over a part of the sequence, such as across 20, 30, 50, 75, 100, 150, 200 or more amino acids, depending on the size of the full length polypeptide.

In connection with amino acid sequences, “sequence identity” refers to sequences which have the stated value when assessed using ClustalW (Thompson et al., 1994, supra) with the following parameters:

Pairwise alignment parameters -Method: accurate, Matrix: PAM, Gap open penalty: 10.00, Gap extension penalty: 0.10;

Multiple alignment parameters—Matrix: PAM, Gap open penalty: 10.00, % identity for delay: 30, Penalize end gaps: on, Gap separation distance: 0, Negative matrix: no, Gap extension penalty: 0.20, Residue-specific gap penalties: on, Hydrophilic gap penalties: on, Hydrophilic residues: GPSNDQEKR. Sequence identity at a particular residue is intended to include identical residues which have simply been derivatized.

The present invention thus provides antibodies having specific heavy and light chain amino acid sequences and variants and fragments thereof which maintain the function or activity of these chains.

Accordingly, the antibody may comprise:

(a) a heavy chain amino acid sequence of any one of SEQ ID NOs: 61, 70, 72, 74, 76, 78, 80, 82, 84, 86 or 88;

(b) a fragment of at least 7 amino acids of (a), such as the variable region, wherein the antibody retains the ability to specifically bind to CD40; or

(c) a variant of (a) having at least 70% amino acid sequence identity to a sequence of (a), wherein the antibody retains the ability to specifically bind to CD40.

The antibody may comprise:

(a) a light chain amino acid sequence of any one of SEQ ID NOs: 62, 71, 73, 75, 77, 79, 81, 83, 85, 87 or 89;

(b) a fragment of at least 7 amino acids of (a), such as the variable region, wherein the antibody retains the ability to specifically bind to CD40; or

(c) a variant of (a) having at least 70% amino acid sequence identity to a sequence of (a), wherein the antibody retains the ability to specifically bind to CD40.

The antibody may bind to the same epitope as any of the specific antibodies, fragments and variants described herein. Preferably it binds to the same epitope as an antibody as shown in Table A, or an antibody possessing all six CDRs of an antibody as shown in Table A.

The antibody, or antigen binding fragment therof, has certain preferred binding characteristics and functional effects, which are explained in more detail below. Said antibody, or antigen binding fragment thereof, preferably retrains these binding characteristics and functional effects when incorporated as part of a polypeptide of the invention.

The antibody preferably specifically binds to CD40, that is it binds to CD40 but does not bind, or binds at a lower affinity, to other molecules. The term CD40 as used herein typically refers to human CD40. The sequence of human CD40 is set out in SEQ ID NO: 45. The antibody may have some binding affinity for CD40 from other mammals, such as CD40 from a non-human primate (for example cynomolgus monkey) or a mouse. The antibody preferably binds to human CD40 when localised on the surface of a cell.

The antibody has the ability to bind to CD40 in its native state and in particular to CD40 localised on the surface of a cell. Preferably, the antibody will bind specifically to CD40. That is, an antibody of the invention will preferably bind to CD40 with greater binding affinity than that at which it binds to another molecule.

By “localised on the surface of a cell” it is meant that CD40 is associated with the cell such that one or more region of CD40 is present on the outer face of the cell surface.

For example, CD40 may be inserted into the cell plasma membrane (i.e. orientated as a transmembrane protein) with one or more regions presented on the extracellular surface. This may occur in the course of expression of CD40 by the cell. Thus, in one embodiment, “localised on the surface of a cell” may mean “expressed on the surface of a cell.” Alternatively, CD40 may be outside the cell with covalent and/or ionic interactions localising it to a specific region or regions of the cell surface.

The antibody may modulate the activity of a cell expressing CD40, wherein said modulation is an increase or decrease in the activity of said cell. The cell is typically a dendritic cell or a B cell.

Professional APCs, such as dendritic cells, are activated when signaling via CD40 occurs, which triggers several biological events, including immune cell activation, proliferation, and production of cytokines and chemokines. Methods for determining dendritic cell activation associated with CD40 are known in the art (discussed, for example, in Schonbeck et al., 2001, Cell Mol Life Sci., 58:40-43; van Kooten et al., 2000, J. Leuk., Biol., 67: 2-17) and are described further below.

Stimulation of human B cells with recombinant CD40L or anti-CD40 antibodies induces up-regulation of surface markers, such as CD23, CD30, CD80, CD86, Fas and MHC II, secretion of soluble cytokines, e.g. IL-6, TNF-γ and TNF-α, and homeotypic aggregation. Methods for determining CD40-related B cell activation are known in the art and are described further below.

Methods and assays for determining the ability of an antibody to modulate the activity of dendritic cells and B cells are well known in the art. For example, the activation of dendritic cells may be assessed by measuring the level of cell surface markers such as CD86 and CD80 and/or by measuring anti-CD40 antibody-induced secretion of IFNγ from T cells, wherein in an increase in any of these parameters indicates increased activation and a decrease represents decreased activation. Similarly, the ability of an antibody to modulate the activity of B cells may be assessed by measuring the level of cell surface markers (such as CD86) and/or by measuring anti-CD40 antibody-induced B cell proliferation , wherein in an increase in any of these parameters indicates increased activation and a decrease represents decreased activation.

The terms “binding activity” and “binding affinity” are intended to refer to the tendency of an antibody molecule to bind or not to bind to a target. Binding affinity may be quantified by determining the dissociation constant (Kd) for an antibody and its target. Similarly, the specificity of binding of an antibody to its target may be defined in terms of the comparative dissociation constants (Kd) of the antibody for its target as compared to the dissociation constant with respect to the antibody and another, non-target molecule.

Typically, the Kd for the antibody with respect to the target will be 2-fold, preferably 5-fold, more preferably 10-fold less than Kd with respect to the other, non-target molecule such as unrelated material or accompanying material in the environment. More preferably, the Kd will be 50-fold less, even more preferably 100-fold less, and yet more preferably 200-fold less.

The value of this dissociation constant can be determined directly by well-known methods, and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

A competitive binding assay can be conducted in which the binding of the antibody to the target is compared to the binding of the target by another, known ligand of that target, such as another antibody. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

An antibody of the invention is preferably capable of binding to its target with an affinity that is at least two-fold, 10-fold, 50-fold, 100-fold or greater than its affinity for binding to another non-target molecule.

The antibody may be a human antibody. The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

### The Polypeptide of the Invention Part B2—Binding Domain Specific for CTLA-4

CD86 and CD80 may be referred to herein as B7 proteins (B7-2 and B7-1 respectively). These proteins are expressed on the surface of antigen presenting cells and interact with the T cell receptors CD28 and CTLA-4. The binding of the B7 molecules to CD28 promotes T cell activation while binding of B7 molecules to CTLA-4 switches off the activation of the T cell. The interaction between the B7 proteins with CD28 and/or CTLA-4 constitute a costimulatory signalling pathway which plays an important role in immune activation and regulation. Thus, the B7 molecules are part of a pathway, amenable to manipulation in order to uncouple immune inhibition, thereby enhancing immunity in patients.

The CD86 protein is a monomer and consists of two extracellular immunoglobulin superfamily domains. The receptor binding domain of CD86 has a typical IgV-set structure, whereas the membrane proximal domain has a C1-set like structure. The structure of CD80 and CD86 have been determined on their own or in complex with CTLA-4. The contact residues on the CD80 and CD86 molecules are in the soluble extracellular domain, and mostly located in the beta-sheets and not in the (CDR-like) loops.

SEQ ID NO: 3 is the amino acid sequence of the monomeric soluble extracellular domain of human wild-type CD86. This wild type sequence may optionally lack Alanine and Proline at the N terminus, that is positions 24 and 25. These amino acids may be referred to herein as A24 and P25 respectively.

Part B2 of the polypeptide of the invention is a polypeptide binding domain specific for CTLA-4. Said binding domain may also bind to CD28. The term CTLA-4 as used herein typically refers to human CTLA-4 and the term CD28 as used herein typically refers to human CD28. The sequences of human CTLA-4 and human CD28 are set out in SEQ ID NOs: 1 and 2 respectively. Part B2 of the polypeptide of the present invention may have some binding affinity for CTLA-4 or CD28 from other mammals, for example primate or murine CTLA-4 or CD28.

Part B2 of the polypeptide of the invention has the ability to bind to CTLA-4 in its native state and in particular to CTLA-4 localised on the surface of a cell. By “localised on the surface of a cell” it is meant that CTLA-4 is associated with the cell such that one or more region of CTLA-4 is present on the outer face of the cell surface. For example, CTLA-4 may be inserted into the cell plasma membrane (i.e. orientated as a transmembrane protein) with one or more regions presented on the extracellular surface. This may occur in the course of expression of CTLA-4 by the cell. Thus, in one embodiment, “localised on the surface of a cell” may mean “expressed on the surface of a cell.” Alternatively, CTLA-4 may be outside the cell with covalent and/or ionic interactions localising it to a specific region or regions of the cell surface.

Part B2 of the polypeptide of the invention may comprise or consist of:

(i) the amino acid sequence of SEQ ID NO: 3; or

(ii) an amino acid sequence in which at least one amino acid is changed when compared to the amino acid sequence of SEQ ID NO: 3 provided that said binding domain binds to human CTLA-4 with higher affinity than wild-type human CD86.

In other words, B2 is a polypeptide binding domain specific for human CTLA-4 which comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

Accordingly, part B2 of the polypeptide of the invention may have the same target binding properties as human wild-type CD86, or may have different target binding properties compared to the target binding properties of human wild-type CD86. For the purposes of comparing such properties, “human wild-type CD86” typically refers to the monomeric soluble extracellular domain of human wild-type CD86 as described in the preceding section.

Human wild-type CD86 specifically binds to two targets, CTLA-4 and CD28. Accordingly, the binding properties of part B2 of the polypeptide of the invention may be expressed as an individual measure of the ability of the polypeptide to bind to each of these targets. For example, a polypeptide variant of the monomeric extracellular domain of human wild-type CD86 preferably binds to CTLA-4 with a higher binding affinity than that of wild-type human CD86 for CTLA-4. Such a polypeptide may optionally also bind to CD28 with a lower binding affinity than that of wild-type human CD86 for CD28.

Standard assays to evaluate the binding ability of ligands towards targets are well known in the art, including for example, ELISAs, Western blots, RIAs, and flow cytometry analysis. The binding kinetics (e.g., binding affinity) of the polypeptide also can be assessed by standard assays known in the art, such as by Surface Plasmon Resonance analysis (SPR).

The terms “binding activity” and “binding affinity” are intended to refer to the tendency of a polypeptide molecule to bind or not to bind to a target. Binding affinity may be quantified by determining the dissociation constant (Kd) for a polypeptide and its target. A lower Kd is indicative of a higher affinity for a target. Similarly, the specificity of binding of a polypeptide to its target may be defined in terms of the comparative dissociation constants (Kd) of the polypeptide for its target as compared to the dissociation constant with respect to the polypeptide and another, non-target molecule.

The value of the dissociation constant Kd can be determined directly by well-known methods, and can be computed even for complex mixtures by methods such as those, for example, set forth in Caceci et al. (Byte 9:340-362, 1984). For example, the Kd may be established using a double-filter nitrocellulose filter binding assay such as that disclosed by Wong & Lohman (Proc. Natl. Acad. Sci. USA 90, 5428-5432, 1993). A competitive binding assay can be conducted in which the binding of the polypeptide to the target is compared to the binding of the target by another, known ligand of that target, such as another polypeptide. In this case, the soluble extracellular domain of wild-type human CD86 (optionally linked to a detectable domain such as an Fc domain or an Ig domain at the N or C terminus) is a suitable alternative ligand. The concentration at which 50% inhibition occurs is known as the Ki. Under ideal conditions, the Ki is equivalent to Kd. The Ki value will never be less than the Kd, so measurement of Ki can conveniently be substituted to provide an upper limit for Kd.

Alternative measures of binding affinity include EC50 or IC50. In this context EC50 indicates the concentration at which a polypeptide achieves 50% of its maximum binding to a fixed quantity of target. IC50 indicates the concentration at which a polypeptide inhibits 50% of the maximum binding of a fixed quantity of competitor to a fixed quantity of target. In both cases, a lower level of EC50 or IC50 indicates a higher affinity for a target. The EC50 and IC50 values of a ligand for its target can both be determined by well-known methods, for example ELISA. Suitable assays to assess the EC50 and IC50 of polypeptides are set out in the Examples.

Part B2 of the polypeptide of the invention is a polypeptide binding domain specific for CTLA-4. This means that it binds to CTLA-4 preferably with a greater binding affinity than that at which it binds to another molecule. Part B2 preferably binds to CTLA-4 with with the same or with a higher affinity than that of wild-type human CD86 for CTLA-4.

Preferably, the Kd of part B2 of the polypeptide of the invention for human CTLA-4 will be at least 2-fold, at least 2.5-fold, at least 3-fold, at least 3.5-fold, at least 4-fold, at least 4.5-fold, at least 5-fold, at least 5.5-fold, at least 8-fold or at least 10-fold less than the Kd of wild-type human CD86 for human CTLA-4. Most preferably, the Kd of part B2 for human CTLA-4 will be at least 5-fold or at least 10-fold less than the Kd of wild-type human CD86 for human CTLA-4. A preferred method for determining the Kd of a polypeptide for CTLA-4 is SPR analysis, e.g. with a Biacore™ system. Suitable protocols for the SPR analysis of polypeptides are set out in the Examples.

Preferably, the EC50 of part B2 of the polypeptide of the invention for human CTLA-4 will be at least 1.5-fold, at least 2-fold, at least 3-fold, at least 5-fold, at least 10-fold, at least 12-fold, at least 14-fold, at least 15-fold, at least 17-fold, at least 20-fold, at least 25-fold or at least 50-fold less than the EC50 of wild-type human CD86 for human CTLA-4 under the same conditions. Most preferably, the EC50 of part B2 for human CTLA-4 will be at least 10-fold or at least 25-fold less than the EC50 of wild-type human CD86 for human CTLA-4 under the same conditions. A preferred method for determining the EC50 of a polypeptide for CTLA-4 is via ELISA. Suitable ELISA assays for use in the assessment of the EC50 of polypeptides are set out in the Examples.

Preferably, the IC50 of part B2 of the polypeptide of the invention when competing with wild-type human CD86 for binding to human CTLA-4 will be at least 2-fold, at least 3-fold, at least 4-fold, at least 5-fold, at least 10-fold, at least 13-fold, at least 15-fold, at least 50-fold, at least 100-fold, or at least 300-fold less than the IC50 of wild-type human CD86 under the same conditions. Most preferably, the IC50 of part B2 will be at least 10-fold or at least 300-fold less than the IC50 of wild-type human CD86 under the same conditions. A preferred method for determining the IC50 of a polypeptide of the invention is via ELISA. Suitable ELISA assays for use in the assessment of the IC50 of polypeptides of the invention are set out in the Examples.

Part B2 of the polypeptide of the invention may also bind specifically to CD28. That is, part B2 may bind to CD28 with greater binding affinity than that at which it binds to another molecule, with the exception of CTLA-4. Part B2 may bind to human CD28 with a lower affinity than that of wild-type human CD86 for human CD28. Preferably, the Kd of part B2 for human CD28 will be at least 2-fold, preferably at least 5-fold, more preferably at least 10-fold higher than the Kd of wild-type human CD86 for human CD28.

The binding properties of part B2 of the polypeptide of the invention may also be expressed as a relative measure of the ability of a polypeptide to bind to the two targets, CTLA-4 and CD28. That is, the binding properties of part B2 may be expressed as a relative measure of the ability of the polypeptide to bind to CTLA-4 versus its ability to bind to CD28. Preferably part B2 has an increased relative ability to bind to CTLA-4 versus CD28, when compared to the corresponding relative ability of human wild-type CD86 to bind to CTLA-4 versus CD28.

When the binding affinity of a polypeptide for both CTLA-4 and CD28 is assessed using the same parameter (e.g. Kd, EC50), then the relative binding ability of the polypeptide for each target may be expressed as a simple ratio of the values of the parameter for each target. This ratio may be referred to as the binding ratio or binding strength ratio of a polypeptide. For many parameters used to assess binding affinity (e.g. Kd, EC50), a lower value indicates a higher affinity. When this is the case, the ratio of binding affinities for CTLA-4 versus CD28 is preferably expressed as a single numerical value calculated according to the following formula:

Binding ratio=[binding affinity for CD28]÷[binding affinity for CTLA-4]

Alternatively, if binding affinity is assessed using a parameter for which a higher value indicates a higher affinity, the inverse of the above formula is preferred. In either context, part B2 of the polypeptide of the invention preferably has a higher binding ratio than human wild-type CD86. It will be appreciated that direct comparison of the binding ratio for a given polypeptide to the binding ratio for another polypeptide typically requires that the same parameters be used to assess the binding affinities and calculate the binding ratios for both polypeptides.

Preferably, the binding ratio for a polypeptide is calculated by determining the Kd of the polypeptide for each target and then calculating the ratio in accordance with the formula [Kd for CD28]÷[Kd for CTLA-4]. This ratio may be referred to as the Kd binding ratio of a polypeptide. A preferred method for determining the Kd of a polypeptide for a target is SPR analysis, e.g. with a Biacore™ system. Suitable protocols for the SPR analysis of polypeptides of the invention are set out in the Examples. The binding ratio of part B2 of the polypeptide of the invention calculated according to this method is preferably at least 2-fold or at least 4-fold higher than the binding ratio of wild-type human CD86 calculated according to the same method.

Alternatively, the binding ratio for a polypeptide may be calculated by determining the EC50 of the polypeptide for each target and then calculating the ratio in accordance with the formula [EC50 for CD28]÷[EC50 for CTLA-4]. This ratio may be referred to as the EC50 binding ratio of a polypeptide. A preferred method for determining the EC50 of a polypeptide for a target is via ELISA. Suitable ELISA assays for use in the assessment of the EC50 of polypeptides of the invention are set out in the Examples. The binding ratio of part B2 of the polypeptide of the invention calculated according to this method is at least 2-fold, at least 3-fold, at least 4-fold, at least 5-fold, at least 6-fold, at least 7-fold, at least 8-fold, at least 9-fold or at least 10-fold higher than the binding ratio of wild-type human CD86 calculated according to the same method.

Part B2 of the polypeptide of the invention may have the ability to cross-compete with another polypeptide for binding to CTLA-4. For example, part B2 may cross-compete with a polypeptide having the amino acid sequence of any one of SEQ ID NOs: 6 to 24 for binding to CTLA-4. Such cross-competing polypeptides may be identified in standard binding assays. For example, SPR analysis (e.g. with a Biacore™ system), ELISA assays or flow cytometry may be used to demonstrate cross-competition.

In addition to the above functional characteristics, part B2 of the polypeptide of the invention has certain preferred structural characteristics. Part B2 either comprises or consists of (i) the monomeric soluble extracellular domain of human wild-type CD86, or (ii) a polypeptide variant of said soluble extracellular domain, provided that said polypeptide variant binds to human CTLA-4 with higher affinity than wild-type human CD86.

A polypeptide variant of the monomeric soluble extracellular domain of human wild-type CD86 comprises or consists of an amino acid sequence which is derived from that of human wild-type CD86, specifically the amino acid sequence of the soluble extracellular domain of human wild-type CD86 (SEQ ID NO: 3), optionally lacking A24 and P25. In particular, a variant comprises an amino acid sequence in which at least one amino acid is changed when compared to the amino acid sequence of SEQ ID NO: 3 (or said sequence lacking A24 and P25). By “changed” it is meant that at least one amino acids is deleted, inserted, or substituted compared to the amino acid sequence of SEQ ID NO: 3 (or said sequence lacking A24 and P25). By “deleted” it is meant that the at least one amino acid present in the amino acid sequence of SEQ ID NO: 3 (or said sequence lacking A24 and P25) is removed, such that the amino acid sequence is shortened by one amino acid. By “inserted” it is meant that the at least one additional amino acid is introduced into the amino acid sequence of SEQ ID NO: 3 (or said sequence lacking A24 and P25), such that the amino acid sequence is lengthened by one amino acid. By “substituted” it is meant that the at least one amino acid in the amino acid sequence of SEQ ID NO: 3 (or said sequence lacking A24 and P25) is replaced with an alternative amino acid.

Amino acids herein may be referred to by full name, three letter code or single letter code, as set out below.

| | | |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Typtophan | Trp | W |
| Tyrosine | Tyr | Y |
| Valine | Val | V |

Typically, at least 1, 2, 3, 4, 5, 6, 7, 8 or 9 amino acids are changed when compared to the amino acid sequence of SEQ ID NO: 3 (or said sequence lacking A24 and P25). Typically, no more than 10, 9, 8, 7, 6, 5, 4, 2 or 1 amino acids are changed when compared to the amino acid sequence of SEQ ID NO: 3 (or said sequence lacking A24 and P25). It will be appreciated that any of these lower limits may be combined with any of these upper limits to define a range for the permitted number of changes compared to the amino acid sequence of SEQ ID NO: 3 (or said sequence lacking A24 and P25). Thus, for example, a polypeptide of the invention may comprise an amino acid sequence in which the permitted number of amino acid changes compared to the amino acid sequence of SEQ ID NO: 3 (or said sequence lacking A24 and P25) is in the range 2 to 3, 2 to 4, 2 to 5, 2 to 6, 2 to 7, 2 to 8, 2 to 9, 2 to 10, 3 to 4, 3 to 5, 3 to 6, and so on.

It is particularly preferred that at least 2 amino acids are changed when compared to the amino acid sequence of SEQ ID NO: 3 (or said sequence lacking A24 and P25). Preferably, the permitted number of amino acid changes compared to the amino acid sequence of SEQ ID NO: 3(or said sequence lacking A24 and P25) is in the range 2 to 9, 2 to 8 or 2 to 7.

The numbers and ranges set out above may be achieved with any combination of deletions, insertions or substitutions compared to the amino acid sequence of SEQ ID NO: 3 (or said sequence lacking A24 and P25). For example, there may be only deletions, only insertions, or only substitutions compared to the amino acid sequence of SEQ ID NO: 3 (or said sequence lacking A24 and P25), or any mixture of deletions, insertions or substitutions. Preferably the variant comprises an amino acid sequence in which all of the changes compared to the amino acid sequence of SEQ ID NO: 3 (or said sequence lacking A24 and P25) are substitutions. That is, a sequence in which no amino acids are deleted or inserted compared to the sequence of SEQ ID NO: 3 (or said sequence lacking A24 and P25). In the amino acid sequence of a preferred variant, 1, 2, 3, 4, 5, 6, 7, or 8 amino acids are substituted when compared to the amino acid sequence of SEQ ID NO: 3 (or said sequence lacking A24 and P25) and no amino acids are deleted or inserted compared to the sequence of SEQ ID NO: 3 (or said sequence lacking A24 and P25).

Preferably the changes compared to the sequence of SEQ ID NO: 3 (or said sequence lacking A24 and P25) are in the FG loop region (positions 114 to 121) and/or the beta sheet region of SEQ ID NO: 3. The strands of the beta sheet region have the following positions in SEQ ID NO: 3: A:27-31, B:36-37, C:54-58, C′:64-69, C″:72-74, D:86-88, E:95-97, F:107-113, G:122-133.

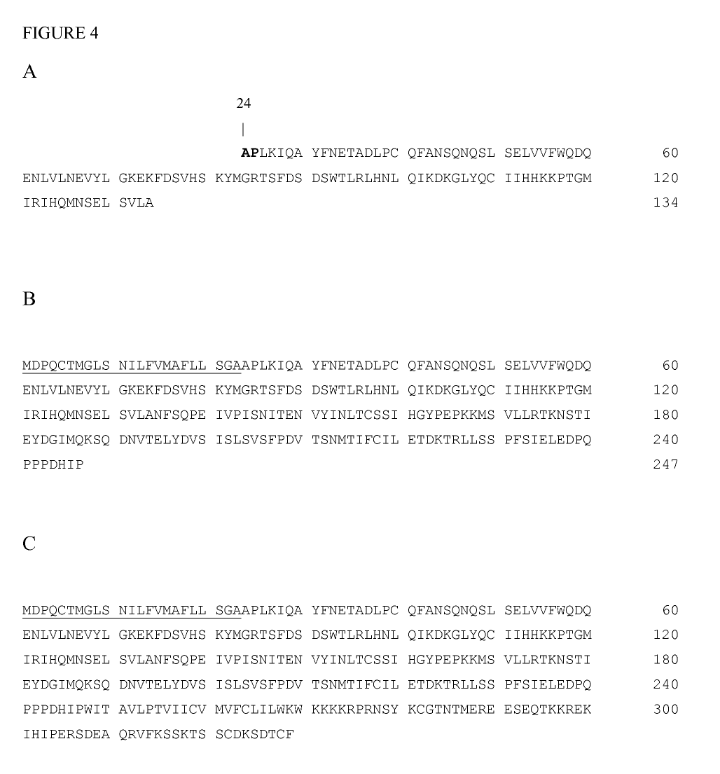

Most preferably, the changes compared to the sequence of SEQ ID NO: 3 (or said sequence lacking A24 and P25) are in one or more of the positions selected from 32, 48, 49, 54, 74, 77, 79, 103, 107, 111, 118, 120, 121, 122, 125, 127 or 134. All numbering of amino acid positions herein is based on counting the amino acids in SEQ ID NO: 4 starting from the N terminus. Thus, the first position at the N terminus of SEQ ID NO: 3 is numbered 24 (see schematic diagram in FIG. 4).

Particularly preferred insertions include a single additional amino acid inserted between positions 116 and 117 and/or a single additional amino acid inserted between positions 118 and 119. The inserted amino acid is preferably Tyrosine (Y), Serine (S), Glycine (G), Leucine (L) or Aspartic Acid (D).

A particularly preferred substitution is at position 122, which is Arginine (R). The polypeptide of the invention preferably includes an amino acid sequence in which position 122 is substituted compared to the amino acid sequence of SEQ ID NO: 3 (or said sequence lacking A24 and P25). The most preferred substitution at position 122 is to replace Arginine (R) with Lysine (K) or Asparagine (N), ranked in order of preference. This substitution may be referred to as R122K/N.

Other preferred substitutions are at positions 107, 121, and 125, which are Leucine (L), Isoleucine (I) and Glutamic acid (Q), respectively. In addition to the substitution at position 122, the polypeptide of the invention preferably includes an amino acid sequence in which at least one of the amino acids at positions 107, 121 and 125 is also substituted compared to the amino acid sequence of SEQ ID NO: 3 (or said sequence lacking A24 and P25). The amino acid sequence of the polypeptide of the invention may also be substituted at one or more of positions 32, 48, 49, 54, 64, 74, 77, 79, 103, 111, 118, 120, 127 and 134.

The most preferred substitution at position 107 is to replace Leucine (L) with Isoleucine(I), Phenylalanine(F) or Arginine(R), ranked in order of preference. This substitution may be referred to as L107I/F/R. Similar notation is used for other substitutions described herein. The most preferred substitution at position 121 is to replace Isoleucine (I) with Valine (V). This substitution may be referred to as I121V. The most preferred substitution at position 125 is to replace Glutamine (Q) with Glutamic acid (E). This substitution may be referred to as Q125E.

Other substitutions which may be preferred in the amino acid sequence of the polypeptide of the invention include: F32I, Q48L, S49T, V54I, V64I, K74I/R, S77A, H79D/S/A, K103E, I111V, T118S, M120L, N127S/D and A134T.

Particularly preferred variants of said soluble extracellular domain of human wild-type CD86 comprise or consist of any one of the following amino acid sequences of SEQ ID NOs: 6 to 24

| SEQ | | |
|---|---|---|
| ID | | |
| NO. | DESIGNATION | SEQUENCE |
| 6 | 900 | LKIQAYFNETADLPCQFANSQNQSLSELVVFWQDQENLVLNEVYLGKEKFDSVDSK |
| | | YMGRTSFDSDSWTLRLHNLQIKDKGIYQCVIHHKKPSGLVKIHEMNSELSVLA |
| 7 | 901 | LKIQAYFNETADLPCQFANSQNLTLSELVVFWQDQENLVLNEVYLGKEKFDSVHSK |
| | | YMGRTSFDSDSWTLRLHNLQIKDKGIYQCVIHHKKPTGMIKIHEMNSELSVLT |
| 8 | 904 | LKIQAYFNETADLPCQFANSQNQSLSELIVFWQDQENLVLNEVYLGKERFDAVDSK |
| | | YMGRTSFDSDSWTLRLHNLQIKDKGIYQCIIHHKKPSGMVKIHQMDSELSVLA |
| 9 | 906 | LKIQAYINETADLPCQFANSQNLSLSELVVFWQDQENLVLNEVYLGKERFDSVDSK |
| | | YMGRTSFDSDSWTLRLHNLQIKDKGFYQCIIHHKKPTGLVKIHEMNSELSVLA |
| 10 | 907 | LKIQAYFNETADLPCQFANSQNQSLSELVVFWQDQENLVLNEVYLGKEKFDSVHSK |
| | | YMGRTSFDSDSWTLRLHNLQIKDKGLYQCIIHHKKPTGMIKIHEMNSELSVLA |
| 11 | 908 | LKIQAYFNETADLPCQFANSQNQSLSELVVFWQDQENLVLNEVYLGKEKFDSVHSK |
| | | YMGRTSFDSDSWTLRLHNLQIKDKGIYQCIIHHKKPTGMVKIHEMNSELSVLA |
| 12 | 910 | LKIQAYFNETADLPCQFANSQNQSLSELVVFWQDQENLVLNEVYLGKEKFDSVDSK |
| | | YMGRTSFDSDSWTLRLHNLQIKDKGIYQCIIHHKKPTGMVKIHEMNSELSVLA |
| 13 | 915 | LKIQAYFNETADLPCQFANSQNQSLSELVVFWQDQENLILNEVYLGKEKFDSVDSK |
| | | YMGRTSFDSDSWTLRLHNLQIKDKGFYQCIIHHKKPSGLIKIHQMDSELSVLA |
| 14 | 938 | LKIQAYFNETADLPCQFANSQNQSLSELVVFWQDQENLILNEVYLGKEKFDSVHSK |
| | | YMGRTSFDSDSWTLRLHNLQIKDKGIYQCIIHHKKPTGMVKIHQMNSELSVLA |
| 15 | 1038 | APLKIQAYFNETADLPCQFANSQNLSLSELVVFWQDQENLVLNEVYLGKEKFDSVD |
| | | SKYMGRTSFDSDSWTLRLHNLQIKDKGIYQCIIHHKKPTGMVKIHEMNSELSVLA |
| 16 | 1039 | APLKIQAYFNETADLPCQFANSQNLSLSELVVFWQDQENLVLNEVYLGKEKFDSVS |
| | | SKYMGRTSFDSDSWTLRLHNLQIKDKGIYQCIIHHKKPSGMVKIHQMDSELSVLA |
| 17 | 1040 | APLKIQAYFNETADLPCQFANSQNLSLSELVVFWQDQENLVLNEVYLGKERFDSVD |
| | | SKYMGRTSFDSDSWTLRLHNLQIKDKGRYQCIIHHKKPTGMINIHQMNSELSVLA |
| 18 | 1041 | APLKIQAYLNETADLPCQFANSQNLSLSELVVFWQDQENLVLNEVYLGKEKFDSVD |
| | | SKYMGRTSFDSDSWTLRLHNLQIKDKGIYQCIIHHKKPTGLVKIHEMNSELSVLA |
| 19 | 1042 | APLKIQAYFNETADLPCQFANSQNLSLSELVVFWQDQENLVLNEVYLGKEIFDSVS |
| | | SKYMGRTSFDSDSWTLRLHNLQIKDKGIYQCIIHHKKPSGMVKIHQMDSELSVLA |
| 20 | 1043 | APLKIQAYFNETADLPCQFANSQNLSLSELVVFWQDQENLVLNEVYLGKEKFDSVD |
| | | SKYMGRTSFDSDSWTLRLHNLQIKDKGIYQCIIHHKKPTGMIKIHEMNSELSVLA |
| 21 | 1044 | APLKIQAYFNETADLPCQFANSQNLTLSELVVFWQDQENLVLNEVYLGKEKFDSVS |
| | | SKYMGRTSFDSDSWTLRLHNLQIKDKGIYQCIIHHKKPTGMIKIHEMSSELSVLA |
| 22 | 1045 | APLKIQAYFNETADLPCQFANSQNLTLSELVVFWQDQENLVLNEVYLGKEKFDSVD |
| | | SKYMGRTSFDSDSWTLRLHNLQIKDKGLYQCIIHHKKPTGLVKIHEMNSELSVLA |
| 23 | 1046 | APLKIQAYFNETADLPCQFANSQNQSLSELVVFWQDQENLVLNEVYLGKEKFDSVD |
| | | SKYMGRTSFDSDSWTLRLHNLQIEDKGIYQCIIHHKKPSGMVKIHQMDSELSVLA |
| 24 | 1047 | APLKIQAYFNETADLPCQFANSQNLSLSELVVFWQDQENLVLNEVYLGKEKFDSVD |
| | | SKYMGRTSFDSDSWTLRLHNLQIKDKGIYQCIIHHKKPTGLVKIHEMNSELSVLA |

The amino acid sequences shown in SEQ ID NOs: 6 to 14 may optionally include the additional residues AP at the N-terminus . The amino acid sequences shown in SEQ ID NOs: 15 to 24 may optionally lack the residues AP at the N-terminus. In either case, these residues correspond to A24 and P25 of SEQ ID NO: 3.

Part B2 of the polypeptide of the invention may comprise or consist of any of the above-described variants of said soluble extracellular domain of human wild-type CD86. That is, part B2 of the polypeptide of the invention may comprise or consist of the amino acid sequence of any one of SEQ ID NOs: 6 to 24.

The binding domain may modulate signalling from CTLA-4, for example when administered to a cell expressing CTLA-4, such as a T cell. Preferably the binding domain reduces, i.e. inhibits or blocks, said signalling and thereby increases the activation of said cell. Changes in CTLA-4 signalling and cell activation as a result of administration of a test agent (such as the binding domain) may be determined by any suitable method. Suitable methods include assaying for the ability of membrane-bound CD86 (e.g. on Raji cells) to bind and signal through CTLA-4 expressed on the surface of

T cells, when in the presence of a test agent or in the presence of a suitable control. An increased level of T cell IL-2 production or an increase in T cell proliferation in the presence of the test agent relative to the level of T cell IL-2 production and/or T cell proliferation in the presence of the control is indicative of reduced signalling through CTLA-4 and increased cell activation. A typical assay of this type is disclosed in Example 9 of US20080233122.

### The Polypeptide of the Invention

The polypeptide of the invention is capable of specifically binding to both human CTLA-4 and human CD40, and comprises B1 and B2, wherein: B1 is an antibody, or antigen binding fragment thereof, specific for human CD40; and

B2 is a polypeptide binding domain specific for human CTLA-4, which comprises or consists of:
(i) the amino acid sequence of SEQ ID NO: 3; or
(ii) an amino acid sequence in which at least one amino acid is changed when compared to the amino acid sequence of SEQ ID NO: 3 provided that said binding domain binds to human CTLA-4 with higher affinity than wild-type human CD86.
   By capable of specifically binding to both CTLA-4 and CD40, it is meant that part B1 specifically binds to CD40 and part B2 specifically binds to CTLA-4, in accordance with the definitions provided for each part above. Preferably the binding characteristics of parts B1 and B2 for their respective targets are unchanged or substantially unchanged when they are present as part of a polypeptide of the invention, when compared to said characteristics for parts B1 and B2 when present as separate entities. The binding characteristics of parts B1 and B2 when present as part a polypeptide of the invention may be assessed by any suitable assay. In particular, the assays set out above for each separate part may also be applied to B1 and B2 when they are present as part of a polypeptide of the invention. Suitable assays for assessed the binding characteristics of polypeptides of the invention are also set out in the Examples.

Part B1 of the polypeptide of the invention is an antibody, or antigen-binding fragment thereof, which typically comprises at least one heavy chain (H) and/or at least one light chain (L). Part B2 of the polypeptide of the invention may be attached to any part of B1, but may typically be attached to said at least one heavy chain (H) or least one light chain (L), preferably at either the N or the C terminus. Part B2 of the polypeptide of the invention may be so attached either directly or indirectly via any suitable linking molecule (a linker).

Part B1 preferably comprises at least one heavy chain (H) and at least one light chain (L) and part B2 is preferably attached to the N or the C terminus of either said heavy chain (H) or said light chain (L). An exemplary antibody of B1 consists of two identical heavy chains (H) and two identical light chains (L). Such an antibody is typically arranged as two arms, each of which has one H and one L joined as a heterodimer, and the two arms are joined by disulfide bonds between the H chains. Thus, the antibody is effectively a homodimer formed of two H-L heterodimers. Part B2 of the polypeptide of the invention may be attached to both H chains or both L chains of such an antibody, or to just one H chain, or just one L chain.

The polypeptide of the invention may therefore alternatively be described as an anti-CD40 antibody, or an antigen binding fragment thereof, to which is attached at least one polypeptide binding domain specific for CTLA-4, which comprises or consists of the monomeric soluble extracellular domain of human wild-type CD86 or a variant thereof The binding domains of B1 and B2 may be the only binding domains in the polypeptide of the invention.

The polypeptide of the invention may comprise a polypeptide arranged according to any one of the following formulae, written in the direction N-C:

H-(X)n-B2; (a)

B2-(X)n-H; (b)

L-(X)n-B2; or (c)

B2-(X)n-L (d)

wherein H is the heavy chain of an antibody (i.e. of B1), L is the light chain of an antibody (i.e. of B1), Xis a linker and n is 0 or 1. Where the linker (X) is a peptide, it typically has the amino acid sequence SGGGGSGGGGS, SGGGGSGGGGSAP, NFSQP, KRTVA or (SG)m, where m =1 to 7.

The present invention also provides a polypeptide which consists of a polypeptide arranged according to any of formulae (a) to (d). Said polypeptide may be provided as a monomer or may be present as a component of a multimeric protein, such as an antibody. Said polypeptide may be isolated. Examples of such polypeptides are provided as SEQ ID NOs: 56 to 60 and 110 to 114.

Part B2 may be attached to any part of a polypeptide of the invention, or to a linker, by any suitable means. For example, the various parts of the polypeptide may be joined by chemical conjugation, such as with a peptide bond. Thus the polypeptide of the invention may comprise or consist of a fusion protein comprising B1 (or a component part therof) and B2, optionally joined by a peptide linker. In such a fusion protein, the CD40-binding domain or domains of B1 and the CTLA-4-binding domain or domains of B2 may be the only binding domains.

Other methods for conjugating molecules to polypeptides are known in the art. For example, carbodiimide conjugation (Bauminger & Wilchek (1980) Methods Enzymol. 70, 151-159) may be used to conjugate a variety of agents, including doxorubicin, to antibodies or peptides. The water-soluble carbodiimide, 1-ethyl-3-(3-dimethylaminopropyl) carbodiimide (EDC) is particularly useful for conjugating a functional moiety to a binding moiety. As a further example, conjugation may be achieved by sodium periodate oxidation followed by reductive alkylation of appropriate reactants, or by glutaraldehyde cross-linking However, it is recognised that, regardless of which method is selected, a determination should preferably be made that parts B1 and B2 retain or substantially retain their target binding properties when present as parts of the polypeptide of the invention.

The same techniques may be used to link the polypeptide of the invention(directly or indirectly) to another molecule. The other molecule may be a a therapeutic agent or a detectable label. Suitable therapeutic agents include a cytotoxic moiety or a drug.

A polypeptide of the invention may be provided in isolated or substantially isolated form. By substantially isolated, it is meant that there may be substantial, but not total, isolation of the polypeptide from any surrounding medium. The polypeptides may be mixed with carriers or diluents which will not interfere with their intended use and still be regarded as substantially isolated.

Exemplary polypeptides of the invention may comprise or consist of the amino acid sequence of any one of SEQ ID NOs: 56 to 60 as shown below.

| SEQ | |
|---|---|
| ID | |
| 56 | Bispecific polypeptide 956/530 |
| | QSVLTQPPSASGTPGQRVTISCSGSSSNIGSNTVNWYQQLPGTAPKLLIYRNNQRPSGVPDRFSG |
| | SKSGTSASLAISGLRSEDEADYYCAAWDDSLSGWRFGGGTKLTVLGGTVAAPSVFIFPPSDEQLK |
| | SGTASVVCLLNNFYPREAKVQWKVDNALQSGNSQESVTEQDSKDSTYSLSSTLTLSKADYEKHKV |
| | YACEVTHQGLSSPVTKSFNRGECSGGGGSGGGGSLKIQAYFNETADLPCQFANSQNQSLSELIVF |
| | WQDQENLVLNEVYLGKERFDAVDSKYMGRTSFDSDSWTLRLHNLQIKDKGIYQCIIHHKKPSGMV |
| | KIHQMDSELSVLA |
| | A2-54 light chain, VL-kappa |
| | Linker (underlined) SGGGGSGGGGS |
| | 904: CD86 mutant molecule (bold) |
| | ANTIBODY PREFERBALY ASSEMBLES WITH A2-54 heavy chain |
| 57 | Bispecific polypeptide 957/530 |
| | QSVLTQPPSASGTPGQRVTISCSGSSSNIGSNTVNWYQQLPGTAPKLLIYRNNQRPSGVPDRFSG |
| | SKSGTSASLAISGLRSEDEADYYCAAWDDSLSGWRFGGGTKLTVLGGTVAAPSVFIFPPSDEQLK |
| | SGTASVVCLLNNFYPREAKVQWKVDNALQSGNSQESVTEQDSKDSTYSLSSTLTLSKADYEKHKV |
| | YACEVTHQGLSSPVTKSFNRGECSGGGGSGGGGSAPLKIQAYFNETADLPCQFANSQNQSLSELI |
| | VFWQDQENLVLNEVYLGKERFDAVDSKYMGRTSFDSDSWTLRLHNLQIKDKGIYQCIIHHKKPSG |
| | MVKIHQMDSELSVLA |
| | A2-54 light chain, VL-kappa |
| | linker: (underlined) SGGGGSGGGGS |
| | Amino acids inserted: AP |
| | 904: CD86 mutant molecule (bold) |
| | ANTIBODY PREFERBALY ASSEMBLES WITH A2-54 heavy chain |
| 58 | Bispecific polypeptide 958/531 |
| | LKIQAYFNETADLPCQFANSQNQSLSELIVFWQDQENLVLNEVYLGKERFDAVDSKYMGRTSFDS |
| | DSWTLRLHNLQIKDKGIYQCIIHHKKPSGMVKIHQMDSELSVLANFSQPEVQLLESGGGLVQPGG |
| | SLRLSCAASGFTFSDYYMSWVRQAPGKGLEWISSISSNGIYIYYADSLKGRFTISRDNSKNTLYL |
| | QMNSLRAEDTAVYYCARAPVDYSNPSGMDVWGQGTLVTVSSASTKGPSVFPLAPSSKSTSGGTAA |
| | LGCLVKDYFPEPVTVSWNSGALTSGVHTFPAVLQSSGLYSLSSVVTVPSSSLGTQTYICNVNHKP |
| | SNTKVDKKVEPKSCDKTHTCPPCPAPELLGGPSVFLFPPKPKDTLMISRTPEVTCVVVDVSHEDP |
| | EVKFNWYVDGVEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPGK |
| | 904: mutant CD86 molecule (bold) |
| | linker: (underlined) NFSQP |
| | A2-54 Heavy Chain, VH-gamma1 |
| | ANTIBODY PREFERBALY ASSEMBLES WITH A2-54 light chain |
| 59 | Bispecific polypeptide 959/530 |
| | LKIQAYFNETADLPCQFANSQNQSLSELIVFWQDQENLVLNEVYLGKERFDAVDSKYMGRTSFDS |
| | DSWTLRLHNLQIKDKGIYQCIIHHKKPSGMVKIHQMDSELSVLAKRTVAQSVLTQPPSASGTPGQ |
| | RVTISCSGSSSNIGSNTVNWYQQLPGTAPKLLIYRNNQRPSGVPDRFSGSKSGTSASLAISGLRS |
| | EDEADYYCAAWDDSLSGWRFGGGTKLTVLGGTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTK |
| | SFNRGEC |
| | 904: mutant CD86 molecule (bold) |
| | Linker: (underlined) KRTVA |
| | A2-54 Variable Light chain, VL-kappa |
| | ANTIBODY PREFERBALY ASSEMBLES WITH A2-54 heavy chain |
| 60 | Bispecific polypeptide 960/530 |
| | LKIQAYFNETADLPCQFANSQNQSLSELIVFWQDQENLVLNEVYLGKERFDAVDSKYMGRTSFDS |
| | DSWTLRLHNLQIKDKGIYQCIIHHKKPSGMVKIHQMDSELSVLANFSQPQSVLTQPPSASGTPGQ |
| | RVTISCSGSSSNIGSNTVNWYQQLPGTAPKLLIYRNNQRPSGVPDRFSGSKSGTSASLAISGLRS |
| | EDEADYYCAAWDDSLSGWRFGGGTKLTVLGGTVAAPSVFIFPPSDEQLKSGTASVVCLLNNFYPR |
| | EAKVQWKVDNALQSGNSQESVTEQDSKDSTYSLSSTLTLSKADYEKHKVYACEVTHQGLSSPVTK |
| | SFNRGEC |
| | 904: mutant CD86 molecule (bold) |
| | linker: (underlined) NFSQP |
| | A2-54 Variable Light chain, VL-kappa |
| | ANTIBODY PREFERBALY ASSEMBLES WITH A2-54 heavy chain |
| 110 | Bispecific polypeptide 1107/1145 |
| | DIQMTQSPSSLSASVGDRVTITCRASQSISSYLNWYQQKPGKAPKLLIYAASSLQSGVPSRFSGS |
| | GSGTDFTLTISSLQPEDFATYYCQQYGVYPFTFGQGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVYACE |
| | VTHQGLSSPVTKSFNRGECSGGGGSGGGGSAPLKIQAYFNETADLPCQFANSQNLSLSELVVFWQ |
| | DQENLVLNEVYLGKERFDSVDSKYMGRTSFDSDSWTLRLHNLQIKDKGRYQCIIHHKKPTGMINI |
| | HQMNSELSVLA |
| | clone 1145 light chain, VL-kappa |
| | Linker (underlined) SGGGGSGGGGS |
| | 1040: CD86 mutant molecule (bold) |
| | ANTIBODY PREFERBALY ASSEMBLES WITH HEAVY CHAIN 1107 |
| 111 | Bispecific polypeptide 1136/1143 |
| | DIQMTQSPSSLSASVGDRVTITCRASQSISSYLNWYQQKPGKAPKLLIYAASSLQSGVPSRFSGS |
| | GSGTDFTLTISSLQPEDFATYYCQQAYYAGLFTFGQGTKLEIKRTVAAPSVFIFPPSDEQLKSGT |
| | ASVVCLLNNFYPREAKVQWKVDNALQSGNSQESVTEQDSKDSTYSLSSTLTLSKADYEKHKVYAC |
| | EVTHQGLSSPVTKSFNRGECSGGGGSGGGGSAPLKIQAYFNETADLPCQFANSQNLSLSELVVEW |
| | QDQENLVLNEVYLGKERFDSVDSKYMGRTSFDSDSWTLRLHNLQIKDKGRYQCIIHHKKPTGMIN |
| | IHQMNSELSVLA |
| | clone 1143 light chain, VL-kappa |
| | Linker (underlined)SGGGGSGGGGS |
| | Amino acids inserted: AP |
| | 1040: CD86 mutant molecule (bold) |
| | ANTIBODY PREFERBALY ASSEMBLES WITH HEAVY CHAIN 1136 |
| 112 | Bispecific polypeptide 1132/1139 |
| | DIQMTQSPSSLSASVGDRVTITCRASQSISSYLNWYQQKPGKAPKLLIYAASSLQSGVPSRFSGS |
| | GSGTDFTLTISSLQPEDFATYYCQQYGRNPPTFGQGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVYACE |
| | VTHQGLSSPVTKSFNRGECSGGGGSGGGGSAPLKIQAYFNETADLPCQFANSQNLSLSELVVFWQ |
| | DQENLVLNEVYLGKERFDSVDSKYMGRTSFDSDSWTLRLHNLQIKDKGRYQCIIHHKKPTGMINI |
| | HQMNSELSVLA |
| | clone 1139 light chain, VL-kappa |
| | Linker (underlined)SGGGGSGGGGS |
| | Amino acids inserted: AP |
| | 1040: CD86 mutant molecule (bold) |
| | ANTIBODY PREFERBALY ASSEMBLES WITH HEAVY CHAIN 1132 |
| 113 | Bispecific polypeptides 1140/1141 and 1134/1141 |
| | DIQMTQSPSSLSASVGDRVTITCRASQSISSYLNWYQQKPGKAPKLLIYAASSLQSGVPSRFSGS |
| | GSGTDFTLTISSLQPEDFATYYCQQSYSTPYTFGQGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVYACE |
| | VTHQGLSSPVTKSFNRGECSGGGGSGGGGSAPLKIQAYFNETADLPCQFANSQNLSLSELVVFWQ |
| | DQENLVLNEVYLGKERFDSVDSKYMGRTSFDSDSWTLRLHNLQIKDKGRYQCIIHHKKPTGMINI |
| | HQMNSELSVLA |
| | clone 1141 light chain, VL-kappa |
| | Linker (underlined)SGGGGSGGGGS |
| | Amino acids inserted: AP |
| | 1040: CD86 mutant molecule (bold) |
| | FOR 1140/1141: ANTIBODY ASSEMBLES WITH HEAVY CHAIN 1140 |
| | FOR 1134/1141: ANTIBODY ASSEMBLES WITH HEAVY CHAIN 1134 |
| 114 | 1150/1152 |
| | DIQMTQSPSSLSASVGDHVTITCRASQSISSYLNWYQQKPGKAPKLLIYAASSLQSGVPSRFSGS |
| | GSGTDFTLTISSLQPEDFATYYCQQYGSAPPTFGQGTKLEIKRTVAAPSVFIFPPSDEQLKSGTA |
| | SVVCLLNNFYPREAKVQWKVDNALQSGNSQESVTEQDSKDSTYSLSSTLTLSKADYEKHKVYACE |
| | VTHQGLSSPVTKSFNRGECSGGGGSGGGGSAPLKIQAYFNETADLPCQFANSQNLSLSELVVFWQ |
| | DQENLVLNEVYLGKERFDSVDSKYMGRTSFDSDSWTLRLHNLQIKDKGRYQCIIHHKKPTGMINI |
| | HQMNSELSVLA |
| | clone 1152 light chain, VL-kappa |
| | Linker (underlined) SGGGGSGGGGS |
| | Amino acids inserted: AP |
| | 1040: CD86 mutant molecule (bold) |
| | ANTIBODY PREFERABLY ASSEMBLES WITH HEAVY CHAIN 1150 |

The polypeptide of the invention may be produced by any suitable means. For example, all or part of the polypeptide may be expressed as a fusion protein by a cell comprising a nucleotide which encodes said polypeptide.

Alternatively parts B1 and B2 may be produced separately and then subsequently joined together. Joining may be achieved by any suitable means, for example using the chemical conjugation methods and linkers outlined above. Separate production of parts B1 and B2 may be achieved by any suitable means. For example by expression from separate nucleotides optionally in separate cells, as is explained in more detail below.

### Polynucleotides, Vectors and Cells

The invention also relates to polynucleotides that encode all or part of a polypeptide of the invention . Thus, a polynucleotide of the invention may encode any polypeptide as described herein, or all or part of B1 or all or part of B2. The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Representative polynucleotides which encode examples of a heavy chain or light chain amino acid sequence of an antibody may comprise or consist of any one of the sequences set out below.

SEQ
ID

Antibody A2-54

| | | |
|---|---|---|
| 63 | A2-54 | GAGGTGCAGCTGCTGGAGTCCGGAGGAGGCCTGGTGCAGCCCGGCGGCAGCCTGAGAC |
| | heavy | TGAGCTGTGCCGCCAGCGGATTCACCTTCTCCGACTACTATATGAGCTGGGTGAGGCA |
| | chain, | GGCCCCAGGCAAGGGCCTGGAGTGGATTTCCTCCATCAGCAGCAATGGGATCTACATT |
| | VH- | TACTACGCCGACAGCCTGAAGGGCAGGTTCACAATCAGCAGGGACAACTCTAAGAACA |
| | gamma1 | CACTGTACCTGCAGATGAACTCCCTGCGCGCCGAGGACACCGCCGTGTATTACTGTGC |
| | | CAGGGCCCCCGTGGACTACTCTAATCCCAGCGGCATGGACGTGTGGGGCCAGGGCACC |
| | | CTGGTGACAGTGAGCTCAGGTGAGTCGTACGCTAGCAAGCTTTCTGGGGCAGGCCAGG |
| | | CCTGACCTTGGCTTTGGGGCAGGGAGGGGGCTAAGGTGAGGCAGGTGGCGCCAGCCAG |
| | | GTGCACACCCAATGCCCATGAGCCCAGACACTGGACGCTGAACCTCGCGGACAGTTAA |
| | | GAACCCAGGGGCCTCTGCGCCCTGGGCCCAGCTCTGTCCCACACCGCGGTCACATGGC |
| | | ACCACCTCTCTTGCAGCCTCCACCAAGGGCCCATCGGTCTTCCCCCTGGCACCCTCCT |
| | | CCAAGAGCACCTCTGGGGGCACAGCGGCCCTGGGCTGCCTGGTCAAGGACTACTTCCC |
| | | CGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCT |
| | | CCAGCAGCTTGGGCACCCAGACCTACATCTGCAACGTGAATCACAAGCCCAGCAACAC |
| | | CAAGGTGGACAAGAAAGTTGGTGAGAGGCCAGCACAGGGAGGGAGGGTGTCTGCTGGA |
| | | AGCCAGGCTCAGCGCTCCTGCCTGGACGCATCCCGGCTATGCAGCCCCAGTCCAGGGC |
| | | AGCAAGGCAGGCCCCGTCTGCCTCTTCACCCGGAGGCCTCTGCCCGCCCCACTCATGC |
| | | TCAGGGAGAGGGTCTTCTGGCTTTTTCCCCAGGCTCTGGGCAGGCACAGGCTAGGTGC |
| | | CCCTAACCCAGGCCCTGCACACAAAGGGGCAGGTGCTGGGCTCAGACCTGCCAAGAGC |
| | | CATATCCGGGAGGACCCTGCCCCTGACCTAAGCCCACCCCAAAGGCCAAACTCTCCAC |
| | | TCCCTCAGCTCGGACACCTTCTCTCCTCCCAGATTCCAGTAACTCCCAATCTTCTCTC |
| | | TGCAGAGCCCAAATCTTGTGACAAAACTCACACATGCCCACCGTGCCCAGGTAAGCCA |
| | | GCCCAGGCCTCGCCCTCCAGCTCAAGGCGGGACAGGTGCCCTAGAGTAGCCTGCATCC |
| | | AGGGACAGGCCCCAGCCGGGTGCTGACACGTCCACCTCCATCTCTTCCTCAGCACCTG |
| | | AACTCCTGGGGGGACCGTCAGTCTTCCTCTTCCCCCCAAAACCCAAGGACACCCTCAT |
| | | GATCTCCCGGACCCCTGAGGTCACATGCGTGGTGGTGGACGTGAGCCACGAAGACCCT |
| | | GAGGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTGCA |
| | | CCAGGACTGGCTGAATGGCAAGGAGTACAAGTGCAAGGTCTCCAACAAAGCCCTCCCA |
| | | GCCCCCATCGAGAAAACCATCTCCAAAGCCAAAGGTGGGACCCGTGGGGTGCGAGGGC |
| | | CACATGGACAGAGGCCGGCTCGGCCCACCCTCTGCCCTGAGAGTGACCGCTGTACCAA |
| | | CCTCTGTCCCTACAGGGCAGCCCCGAGAACCACAGGTGTACACCCTGCCCCCATCCCG |
| | | GGATGAGCTGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTACAGCAAGCTCACCGTGGA |
| | | CAAGAGCAGGTGGCAGCAGGGGAACGTCTTCTCATGCTCCGTGATGCATGAGGCTCTG |
| | | CACAACCACTACACGCAGAAGAGCCTCTCCCTGTCTCCGGGTAAATGA |
| | | GAGGTGCAGCTGCTGGAGTCCGGAGGAGGCCTGGTGCAGCCCGGCGGCAGCCTGAGAC |
| | | TGAGCTGTGCCGCCAGCGGATTCACCTTCTCCGACTACTATATGAGCTGGGTGAGGCA |
| | | GGCCCCAGGCAAGGGCCTGGAGTGGATTTCCTCCATCAGCAGCAATGGGATCTACATT |
| | | TACTACGCCGACAGCCTGAAGGGCAGGTTCACAATCAGCAGGGACAACTCTAAGAACA |
| | | CACTGTACCTGCAGATGAACTCCCTGCGCGCCGAGGACACCGCCGTGTATTACTGTGC |
| | | CAGGGCCCCCGTGGACTACTCTAATCCCAGCGGCATGGACGTGTGGGGCCAGGGCACC |
| | | CTGGTGACAGTGAGCTCAGGTGAGTCGTACGCTAGCAAGCTTTCTGGGGCAGGCCAGG |
| | | CCTGACCTTGGCTTTGGGGCAGGGAGGGGGCTAAGGTGAGGCAGGTGGCGCCAGCCAG |
| | | GTGCACACCCAATGCCCATGAGCCCAGACACTGGACGCTGAACCTCGCGGACAGTTAA |
| | | GAACCCAGGGGCCTCTGCGCCCTGGGCCCAGCTCTGTCCCACACCGCGGTCACATGGC |
| | | ACCACCTCTCTTGCAGCCTCCACCAAGGGCCCATCGGTCTTCCCCCTGGCACCCTCCT |
| | | CCAAGAGCACCTCTGGGGGCACAGCGGCCCTGGGCTGCCTGGTCAAGGACTACTTCCC |
| | | CGAACCGGTGACGGTGTCGTGGAACTCAGGCGCCCTGACCAGCGGCGTGCACACCTTC |
| | | CCGGCTGTCCTACAGTCCTCAGGACTCTACTCCCTCAGCAGCGTGGTGACCGTGCCCT |
| | | CCAGCAGCTTGGGCACCCAGACCTACATCTGCAACGTGAATCACAAGCCCAGCAACAC |
| | | CAAGGTGGACAAGAAAGTTGGTGAGAGGCCAGCACAGGGAGGGAGGGTGTCTGCTGGA |
| | | AGCCAGGCTCAGCGCTCCTGCCTGGACGCATCCCGGCTATGCAGCCCCAGTCCAGGGC |
| | | AGCAAGGCAGGCCCCGTCTGCCTCTTCACCCGGAGGCCTCTGCCCGCCCCACTCATGC |
| | | TCAGGGAGAGGGTCTTCTGGCTTTTTCCCCAGGCTCTGGGCAGGCACAGGCTAGGTGC |
| | | CCCTAACCCAGGCCCTGCACACAAAGGGGCAGGTGCTGGGCTCAGACCTGCCAAGAGC |
| | | CATATCCGGGAGGACCCTGCCCCTGACCTAAGCCCACCCCAAAGGCCAAACTCTCCAC |
| | | TCCCTCAGCTCGGACACCTTCTCTCCTCCCAGATTCCAGTAACTCCCAATCTTCTCTC |
| | | TGCAGAGCCCAAATCTTGTGACAAAACTCACACATGCCCACCGTGCCCAGGTAAGCCA |
| | | GCCCAGGCCTCGCCCTCCAGCTCAAGGCGGGACAGGTGCCCTAGAGTAGCCTGCATCC |
| | | AGGGACAGGCCCCAGCCGGGTGCTGACACGTCCACCTCCATCTCTTCCTCAGCACCTG |
| | | AACTCCTGGGGGGACCGTCAGTCTTCCTCTTCCCCCCAAAACCCAAGGACACCCTCAT |
| | | GATCTCCCGGACCCCTGAGGTCACATGCGTGGTGGTGGACGTGAGCCACGAAGACCCT |
| | | GAGGTCAAGTTCAACTGGTACGTGGACGGCGTGGAGGTGCATAATGCCAAGACAAAGC |
| | | CGCGGGAGGAGCAGTACAACAGCACGTACCGTGTGGTCAGCGTCCTCACCGTCCTGCA |
| | | CCAGGACTGGCTGAATGGCAAGGAGTACAAGTGCAAGGTCTCCAACAAAGCCCTCCCA |
| | | GCCCCCATCGAGAAAACCATCTCCAAAGCCAAAGGTGGGACCCGTGGGGTGCGAGGGC |
| | | CACATGGACAGAGGCCGGCTCGGCCCACCCTCTGCCCTGAGAGTGACCGCTGTACCAA |
| | | CCTCTGTCCCTACAGGGCAGCCCCGAGAACCACAGGTGTACACCCTGCCCCCATCCCG |
| | | GGATGAGCTGACCAAGAACCAGGTCAGCCTGACCTGCCTGGTCAAAGGCTTCTATCCC |
| | | AGCGACATCGCCGTGGAGTGGGAGAGCAATGGGCAGCCGGAGAACAACTACAAGACCA |
| | | CGCCTCCCGTGCTGGACTCCGACGGCTCCTTCTTCCTCTACAGCAAGCTCACCGTGGA |
| | | CAAGAGCAGGTGGCAGCAGGGGAACGTCTTCTCATGCTCCGTGATGCATGAGGCTCTG |
| | | CACAACCACTACACGCAGAAGAGCCTCTCCCTGTCTCCGGGTAAATGA |
| 64 | A2-54 | CAGTCCGTGCTGACCCAGCCACCCTCCGCCAGCGGCACCCCTGGCCAGCGGGTGACCA |
| | light | TCTCTTGTAGCGGCAGCAGCTCCAACATCGGAAGCAATACCGTGAATTGGTACCAGCA |
| | chain, | GCTGCCCGGCACCGCCCCCAAGCTGCTGATCTACCGCAATAACCAGAGGCCCTCCGGC |
| | VL-kappa | GTGCCCGACAGGTTCAGCGGCAGCAAGTCTGGCACCTCCGCCTCTCTGGCCATTAGCG |
| | | GACTGCGGAGCGAGGACGAGGCCGATTACTACTGCGCCGCCTGGGACGACTCCCTGTC |
| | | CGGGTGGCGCTTTGGAGGCGGCACAAAGCTGACCGTGCTGGGAGGTGAGTAGAACGTA |
| | | CGCTAGCAAGCTTGATATCGAATTCTAAACTCTGAGGGGGTCGGATGACGTGGCCATT |
| | | CTTTGCCTAAAGCATTGAGTTTACTGCAAGGTCAGAAAAGCATGCAAAGCCCTCAGAA |
| | | TGGCTGCAAAGAGCTCCAACAAAACAATTTAGAACTTTATTAAGGAATAGGGGGAAGC |
| | | TAGGAAGAAACTCAAAACATCAAGATTTTAAATACGCTTCTTGGTCTCCTTGCTATAA |
| | | TTATCTGGGATAAGCATGCTGTTTTCTGTCTGTCCCTAACATGCCCTGTGATTATCCG |
| | | CAAACAACACACCCAAGGGCAGAACTTTGTTACTTAAACACCATCCTGTTTGCTTCTT |
| | | TCCTCAGGAACTGTGGCTGCACCATCTGTCTTCATCTTCCCGCCATCTGATGAGCAGT |
| | | TGAAATCTGGAACTGCCTCTGTTGTGTGCCTGCTGAATAACTTCTATCCCAGAGAGGC |
| | | CAAAGTACAGTGGAAGGTGGATAACGCCCTCCAATCGGGTAACTCCCAGGAGAGTGTC |
| | | ACAGAGCAGGACAGCAAGGACAGCACCTACAGCCTCAGCAGCACCCTGACGCTGAGCA |
| | | AAGCAGACTACGAGAAACACAAAGTCTACGCCTGCGAAGTCACCCATCAGGGCCTGAG |
| | | CTCGCCCGTCACAAAGAGCTTCAACAGGGGAGAGTGT |

1107/1145

| | | |
|---|---|---|
| 90 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1107, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCCGTGTTTGGGGTTTTGACTATTGGGGCCAGGGAACCCTGGTCACCGTCTCCTCA |
| 91 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1145, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGTACGGTGTTTACCCGTTCAC |
| | VL-kappa | TTTTGGCCAGGGGACCAAGCTGGAGATCAAACgtgagtcgtacgctagcaagcttgat |
| | | atcgaattctaaactctgagggggtcggatgacgtggccattctttgcctaaagcatt |
| | | gagtttactgcaaggtcagaaaagcatgcaaagccctcagaatggctgcaaagagctc |
| | | caacaaaacaatttagaactttattaaggaatagggggaagctaggaagaaactcaaa |
| | | acatcaagattttaaatacgcttcttggtctccttgctataattatctgggataagca |
| | | tgctgttttctgtctgtccctaacatgccctgtgattatccgcaaacaacacacccaa |
| | | gggcagaactttgttacttaaacaccatcctgtttgcttctttcctcagGAACTGTGG |
| | | CTGCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAAAG |
| | | AGCTTCAACAGGGGAGAGTGT |

1136/1143

| | | |
|---|---|---|
| 92 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1136, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCTACGTTTTCGGTATTGACTATTGGGGCCAGGGAACCCTGGTCACCGTCTCCTCA |
| 93 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1143, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGGCTTACTACGCTGGTCTGTT |
| | VL-kappa | CACTTTTGGCCAGGGGACCAAGCTGGAGATCAAACgtgagtcgtacgctagcaagctt |
| | | gatatcgaattctaaactctgagggggtcggatgacgtggccattctttgcctaaagc |
| | | attgagtttactgcaaggtcagaaaagcatgcaaagccctcagaatggctgcaaagag |
| | | ctccaacaaaacaatttagaactttattaaggaatagggggaagctaggaagaaactc |
| | | aaaacatcaagattttaaatacgcttcttggtctccttgctataattatctgggataa |
| | | gcatgctgttttctgtctgtccctaacatgccctgtgattatccgcaaacaacacacc |
| | | caagggcagaactttgttacttaaacaccatcctgtttgcttctttcctcagGAACTG |
| | | TGGCTGCACCATCTGTCTTCATCTTCCCGCCATCTGATGAGCAGTTGAAATCTGGAAC |
| | | TGCCTCTGTTGTGTGCCTGCTGAATAACTTCTATCCCAGAGAGGCCAAAGTACAGTGG |
| | | AAGGTGGATAACGCCCTCCAATCGGGTAACTCCCAGGAGAGTGTCACAGAGCAGGACA |
| | | GCAAGGACAGCACCTACAGCCTCAGCAGCACCCTGACGCTGAGCAAAGCAGACTACGA |
| | | GAAACACAAAGTCTACGCCTGCGAAGTCACCCATCAGGGCCTGAGCTCGCCCGTCACA |
| | | AAGAGCTTCAACAGGGGAGAGTGT |

1132/1139

| | | |
|---|---|---|
| 94 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1132, | GGCTCCAGGGAAGGGGCTGGAGTGGGTCTCAGGTATTGGTTCTTACGGTGGTGGTACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCTACGTTAACTTCGGTATGGACTATTGGGGCCAGGGAACCCTGGTCACCGTCTCC |
| | | TCA |
| 95 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1139, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGTACGGTCGTAACCCGCCCAC |
| | VL-kappa | TTTTGGCCAGGGGACCAAGCTGGAGATCAAACgtgagtcgtacgctagcaagcttgat |
| | | atcgaattctaaactctgagggggtcggatgacgtggccattctttgcctaaagcatt |
| | | gagtttactgcaaggtcagaaaagcatgcaaagccctcagaatggctgcaaagagctc |
| | | caacaaaacaatttagaactttattaaggaatagggggaagctaggaagaaactcaaa |
| | | acatcaagattttaaatacgcttcttggtctccttgctataattatctgggataagca |
| | | tgctgttttctgtctgtccctaacatgccctgtgattatccgcaaacaacacacccaa |
| | | gggcagaactttgttacttaaacaccatcctgtttgcttctttcctcagGAACTGTGG |
| | | CTGCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAAAG |
| | | AGCTTCAACAGGGGAGAGTGT |

1140/1141

| | | |
|---|---|---|
| 96 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1140, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCGGTCCGGTTTACTCTTCTGTTTTTGACTATTGGGGCCAGGGAACCCTGGTCACC |
| | | GTCTCCTCA |
| 97 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1141, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGAGTTACAGTACCCCTTATAC |
| | VL-kappa | TTTTGGCCAGGGGACCAAGCTGGAGATCAAACgtgagtcgtacgctagcaagcttgat |
| | | atcgaattctaaactctgagggggtcggatgacgtggccattctttgcctaaagcatt |
| | | gagtttactgcaaggtcagaaaagcatgcaaagccctcagaatggctgcaaagagctc |
| | | caacaaaacaatttagaactttattaaggaatagggggaagctaggaagaaactcaaa |
| | | acatcaagattttaaatacgcttcttggtctccttgctataattatctgggataagca |
| | | tgctgttttctgtctgtccctaacatgccctgtgattatccgcaaacaacacacccaa |
| | | gggcagaactttgttacttaaacaccatcctgtttgcttctttcctcagGAACTGTGG |
| | | CTGCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAAAG |
| | | AGCTTCAACAGGGGAGAGTGT |

1150/1152

| | | |
|---|---|---|
| 98 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1150, | GGCTCCAGGGAAGGGGCTGGAGTGGGTCTCAGGTATTGGTGGTTCTTCTTCTTACACA |
| | heavy | TCTTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCTACTACTCTTACCATATGGACTATTGGGGCCAGGGAACCCTGGTCACCGTCTCC |
| | | TCA |
| 99 | Antibody | GACATCCAGATGACCCAGTCTCCATCCTCCCTGAGCGCATCTGTAGGAGACCaCGTCA |
| | clone | CCATCACTTGCCGGGCAAGTCAGAGCATTAGCAGCTATTTAAATTGGTATCAGCAGAA |
| | 1152, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGTACGGTTCTGCTCCGCCCAC |
| | VL-kappa | TTTTGGCCAGGGGACCAAGCTGGAGATCAAACgtgagtcgtacgctagcaagcttgat |
| | | atcgaattctaaactctgagggggtcggatgacgtggccattctttgcctaaagcatt |
| | | gagtttactgcaaggtcagaaaagcatgcaaagccctcagaatggctgcaaagagctc |
| | | caacaaaacaatttagaactttattaaggaatagggggaagctaggaagaaactcaaa |
| | | acatcaagattttaaatacgcttcttggtctccttgctataattatctgggataagca |
| | | tgctgttttctgtctgtccctaacatgccctgtgattatccgcaaacaacacacccaa |
| | | gggcagaactttgttacttaaacaccatcctgtttgcttctttcctcagGAACTGTGG |
| | | CTGCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAAAG |
| | | AGCTTCAACAGGGGAGAGTGT |

1134/1141

| | | |
|---|---|---|
| 100 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1134, | GGCTCCAGGGAAGGGGCTGGAGTGGGTCTCATCTATTTACTCTGGTGGTGGTGGTACA |
| | heavy | TCTTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCGGTCCGGCTTACTCTTCTTTCTTTGACTATTGGGGCCAGGGAACCCTGGTCACC |
| | | GTCTCCTCA |
| 101 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1141, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light, | TGCAACCTGAAGATTTTGCAACTTATTACTGTCAACAGAGTTACAGTACCCCTTATAC |
| | VL-kappa | TTTTGGCCAGGGGACCAAGCTGGAGATCAAACgtgagtcgtacgctagcaagcttgat |
| | | atcgaattctaaactctgagggggtcggatgacgtggccattctttgcctaaagcatt |
| | | gagtttactgcaaggtcagaaaagcatgcaaagccctcagaatggctgcaaagagctc |
| | | caacaaaacaatttagaactttattaaggaatagggggaagctaggaagaaactcaaa |
| | | acatcaagattttaaatacgcttcttggtctccttgctataattatctgggataagca |
| | | tgctgttttctgtctgtccctaacatgccctgtgattatccgcaaacaacacacccaa |
| | | gggcagaactttgttacttaaacaccatcctgtttgcttctttcctcagGAACTGTGG |
| | | CTGCACCATCTGTCTTCATCTTCCCGCCATCTGATGAGCAGTTGAAATCTGGAACTGC |
| | | CTCTGTTGTGTGCCTGCTGAATAACTTCTATCCCAGAGAGGCCAAAGTACAGTGGAAG |
| | | GTGGATAACGCCCTCCAATCGGGTAACTCCCAGGAGAGTGTCACAGAGCAGGACAGCA |
| | | AGGACAGCACCTACAGCCTCAGCAGCACCCTGACGCTGAGCAAAGCAGACTACGAGAA |
| | | ACACAAAGTCTACGCCTGCGAAGTCACCCATCAGGGCCTGAGCTCGCCCGTCACAAAG |
| | | AGCTTCAACAGGGGAGAGTGT |

1146/1147

| | | |
|---|---|---|
| 102 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1146, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCCGTGTTTTCGGTTTTGACTATTGGGGCCAGGGAACCCTGGTCACCGTCTCCTCA |
| 103 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1147, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light VL | TGCAACCTGAAGATTTTGCAACTTATTACTGTCAACAGTACTACTACTACCCGTTCAC |
| | | TTTTGGCCAGGGGACCAAGCTGGAGATCAAA |

1142/1135

| | | |
|---|---|---|
| 104 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1142, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCGGTCCGGCTTACTCTACTGTTTTGGACTATTGGGGCCAGGGAACCCTGGTCACC |
| | | GTCTCCTCA |
| 105 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1135, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light VL | TGCAACCTGAAGATTTTGCAACTTATTACTGTCAACAGAGTTACAGTACCCCTTATAC |
| | | TTTTGGCCAGGGGACCAAGCTGGAGATCAAA |

1148/1149

| | | |
|---|---|---|
| 106 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1148, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCGCTGTTTTCGGTTTTGACTATTGGGGCCAGGGAACCCTGGTCACCGTCTCCTCA |
| 107 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1149, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light VL | TGCAACCTGAAGATTTTGCAACTTATTACTGTCAACAGGCTTACTACTTCCCGCACAC |
| | | TTTTGGCCAGGGGACCAAGCTGGAGATCAAA |

1138/1135

| | | |
|---|---|---|
| 108 | Antibody | GAGGTGCAGCTGTTGGAGAGCGGGGGAGGCTTGGTACAGCCTGGGGGGTCCCTGCGCC |
| | clone | TCTCCTGTGCAGCCAGCGGATTCACCTTTAGCAGCTATGCCATGAGCTGGGTCCGCCA |
| | 1138, | GGCTCCAGGGAAGGGGCTGGAGTGGGTCTCAGCTATTAGTGGTAGTGGTGGTAGCACA |
| | heavy | TACTATGCAGACTCCGTGAAGGGCCGGTTCACCATCTCCCGTGACAATTCCAAGAACA |
| | chain VH | CGCTGTATCTGCAAATGAACAGCCTGCGTGCCGAGGACACGGCTGTATATTATTGTGC |
| | | GCGCGGTTTCGTTTACTCTTCTTACATTGACTATTGGGGCCAGGGAACCCTGGTCACC |
| | | GTCTCCTCA |
| 109 | Antibody | GACATCCAGATGACCCAGTCTCCATCCTCCCTGAGCGCATCTGTAGGAGACCGCGTCA |
| | clone | CCATCACTTGCCGGGCAAGTCAGAGCATTAGCAGCTATTTAAATTGGTATCAGCAGAA |
| | 1135, | ACCAGGGAAAGCCCCTAAGCTCCTGATCTATGCTGCATCCAGTTTGCAAAGTGGGGTC |
| | variable | CCATCACGTTTCAGTGGCAGTGGAAGCGGGACAGATTTCACTCTCACCATCAGCAGTC |
| | light VL | TGCAACCTGAAGATTTTGCAACTTATTACTGTCAACAGAGTTACAGTACCCCTTATAC |
| | | TTTTGGCCAGGGGACCAAGCTGGAGATCAAA |

Representative polynucleotides which encode examples of part B2 may comprise or consist of any one of SEQ ID NOS: 25 to 43 as set out below.

| SEQ | | |
|---|---|---|
| ID | | |
| 25 | 900 | CTCAAAATCCAAGCGTACTTCAACGAAACTGCAGACTTACCGTGTCAGTTTGCCAA |
| | | TTCGCAGAATCAAAGCCTGAGCGAACTGGTGGTTTTCTGGCAGGATCAGGAGAACC |
| | | TGGTTCTGAACGAAGTCTATCTGGGCAAAGAGAAATTCGACAGCGTGGACAGCAAG |
| | | TATATGGGCCGCACCAGCTTTGATAGCGACAGCTGGACCCTGCGTCTGCACAATCT |
| | | GCAAATCAAAGATAAGGGTATCTACCAGTGCGTGATCCACCATAAGAAGCCGAGCG |
| | | GTCTGGTGAAGATTCACGAGATGAACTCCGAGTTGTCTGTCCTGGCG |
| 26 | 901 | CTCAAAATCCAAGCGTACTTCAACGAAACTGCAGACTTACCGTGTCAGTTTGCCAA |
| | | TTCGCAGAATCTGACCCTGAGCGAACTGGTGGTTTTCTGGCAGGATCAGGAGAACC |
| | | TGGTTCTGAACGAAGTCTATCTGGGCAAAGAGAAATTCGACAGCGTGCATAGCAAG |
| | | TATATGGGCCGCACCAGCTTTGATAGCGACAGCTGGACCCTGCGTCTGCACAATCT |
| | | GCAAATCAAAGATAAGGGTATCTACCAGTGCGTGATCCACCATAAGAAGCCGACGG |
| | | GTATGATTAAGATTCACGAGATGAACTCCGAGTTGTCTGTCCTGACC |
| 27 | 904 | CTCAAAATCCAAGCGTACTTCAACGAAACTGCAGACTTACCGTGTCAGTTTGCCAA |
| | | TTCGCAGAATCAAAGCCTGAGCGAACTGATCGTTTTCTGGCAGGATCAGGAGAACC |
| | | TGGTTCTGAACGAAGTCTATCTGGGCAAAGAGCGGTTCGACGCCGTGGACAGCAAG |
| | | TATATGGGCCGCACCAGCTTTGATAGCGACAGCTGGACCCTGCGTCTGCACAATCT |
| | | GCAAATCAAAGATAAGGGTATCTACCAGTGCATTATCCACCATAAGAAGCCGAGCG |
| | | GTATGGTGAAGATTCACCAAATGGACTCCGAGTTGTCTGTCCTGGCG |
| 28 | 906 | CTCAAAATCCAAGCGTACATCAACGAAACTGCAGACTTACCGTGTCAGTTTGCCAA |
| | | TTCGCAGAATCTGAGCCTGAGCGAACTGGTGGTTTTCTGGCAGGATCAGGAGAACC |
| | | TGGTTCTGAACGAAGTCTATCTGGGCAAAGAGCGGTTCGACAGCGTGGACAGCAAG |
| | | TATATGGGCCGCACCAGCTTTGATAGCGACAGCTGGACCCTGCGTCTGCACAATCT |
| | | GCAAATCAAAGATAAGGGTTTCTACCAGTGCATTATCCACCATAAGAAGCCGACGG |
| | | GTCTGGTGAAGATTCACGAGATGAACTCCGAGTTGTCTGTCCTGGCG |
| 29 | 907 | CTCAAAATCCAAGCGTACTTCAACGAAACTGCAGACTTACCGTGTCAGTTTGCCAA |
| | | TTCGCAGAATCAAAGCCTGAGCGAACTGGTGGTTTTCTGGCAGGATCAGGAGAACC |
| | | TGGTTCTGAACGAAGTCTATCTGGGCAAAGAGAAATTCGACAGCGTGCATAGCAAG |
| | | TATATGGGCCGCACCAGCTTTGATAGCGACAGCTGGACCCTGCGTCTGCACAATCT |
| | | GCAAATCAAAGATAAGGGTCTGTACCAGTGCATTATCCACCATAAGAAGCCGACGG |
| | | GTATGATTAAGATTCACGAGATGAACTCCGAGTTGTCTGTCCTGGCG |
| 30 | 908 | CTCAAAATCCAAGCGTACTTCAACGAAACTGCAGACTTACCGTGTCAGTTTGCCAA |
| | | TTCGCAGAATCAAAGCCTGAGCGAACTGGTGGTTTTCTGGCAGGATCAGGAGAACC |
| | | TGGTTCTGAACGAAGTCTATCTGGGCAAAGAGAAATTCGACAGCGTGCATAGCAAG |
| | | TATATGGGCCGCACCAGCTTTGATAGCGACAGCTGGACCCTGCGTCTGCACAATCT |
| | | GCAAATCAAAGATAAGGGTATCTACCAGTGCATTATCCACCATAAGAAGCCGACGG |
| | | GTATGGTGAAGATTCACGAGATGAACTCCGAGTTGTCTGTCCTGGCG |
| 31 | 910 | CTCAAAATCCAAGCGTACTTCAACGAAACTGCAGACTTACCGTGTCAGTTTGCCAA |
| | | TTCGCAGAATCAAAGCCTGAGCGAACTGGTGGTTTTCTGGCAGGATCAGGAGAACC |
| | | TGGTTCTGAACGAAGTCTATCTGGGCAAAGAGAAATTCGACAGCGTGGACAGCAAG |
| | | TATATGGGCCGCACCAGCTTTGATAGCGACAGCTGGACCCTGCGTCTGCACAATCT |
| | | GCAAATCAAAGATAAGGGTATCTACCAGTGCATTATCCACCATAAGAAGCCGACGG |
| | | GTATGGTGAAGATTCACGAGATGAACTCCGAGTTGTCTGTCCTGGCG |
| 32 | 915 | CTCAAAATCCAAGCGTACTTCAACGAAACTGCAGACTTACCGTGTCAGTTTGCCAA |
| | | TTCGCAGAATCAAAGCCTGAGCGAACTGGTGGTTTTCTGGCAGGATCAGGAGAACC |
| | | TGATCCTGAACGAAGTCTATCTGGGCAAAGAGAAATTCGACAGCGTGGACAGCAAG |
| | | TATATGGGCCGCACCAGCTTTGATAGCGACAGCTGGACCCTGCGTCTGCACAATCT |
| | | GCAAATCAAAGATAAGGGTTTCTACCAGTGCATTATCCACCATAAGAAGCCGAGCG |
| | | GTCTGATTAAGATTCACCAAATGGACTCCGAGTTGTCTGTCCTGGCG |
| 33 | 938 | CTCAAAATCCAAGCGTACTTCAACGAAACTGCAGACTTACCGTGTCAGTTTGCCAA |
| | | TTCGCAGAATCTGAGCCTGAGCGAACTGGTGGTTTTCTGGCAGGATCAGGAGAACC |
| | | TGATCCTGAACGAAGTCTATCTGGGCAAAGAGCGGTTCGACAGCGTGCATAGCAAG |
| | | TATATGGGCCGCACCAGCTTTGATAGCGACAGCTGGACCCTGCGTCTGCACAATCT |
| | | GCAAATCAAAGATAAGGGTCTGTACCAGTGCATTATCCACCATAAGAAGCCGAGCG |
| | | GTATGGTGAAGATTCACGAGATGAACTCCGAGTTGTCTGTCCTGGCG |
| 34 | 1038 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGAAATTCGACAGCGTGGAC |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGACGGGTATGGTGAAGATTCACGAGATGAACTCCGAGTTGTCTGTCCTGGCG |
| 35 | 1039 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGAAATTCGACAGCGTGAGT |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGAGCGGTATGGTGAAGATTCACCAAATGGACTCCGAGTTGTCTGTCCTGGCG |
| 36 | 1040 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGCGGTTCGACAGCGTGGAC |
| | | AGCAAGTATATGGGCCGCACCAGCTTTGATAGCGACAGCTGGACCCTGCGTCTGCA |
| | | CAATCTGCAAATCAAAGATAAGGGTAGGTACCAGTGCATTATCCACCATAAGAAGC |
| | | CGACGGGTATGATTAATATTCACCAAATGAACTCCGAGTTGTCTGTCCTGGCG |
| 37 | 1041 | GCCCCCCTCAAAATCCAAGCGTACCTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGAAATTCGACAGCGTGGAC |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGACGGGTCTGGTGAAGATTCACGAGATGAACTCCGAGTTGTCTGTCCTGGCG |
| 38 | 1042 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGATTTTCGACAGCGTGAGT |
| | | AGCAAGTATATGGGCCGCACCAGCTTTGATAGTGACAGCTGGACCCTGCGTCTGCA |
| | | CAATCTGCAAATCAAAGATAAGGGTATCTACCAGTGCATTATCCACCATAAGAAGC |
| | | CGAGCGGTATGGTGAAGATTCACCAAATGGACTCCGAGTTGTCTGTCCTGGCG |
| 39 | 1043 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGAAATTCGACAGCGTGGAT |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGACGGGTATGATTAAGATTCACGAGATGAACTCCGAGTTGTCTGTCCTGGCG |
| 40 | 1044 | GCCCCCCTCAAAATCCAAGCGTACTTCAACGAAACTGCAGACTTACCGTGTCAGTT |
| | | TGCCAATTCGCAGAATCTGACCCTGAGCGAACTGGTGGTTTTCTGGCAGGATCAGG |
| | | AGAACCTGGTTCTGAACGAAGTCTATCTGGGCAAAGAGAAATTCGACAGCGTGTCT |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGACGGGTATGATTAAGATTCACGAGATGAGCTCCGAGTTGTCTGTCCTGGCG |
| 41 | 1045 | GCCCCCCTCAAAATCCAAGCGTACTTCAACGAAACTGCAGACTTACCGTGTCAGTT |
| | | TGCCAATTCGCAGAATCTGACCCTGAGCGAACTGGTGGTTTTCTGGCAGGATCAGG |
| | | AGAACCTGGTTCTGAACGAAGTCTATCTGGGCAAAGAGAAATTCGACAGCGTGGAC |
| | | AGCAAGTATATGGGCCGCACCAGCTTTGATAGCGACAGCTGGACCCTGCGTCTGCA |
| | | CAATCTGCAAATCAAAGATAAGGGTCTGTACCAGTGCATTATCCACCATAAGAAGC |
| | | CGACGGGTCTGGTGAAGATTCACGAGATGAACTCCGAGTTGTCTGTCCTGGCG |
| 42 | 1046 | GCCCCCCTCAAAATCCAAGCGTACTTCAACGAAACTGCAGACTTACCGTGTCAGTT |
| | | TGCCAATTCGCAGAATCAAAGCCTGAGCGAACTGGTGGTTTTCTGGCAGGATCAGG |
| | | AGAACCTGGTTCTGAACGAAGTCTATCTGGGCAAAGAGAAATTCGACAGCGTGGAC |
| | | AGCAAGTATATGGGCCGCACCAGCTTTGATAGCGACAGCTGGACCCTGCGTCTGCA |
| | | CAATCTGCAAATCGAAGATAAGGGTATCTACCAGTGCATTATCCACCATAAGAAGC |
| | | CGAGCGGTATGGTGAAGATTCACCAAATGGACTCCGAGTTGTCTGTCCTGGCG |
| 43 | 1047 | GCCCCCCTCAAAATCCAAGCGTACTTCAACGAAACTGCAGACTTACCGTGTCAGTT |
| | | TGCCAATTCGCAGAATCTGAGCCTGAGCGAACTGGTGGTTTTCTGGCAGGATCAGG |
| | | AGAACCTGGTTCTGAACGAAGTCTATCTGGGCAAAGAGAAATTCGACAGCGTGGAC |
| | | AGCAAGTATATGGGCCGCACCAGCTTTGATAGCGACAGCTGGACCCTGCGTCTGCA |
| | | CAATCTGCAAATCAAAGATAAGGGTATCTACCAGTGCATTATCCACCATAAGAAGC |
| | | CGACGGGTCTGGTGAAGATTCACGAGATGAACTCCGAGTTGTCTGTCCTGGCG |

Representative polynucleotides which encode the polypeptides of SEQ ID NOs: 56 to 50 and 110 to 114 may comprise or consist of any one of SEQ ID NOS: 65 to 69 and 115 to 119 as set out below.

| | | |
|---|---|---|
| 65 | Bispecific | CAGTCCGTGCTGACCCAGCCACCCTCCGCCAGCGGCACCCCTGGCCAGCGGGT |
| | molecule | GACCATCTCTTGTAGCGGCAGCAGCTCCAACATCGGAAGCAATACCGTGAATT |
| | 956/530: | GGTACCAGCAGCTGCCCGGCACCGCCCCCAAGCTGCTGATCTACCGCAATAAC |
| | A2-54 | CAGAGGCCCTCCGGCGTGCCCGACAGGTTCAGCGGCAGCAAGTCTGGCACCTC |
| | light chain, | CGCCTCTCTGGCCATTAGCGGACTGCGGAGCGAGGACGAGGCCGATTACTACT |
| | VL-kappa | GCGCCGCCTGGGACGACTCCCTGTCCGGGTGGCGCTTTGGAGGCGGCACAAAG |
| | linker: | CTGACCGTGCTGGGAGGTGAGTAGAACGTACGCTAGCAAGCTTGATATCGAAT |
| | SGGGGSGGGGS | TCTAAACTCTGAGGGGGTCGGATGACGTGGCCATTCTTTGCCTAAAGCATTGA |
| | 904, | GTTTACTGCAAGGTCAGAAAAGCATGCAAAGCCCTCAGAATGGCTGCAAAGAG |
| | CD86 mutant | CTCCAACAAAACAATTTAGAACTTTATTAAGGAATAGGGGGAAGCTAGGAAGA |
| | molecule | AACTCAAAACATCAAGATTTTAAATACGCTTCTTGGTCTCCTTGCTATAATTA |
| | | TCTGGGATAAGCATGCTGTTTTCTGTCTGTCCCTAACATGCCCTGTGATTATC |
| | | CGCAAACAACACACCCAAGGGCAGAACTTTGTTACTTAAACACCATCCTGTTT |
| | | GCTTCTTTCCTCAGGAACTGTGGCTGCACCATCTGTCTTCATCTTCCCGCCAT |
| | | CTGATGAGCAGTTGAAATCTGGAACTGCCTCTGTTGTGTGCCTGCTGAATAAC |
| | | TTCTATCCCAGAGAGGCCAAAGTACAGTGGAAGGTGGATAACGCCCTCCAATC |
| | | GGGTAACTCCCAGGAGAGTGTCACAGAGCAGGACAGCAAGGACAGCACCTACA |
| | | GCCTCAGCAGCACCCTGACGCTGAGCAAAGCAGACTACGAGAAACACAAAGTC |
| | | TACGCCTGCGAAGTCACCCATCAGGGCCTGAGCTCGCCCGTCACAAAGAGCTT |
| | | CAACAGGGGAGAGTGTAGCGGAGGAGGAGGAAGCGGAGGAGGAGGAAGCCTCA |
| | | AGATTCAGGCGTACTTTAACGAAACAGCGGACCTCCCGTGCCAATTCGCCAAT |
| | | AGCCAGAATCAATCGCTCAGTGAGCTCATCGTGTTTTGGCAAGATCAGGAAAA |
| | | CCTTGTCTTGAATGAGGTCTATCTGGGTAAAGAGAGATTCGACGCCGTAGATT |
| | | CCAAGTACATGGGGAGGACTTCGTTCGATTCAGATTCGTGGACGCTGAGATTG |
| | | CATAATCTCCAAATCAAAGACAAAGGGATCTACCAGTGCATCATTCACCACAA |
| | | AAAGCCTAGCGGAATGGTGAAGATCCATCAGATGGACAGCGAATTGTCAGTTT |
| | | TGGCC |
| 66 | Bispecific | CAGTCCGTGCTGACCCAGCCACCCTCCGCCAGCGGCACCCCTGGCCAGCGGGT |
| | Molecule | GACCATCTCTTGTAGCGGCAGCAGCTCCAACATCGGAAGCAATACCGTGAATT |
| | 957/530: | GGTACCAGCAGCTGCCCGGCACCGCCCCCAAGCTGCTGATCTACCGCAATAAC |
| | A2-54 | CAGAGGCCCTCCGGCGTGCCCGACAGGTTCAGCGGCAGCAAGTCTGGCACCTC |
| | light chain, | CGCCTCTCTGGCCATTAGCGGACTGCGGAGCGAGGACGAGGCCGATTACTACT |
| | VL-kappa | GCGCCGCCTGGGACGACTCCCTGTCCGGGTGGCGCTTTGGAGGCGGCACAAAG |
| | linker: | CTGACCGTGCTGGGAGGTGAGTAGAACGTACGCTAGCAAGCTTGATATCGAAT |
| | SGGGGSGGGGS | TCTAAACTCTGAGGGGGTCGGATGACGTGGCCATTCTTTGCCTAAAGCATTGA |
| | Amino acids | GTTTACTGCAAGGTCAGAAAAGCATGCAAAGCCCTCAGAATGGCTGCAAAGAG |
| | inserted AP. | CTCCAACAAAACAATTTAGAACTTTATTAAGGAATAGGGGGAAGCTAGGAAGA |
| | 904, | AACTCAAAACATCAAGATTTTAAATACGCTTCTTGGTCTCCTTGCTATAATTA |
| | mutant CD86 | TCTGGGATAAGCATGCTGTTTTCTGTCTGTCCCTAACATGCCCTGTGATTATC |
| | molecule | CGCAAACAACACACCCAAGGGCAGAACTTTGTTACTTAAACACCATCCTGTTT |
| | | GCTTCTTTCCTCAGGAACTGTGGCTGCACCATCTGTCTTCATCTTCCCGCCAT |
| | | CTGATGAGCAGTTGAAATCTGGAACTGCCTCTGTTGTGTGCCTGCTGAATAAC |
| | | TTCTATCCCAGAGAGGCCAAAGTACAGTGGAAGGTGGATAACGCCCTCCAATC |
| | | GGGTAACTCCCAGGAGAGTGTCACAGAGCAGGACAGCAAGGACAGCACCTACA |
| | | GCCTCAGCAGCACCCTGACGCTGAGCAAAGCAGACTACGAGAAACACAAAGTC |
| | | TACGCCTGCGAAGTCACCCATCAGGGCCTGAGCTCGCCCGTCACAAAGAGCTT |
| | | CAACAGGGGAGAGTGTAGCGGAGGAGGAGGAAGCGGAGGAGGAGGAAGCGCCC |
| | | CCCTCAAGATTCAGGCGTACTTTAACGAAACAGCGGACCTCCCGTGCCAATTC |
| | | GCCAATAGCCAGAATCAATCGCTCAGTGAGCTCATCGTGTTTTGGCAAGATCA |
| | | GGAAAACCTTGTCTTGAATGAGGTCTATCTGGGTAAAGAGAGATTCGACGCCG |
| | | TAGATTCCAAGTACATGGGGAGGACTTCGTTCGATTCAGATTCGTGGACGCTG |
| | | AGATTGCATAATCTCCAAATCAAAGACAAAGGGATCTACCAGTGCATCATTCA |
| | | CCACAAAAAGCCTAGCGGAATGGTGAAGATCCATCAGATGGACAGCGAATTGT |
| | | CAGTTTTGGCC |
| 67 | Bispecific | CTCAAGATTCAGGCGTACTTTAACGAAACAGCGGACCTCCCGTGCCAATTCGC |
| | molecule | CAATAGCCAGAATCAATCGCTCAGTGAGCTCATCGTGTTTTGGCAAGATCAGG |
| | 958/531: | AAAACCTTGTCTTGAATGAGGTCTATCTGGGTAAAGAGAGATTCGACGCCGTA |
| | 904, | GATTCCAAGTACATGGGGAGGACTTCGTTCGATTCAGATTCGTGGACGCTGAG |
| | mutant CD86 | ATTGCATAATCTCCAAATCAAAGACAAAGGGATCTACCAGTGCATCATTCACC |
| | molecule | ACAAAAAGCCTAGCGGAATGGTGAAGATCCATCAGATGGACAGCGAATTGTCA |
| | linker: | GTTTTGGCCAACTTCAGCCAGCCCGAGGTGCAGCTGCTGGAGTCCGGAGGAGG |
| | NFSQP | CCTGGTGCAGCCCGGCGGCAGCCTGAGACTGAGCTGTGCCGCCAGCGGATTCA |
| | A2-54 | CCTTCTCCGACTACTATATGAGCTGGGTGAGGCAGGCCCCAGGCAAGGGCCTG |
| | Heavy | GAGTGGATTTCCTCCATCAGCAGCAATGGGATCTACATTTACTACGCCGACAG |
| | Chain, | CCTGAAGGGCAGGTTCACAATCAGCAGGGACAACTCTAAGAACACACTGTACC |
| | VH-gamma1 | TGCAGATGAACTCCCTGCGCGCCGAGGACACCGCCGTGTATTACTGTGCCAGG |
| | | GCCCCCGTGGACTACTCTAATCCCAGCGGCATGGACGTGTGGGGCCAGGGCAC |
| | | CCTGGTGACAGTGAGCTCAGGTGAGTCGTACGCTAGCAAGCTTTCTGGGGCAG |
| | | GCCAGGCCTGACCTTGGCTTTGGGGCAGGGAGGGGGCTAAGGTGAGGCAGGTG |
| | | GCGCCAGCCAGGTGCACACCCAATGCCCATGAGCCCAGACACTGGACGCTGAA |
| | | CCTCGCGGACAGTTAAGAACCCAGGGGCCTCTGCGCCCTGGGCCCAGCTCTGT |
| | | CCCACACCGCGGTCACATGGCACCACCTCTCTTGCAGCCTCCACCAAGGGCCC |
| | | ATCGGTCTTCCCCCTGGCACCCTCCTCCAAGAGCACCTCTGGGGGCACAGCGG |
| | | CCCTGGGCTGCCTGGTCAAGGACTACTTCCCCGAACCGGTGACGGTGTCGTGG |
| | | AACTCAGGCGCCCTGACCAGCGGCGTGCACACCTTCCCGGCTGTCCTACAGTC |
| | | CTCAGGACTCTACTCCCTCAGCAGCGTGGTGACCGTGCCCTCCAGCAGCTTGG |
| | | GCACCCAGACCTACATCTGCAACGTGAATCACAAGCCCAGCAACACCAAGGTG |
| | | GACAAGAAAGTTGGTGAGAGGCCAGCACAGGGAGGGAGGGTGTCTGCTGGAAG |
| | | CCAGGCTCAGCGCTCCTGCCTGGACGCATCCCGGCTATGCAGCCCCAGTCCAG |
| | | GGCAGCAAGGCAGGCCCCGTCTGCCTCTTCACCCGGAGGCCTCTGCCCGCCCC |
| | | ACTCATGCTCAGGGAGAGGGTCTTCTGGCTTTTTCCCCAGGCTCTGGGCAGGC |
| | | ACAGGCTAGGTGCCCCTAACCCAGGCCCTGCACACAAAGGGGCAGGTGCTGGG |
| | | CTCAGACCTGCCAAGAGCCATATCCGGGAGGACCCTGCCCCTGACCTAAGCCC |
| | | ACCCCAAAGGCCAAACTCTCCACTCCCTCAGCTCGGACACCTTCTCTCCTCCC |
| | | AGATTCCAGTAACTCCCAATCTTCTCTCTGCAGAGCCCAAATCTTGTGACAAA |
| | | ACTCACACATGCCCACCGTGCCCAGGTAAGCCAGCCCAGGCCTCGCCCTCCAG |
| | | CTCAAGGCGGGACAGGTGCCCTAGAGTAGCCTGCATCCAGGGACAGGCCCCAG |
| | | CCGGGTGCTGACACGTCCACCTCCATCTCTTCCTCAGCACCTGAACTCCTGGG |
| | | GGGACCGTCAGTCTTCCTCTTCCCCCCAAAACCCAAGGACACCCTCATGATCT |
| | | CCCGGACCCCTGAGGTCACATGCGTGGTGGTGGACGTGAGCCACGAAGACCCT |
| | | GAGGTCAAGTTCAACTGGTACGTGGACGGCGTGGAGGTGCATAATGCCAAGAC |
| | | AAAGCCGCGGGAGGAGCAGTACAACAGCACGTACCGTGTGGTCAGCGTCCTCA |
| | | CCGTCCTGCACCAGGACTGGCTGAATGGCAAGGAGTACAAGTGCAAGGTCTCC |
| | | AACAAAGCCCTCCCAGCCCCCATCGAGAAAACCATCTCCAAAGCCAAAGGTGG |
| | | GACCCGTGGGGTGCGAGGGCCACATGGACAGAGGCCGGCTCGGCCCACCCTCT |
| | | GCCCTGAGAGTGACCGCTGTACCAACCTCTGTCCCTACAGGGCAGCCCCGAGA |
| | | ACCACAGGTGTACACCCTGCCCCCATCCCGGGATGAGCTGACCAAGAACCAGG |
| | | TCAGCCTGACCTGCCTGGTCAAAGGCTTCTATCCCAGCGACATCGCCGTGGAG |
| | | TGGGAGAGCAATGGGCAGCCGGAGAACAACTACAAGACCACGCCTCCCGTGCT |
| | | GGACTCCGACGGCTCCTTCTTCCTCTACAGCAAGCTCACCGTGGACAAGAGCA |
| | | GGTGGCAGCAGGGGAACGTCTTCTCATGCTCCGTGATGCATGAGGCTCTGCAC |
| | | AACCACTACACGCAGAAGAGCCTCTCCCTGTCTCCGGGTAAATGAG |
| 68 | Bispecific | CTCAAGATTCAGGCGTACTTTAACGAAACAGCGGACCTCCCGTGCCAATTCGC |
| | molecule | CAATAGCCAGAATCAATCGCTCAGTGAGCTCATCGTGTTTTGGCAAGATCAGG |
| | 959/530: | AAAACCTTGTCTTGAATGAGGTCTATCTGGGTAAAGAGAGATTCGACGCCGTA |
| | 904, | GATTCCAAGTACATGGGGAGGACTTCGTTCGATTCAGATTCGTGGACGCTGAG |
| | mutant CD86 | ATTGCATAATCTCCAAATCAAAGACAAAGGGATCTACCAGTGCATCATTCACC |
| | molecule | ACAAAAAGCCTAGCGGAATGGTGAAGATCCATCAGATGGACAGCGAATTGTCA |
| | linker: | GTTTTGGCCAAGAGAACCGTGGCCCAGTCCGTGCTGACCCAGCCACCCTCCGC |
| | KRTVA | CAGCGGCACCCCTGGCCAGCGGGTGACCATCTCTTGTAGCGGCAGCAGCTCCA |
| | A2-54 | ACATCGGAAGCAATACCGTGAATTGGTACCAGCAGCTGCCCGGCACCGCCCCC |
| | Variable | AAGCTGCTGATCTACCGCAATAACCAGAGGCCCTCCGGCGTGCCCGACAGGTT |
| | Light chain, | CAGCGGCAGCAAGTCTGGCACCTCCGCCTCTCTGGCCATTAGCGGACTGCGGA |
| | VL-kappa | GCGAGGACGAGGCCGATTACTACTGCGCCGCCTGGGACGACTCCCTGTCCGGG |
| | | TGGCGCTTTGGAGGCGGCACAAAGCTGACCGTGCTGGGAGGTGAGTAGAACGT |
| | | ACGCTAGCAAGCTTGATATCGAATTCTAAACTCTGAGGGGGTCGGATGACGTG |
| | | GCCATTCTTTGCCTAAAGCATTGAGTTTACTGCAAGGTCAGAAAAGCATGCAA |
| | | AGCCCTCAGAATGGCTGCAAAGAGCTCCAACAAAACAATTTAGAACTTTATTA |
| | | AGGAATAGGGGGAAGCTAGGAAGAAACTCAAAACATCAAGATTTTAAATACGC |
| | | TTCTTGGTCTCCTTGCTATAATTATCTGGGATAAGCATGCTGTTTTCTGTCTG |
| | | TCCCTAACATGCCCTGTGATTATCCGCAAACAACACACCCAAGGGCAGAACTT |
| | | TGTTACTTAAACACCATCCTGTTTGCTTCTTTCCTCAGGAACTGTGGCTGCAC |
| | | CATCTGTCTTCATCTTCCCGCCATCTGATGAGCAGTTGAAATCTGGAACTGCC |
| | | TCTGTTGTGTGCCTGCTGAATAACTTCTATCCCAGAGAGGCCAAAGTACAGTG |
| | | GAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCT |
| | | GAGCTCGCCCGTCACAAAGAGCTTCAACAGGGGAGAGTGT |
| 69 | Bispecific | CTCAAGATTCAGGCGTACTTTAACGAAACAGCGGACCTCCCGTGCCAATTCGC |
| | molecule | CAATAGCCAGAATCAATCGCTCAGTGAGCTCATCGTGTTTTGGCAAGATCAGG |
| | 960/530: | AAAACCTTGTCTTGAATGAGGTCTATCTGGGTAAAGAGAGATTCGACGCCGTA |
| | 904, | GATTCCAAGTACATGGGGAGGACTTCGTTCGATTCAGATTCGTGGACGCTGAG |
| | mutant CD86 | ATTGCATAATCTCCAAATCAAAGACAAAGGGATCTACCAGTGCATCATTCACC |
| | molecule | ACAAAAAGCCTAGCGGAATGGTGAAGATCCATCAGATGGACAGCGAATTGTCA |
| | linker: | GTTTTGGCCAACTTCAGCCAGCCCCAGTCCGTGCTGACCCAGCCACCCTCCGC |
| | NFSQP | CAGCGGCACCCCTGGCCAGCGGGTGACCATCTCTTGTAGCGGCAGCAGCTCCA |
| | A2-54 | ACATCGGAAGCAATACCGTGAATTGGTACCAGCAGCTGCCCGGCACCGCCCCC |
| | Variable | AAGCTGCTGATCTACCGCAATAACCAGAGGCCCTCCGGCGTGCCCGACAGGTT |
| | Light, | CAGCGGCAGCAAGTCTGGCACCTCCGCCTCTCTGGCCATTAGCGGACTGCGGA |
| | VL-kappa | GCGAGGACGAGGCCGATTACTACTGCGCCGCCTGGGACGACTCCCTGTCCGGG |
| | | TGGCGCTTTGGAGGCGGCACAAAGCTGACCGTGCTGGGAGGTGAGTAGAACGT |
| | | ACGCTAGCAAGCTTGATATCGAATTCTAAACTCTGAGGGGGTCGGATGACGTG |
| | | GCCATTCTTTGCCTAAAGCATTGAGTTTACTGCAAGGTCAGAAAAGCATGCAA |
| | | AGCCCTCAGAATGGCTGCAAAGAGCTCCAACAAAACAATTTAGAACTTTATTA |
| | | AGGAATAGGGGGAAGCTAGGAAGAAACTCAAAACATCAAGATTTTAAATACGC |
| | | TTCTTGGTCTCCTTGCTATAATTATCTGGGATAAGCATGCTGTTTTCTGTCTG |
| | | TCCCTAACATGCCCTGTGATTATCCGCAAACAACACACCCAAGGGCAGAACTT |
| | | TGTTACTTAAACACCATCCTGTTTGCTTCTTTCCTCAGGAACTGTGGCTGCAC |
| | | CATCTGTCTTCATCTTCCCGCCATCTGATGAGCAGTTGAAATCTGGAACTGCC |
| | | TCTGTTGTGTGCCTGCTGAATAACTTCTATCCCAGAGAGGCCAAAGTACAGTG |
| | | GAAGGTGGATAACGCCCTCCAATCGGGTAACTCCCAGGAGAGTGTCACAGAGC |
| | | AGGACAGCAAGGACAGCACCTACAGCCTCAGCAGCACCCTGACGCTGAGCAAA |
| | | GCAGACTACGAGAAACACAAAGTCTACGCCTGCGAAGTCACCCATCAGGGCCT |
| | | GAGCTCGCCCGTCACAAAGAGCTTCAACAGGGGAGAGTGT |
| 115 | Bispecific | GACATCCAGATGACCCAGTCTCCATCCTCCCTGAGCGCATCTGTAGGAGACCG |
| | molecule | CGTCACCATCACTTGCCGGGCAAGTCAGAGCATTAGCAGCTATTTAAATTGGT |
| | 1107/1145: | ATCAGCAGAAACCAGGGAAAGCCCCTAAGCTCCTGATCTATGCTGCATCCAGT |
| | clone 1145 | TTGCAAAGTGGGGTCCCATCACGTTTCAGTGGCAGTGGAAGCGGGACAGATTT |
| | light | CACTCTCACCATCAGCAGTCTGCAACCTGAAGATTTTGCAACTTATTACTGTC |
| | chain, | AACAGTACGGTGTTTACCCGTTCACTTTTGGCCAGGGGACCAAGCTGGAGATC |
| | VL-kappa, | AAACgtgagtcgtacgctagcaagcttgatatcgaattctaaactctgagggg |
| | Linker | gtcggatgacgtggccattctttgcctaaagcattgagtttactgcaaggtca |
| | SGGGGSGGGGS, | gaaaagcatgcaaagccctcagaatggctgcaaagagctccaacaaaacaatt |
| | 1040: CD86 | tagaactttattaaggaatagggggaagctaggaagaaactcaaaacatcaag |
| | mutant | attttaaatacgcttcttggtctccttgctataattatctgggataagcatgc |
| | molecule | tgttttctgtctgtccctaacatgccctgtgattatccgcaaacaacacaccc |
| | | aagggcagaactttgttacttaaacaccatcctgtttgcttctttcctcagGA |
| | | ACTGTGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAGTCA |
| | | CCCATCAGGGCCTGAGCTCGCCCGTCACAAAGAGCTTCAACAGGGGAGAGTGT |
| | | AGCGGAGGAGGAGGAAGCGGAGGAGGAGGAAGCGCCCCCCTCAAAATCCAAGC |
| | | GTACTTCAACGAAACTGCAGACTTACCGTGTCAGTTTGCCAATTCGCAGAATC |
| | | TGAGCCTGAGCGAACTGGTGGTTTTCTGGCAGGATCAGGAGAACCTGGTTCTG |
| | | AACGAAGTCTATCTGGGCAAAGAGCGGTTCGACAGCGTGGACAGCAAGTATAT |
| | | GGGCCGCACCAGCTTTGATAGCGACAGCTGGACCCTGCGTCTGCACAATCTGC |
| | | AAATCAAAGATAAGGGTAGGTACCAGTGCATTATCCACCATAAGAAGCCGACG |
| | | GGTATGATTAATATTCACCAAATGAACTCCGAGTTGTCTGTCCTGGCG |
| 116 | Bispecific | GACATCCAGATGACCCAGTCTCCATCCTCCCTGAGCGCATCTGTAGGAGACCG |
| | molecule | CGTCACCATCACTTGCCGGGCAAGTCAGAGCATTAGCAGCTATTTAAATTGGT |
| | 1136/1143: | ATCAGCAGAAACCAGGGAAAGCCCCTAAGCTCCTGATCTATGCTGCATCCAGT |
| | clone 1143 | TTGCAAAGTGGGGTCCCATCACGTTTCAGTGGCAGTGGAAGCGGGACAGATTT |
| | light | CACTCTCACCATCAGCAGTCTGCAACCTGAAGATTTTGCAACTTATTACTGTC |
| | chain, | AACAGGCTTACTACGCTGGTCTGTTCACTTTTGGCCAGGGGACCAAGCTGGAG |
| | VL-kappa, | ATCAAACgtgagtcgtacgctagcaagcttgatatcgaattctaaactctgag |
| | Linker | ggggtcggatgacgtggccattctttgcctaaagcattgagtttactgcaagg |
| | SGGGGSGGGGS, | tcagaaaagcatgcaaagccctcagaatggctgcaaagagctccaacaaaaca |
| | 1040: CD86 | atttagaactttattaaggaatagggggaagctaggaagaaactcaaaacatc |
| | mutant | aagattttaaatacgcttcttggtctccttgctataattatctgggataagca |
| | molecule | tgctgttttctgtctgtccctaacatgccctgtgattatccgcaaacaacaca |
| | | cccaagggcagaactttgttacttaaacaccatcctgtttgcttctttcctca |
| | | gGAACTGTGGCTGCACCATCTGTCTTCATCTTCCCGCCATCTGATGAGCAGTT |
| | | GAAATCTGGAACTGCCTCTGTTGTGTGCCTGCTGAATAACTTCTATCCCAGAG |
| | | AGGCCAAAGTACAGTGGAAGGTGGATAACGCCCTCCAATCGGGTAACTCCCAG |
| | | GAGAGTGTCACAGAGCAGGACAGCAAGGACAGCACCTACAGCCTCAGCAGCAC |
| | | CCTGACGCTGAGCAAAGCAGACTACGAGAAACACAAAGTCTACGCCTGCGAAG |
| | | TCACCCATCAGGGCCTGAGCTCGCCCGTCACAAAGAGCTTCAACAGGGGAGAG |
| | | TGTAGCGGAGGAGGAGGAAGCGGAGGAGGAGGAAGCGCCCCCCTCAAAATCCA |
| | | AGCGTACTTCAACGAAACTGCAGACTTACCGTGTCAGTTTGCCAATTCGCAGA |
| | | ATCTGAGCCTGAGCGAACTGGTGGTTTTCTGGCAGGATCAGGAGAACCTGGTT |
| | | CTGAACGAAGTCTATCTGGGCAAAGAGCGGTTCGACAGCGTGGACAGCAAGTA |
| | | TATGGGCCGCACCAGCTTTGATAGCGACAGCTGGACCCTGCGTCTGCACAATC |
| | | TGCAAATCAAAGATAAGGGTAGGTACCAGTGCATTATCCACCATAAGAAGCCG |
| | | ACGGGTATGATTAATATTCACCAAATGAACTCCGAGTTGTCTGTCCTGGCG |
| 117 | Bispecific | GACATCCAGATGACCCAGTCTCCATCCTCCCTGAGCGCATCTGTAGGAGACCG |
| | molecule | CGTCACCATCACTTGCCGGGCAAGTCAGAGCATTAGCAGCTATTTAAATTGGT |
| | 1132/1139: | ATCAGCAGAAACCAGGGAAAGCCCCTAAGCTCCTGATCTATGCTGCATCCAGT |
| | clone 1139 | TTGCAAAGTGGGGTCCCATCACGTTTCAGTGGCAGTGGAAGCGGGACAGATTT |
| | light | CACTCTCACCATCAGCAGTCTGCAACCTGAAGATTTTGCAACTTATTACTGTC |
| | chain, | AACAGTACGGTCGTAACCCGCCCACTTTTGGCCAGGGGACCAAGCTGGAGATC |
| | VL-kappa, | AAACgtgagtcgtacgctagcaagcttgatatcgaattctaaactctgagggg |
| | Linker | gtcggatgacgtggccattctttgcctaaagcattgagtttactgcaaggtca |
| | SGGGGSGGGGS, | gaaaagcatgcaaagccctcagaatggctgcaaagagctccaacaaaacaatt |
| | 1040: CD86 | tagaactttattaaggaatagggggaagctaggaagaaactcaaaacatcaag |
| | mutant | attttaaatacgcttcttggtctccttgctataattatctgggataagcatgc |
| | molecule | tgttttctgtctgtccctaacatgccctgtgattatccgcaaacaacacaccc |
| | | aagggcagaactttgttacttaaacaccatcctgtttgcttctttcctcagGA |
| | | ACTGTGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAGTCA |
| | | CCCATCAGGGCCTGAGCTCGCCCGTCACAAAGAGCTTCAACAGGGGAGAGTGT |
| | | AGCGGAGGAGGAGGAAGCGGAGGAGGAGGAAGCGCCCCCCTCAAAATCCAAGC |
| | | GTACTTCAACGAAACTGCAGACTTACCGTGTCAGTTTGCCAATTCGCAGAATC |
| | | TGAGCCTGAGCGAACTGGTGGTTTTCTGGCAGGATCAGGAGAACCTGGTTCTG |
| | | AACGAAGTCTATCTGGGCAAAGAGCGGTTCGACAGCGTGGACAGCAAGTATAT |
| | | GGGCCGCACCAGCTTTGATAGCGACAGCTGGACCCTGCGTCTGCACAATCTGC |
| | | AAATCAAAGATAAGGGTAGGTACCAGTGCATTATCCACCATAAGAAGCCGACG |
| | | GGTATGATTAATATTCACCAAATGAACTCCGAGTTGTCTGTCCTGGCG |
| 118 | Bispecific | GACATCCAGATGACCCAGTCTCCATCCTCCCTGAGCGCATCTGTAGGAGACCG |
| | molecule | CGTCACCATCACTTGCCGGGCAAGTCAGAGCATTAGCAGCTATTTAAATTGGT |
| | 1140/1141: | ATCAGCAGAAACCAGGGAAAGCCCCTAAGCTCCTGATCTATGCTGCATCCAGT |
| | clone 1141 | TTGCAAAGTGGGGTCCCATCACGTTTCAGTGGCAGTGGAAGCGGGACAGATTT |
| | light | CACTCTCACCATCAGCAGTCTGCAACCTGAAGATTTTGCAACTTATTACTGTC |
| | chain, | AACAGAGTTACAGTACCCCTTATACTTTTGGCCAGGGGACCAAGCTGGAGATC |
| | VL-kappa, | AAACgtgagtcgtacgctagcaagcttgatatcgaattctaaactctgagggg |
| | Linker | gtcggatgacgtggccattctttgcctaaagcattgagtttactgcaaggtca |
| | SGGGGSGGGGS, | gaaaagcatgcaaagccctcagaatggctgcaaagagctccaacaaaacaatt |
| | 1040: CD86 | tagaactttattaaggaatagggggaagctaggaagaaactcaaaacatcaag |
| | mutant | attttaaatacgcttcttggtctccttgctataattatctgggataagcatgc |
| | molecule | tgttttctgtctgtccctaacatgccctgtgattatccgcaaacaacacaccc |
| | | aagggcagaactttgttacttaaacaccatcctgtttgcttctttcctcagGA |
| | | ACTGTGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAGTCA |
| | | CCCATCAGGGCCTGAGCTCGCCCGTCACAAAGAGCTTCAACAGGGGAGAGTGT |
| | | AGCGGAGGAGGAGGAAGCGGAGGAGGAGGAAGCGCCCCCCTCAAAATCCAAGC |
| | | GTACTTCAACGAAACTGCAGACTTACCGTGTCAGTTTGCCAATTCGCAGAATC |
| | | TGAGCCTGAGCGAACTGGTGGTTTTCTGGCAGGATCAGGAGAACCTGGTTCTG |
| | | AACGAAGTCTATCTGGGCAAAGAGCGGTTCGACAGCGTGGACAGCAAGTATAT |
| | | GGGCCGCACCAGCTTTGATAGCGACAGCTGGACCCTGCGTCTGCACAATCTGC |
| | | AAATCAAAGATAAGGGTAGGTACCAGTGCATTATCCACCATAAGAAGCCGACG |
| | | GGTATGATTAATATTCACCAAATGAACTCCGAGTTGTCTGTCCTGGCG |
| 119 | Bispecific | GACATCCAGATGACCCAGTCTCCATCCTCCCTGAGCGCATCTGTAGGAGACCa |
| | molecule | CGTCACCATCACTTGCCGGGCAAGTCAGAGCATTAGCAGCTATTTAAATTGGT |
| | 1151/1150: | ATCAGCAGAAACCAGGGAAAGCCCCTAAGCTCCTGATCTATGCTGCATCCAGT |
| | clone 1151 | TTGCAAAGTGGGGTCCCATCACGTTTCAGTGGCAGTGGAAGCGGGACAGATTT |
| | light | CACTCTCACCATCAGCAGTCTGCAACCTGAAGATTTTGCAACTTATTACTGTC |
| | chain, | AACAGTACGGTTCTGCTCCGCCCACTTTTGGCCAGGGGACCAAGCTGGAGATC |
| | VL-kappa, | AAACgtgagtcgtacgctagcaagcttgatatcgaattctaaactctgagggg |
| | Linker | gtcggatgacgtggccattctttgcctaaagcattgagtttactgcaaggtca |
| | SGGGGSGGGGS, | gaaaagcatgcaaagccctcagaatggctgcaaagagctccaacaaaacaatt |
| | 1040: CD86 | tagaactttattaaggaatagggggaagctaggaagaaactcaaaacatcaag |
| | mutant | attttaaatacgcttcttggtctccttgctataattatctgggataagcatgc |
| | molecule | tgttttctgtctgtccctaacatgccctgtgattatccgcaaacaacacaccc |
| | | aagggcagaactttgttacttaaacaccatcctgtttgcttctttcctcagGA |
| | | ACTGTGGCTGCACCATCTGTCTTCATCTTCCCGCCATCTGATGAGCAGTTGAA |
| | | ATCTGGAACTGCCTCTGTTGTGTGCCTGCTGAATAACTTCTATCCCAGAGAGG |
| | | CCAAAGTACAGTGGAAGGTGGATAACGCCCTCCAATCGGGTAACTCCCAGGAG |
| | | AGTGTCACAGAGCAGGACAGCAAGGACAGCACCTACAGCCTCAGCAGCACCCT |
| | | GACGCTGAGCAAAGCAGACTACGAGAAACACAAAGTCTACGCCTGCGAAGTCA |
| | | CCCATCAGGGCCTGAGCTCGCCCGTCACAAAGAGCTTCAACAGGGGAGAGTGT |
| | | AGCGGAGGAGGAGGAAGCGGAGGAGGAGGAAGCGCCCCCCTCAAAATCCAAGC |
| | | GTACTTCAACGAAACTGCAGACTTACCGTGTCAGTTTGCCAATTCGCAGAATC |
| | | TGAGCCTGAGCGAACTGGTGGTTTTCTGGCAGGATCAGGAGAACCTGGTTCTG |
| | | AACGAAGTCTATCTGGGCAAAGAGCGGTTCGACAGCGTGGACAGCAAGTATAT |
| | | GGGCCGCACCAGCTTTGATAGCGACAGCTGGACCCTGCGTCTGCACAATCTGC |
| | | AAATCAAAGATAAGGGTAGGTACCAGTGCATTATCCACCATAAGAAGCCGACG |
| | | GGTATGATTAATATTCACCAAATGAACTCCGAGTTGTCTGTCCTGGCG |

A suitable polynucleotide sequence may alternatively be a variant of one of these specific polynucleotide sequences. For example, a variant may be a substitution, deletion or addition variant of any of the above nucleic acid sequences. A variant polynucleotide may comprise 1, 2, 3, 4, 5, up to 10, up to 20, up to 30, up to 40, up to 50, up to 75 or more nucleic acid substitutions and/or deletions from the sequences given in the sequence listing.

Suitable variants may be at least 70% homologous to a polynucleotide of any one of nucleic acid sequences disclosed herein, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example the UWGCG Package provides the BESTFIT program which can be used to calculate homology (e.g. used on its default settings) (Devereux et al (1984) Nucleic Acids Research 12, p387-395).

The PILEUP and BLAST algorithms can also be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analysis is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes, and is capable of expressing, it.

Polynucleotides of the invention can be synthesised according to methods well known in the art, as described by way of example in Sambrook et al (1989, Molecular Cloning—a laboratory manual; Cold Spring Harbor Press).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al.

The invention also includes cells that have been modified to express a polypeptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for an polypeptide of the invention include mammalian HEK293T, CHO, HeLa, NSO and COS cells. Preferably the cell line selected will be one which is not only stable, but also allows for mature glycosylation and cell surface expression of a polypeptide.

Such cell lines of the invention may be cultured using routine methods to produce an polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

### Pharmaceutical Formulations, Therapeutic uses and Patient Groups

In another aspect, the present invention provides compositions comprising molecules of the invention, such as the polypeptides, polynucleotides, vectors and cells described herein. For example, the invention provides a composition comprising one or more molecules of the invention, such as one or more polypeptides of the invention, and at least one pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for parenteral, e.g. intravenous, intramuscular or subcutaneous administration (e.g., by injection or infusion). Depending on the route of administration, the polypeptide may be coated in a material to protect the polypeptide from the action of acids and other natural conditions that may inactivate or denature the polypeptide.

Preferred pharmaceutically acceptable carriers comprise aqueous carriers or diluents. Examples of suitable aqueous carriers that may be employed in the compositions of the invention include water, buffered water and saline. Examples of other carriers include ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition.

A composition of the invention also may include a pharmaceutically acceptable anti-oxidant. These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Therapeutic compositions typically must be sterile and stable under the conditions of manufacture and storage. The composition can be formulated as a solution, microemulsion, liposome, or other ordered structure suitable to high drug concentration.

Sterile injectable solutions can be prepared by incorporating the active agent (e.g. polypeptide) in the required amount in an appropriate solvent with one or a combination of ingredients enumerated above, as required, followed by sterilization microfiltration. Generally, dispersions are prepared by incorporating the active agent into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active agent plus any additional desired ingredient from a previously sterile-filtered solution thereof.

Particularly preferred compositions are formulated for systemic administration or for local administration. Local administration may be at the site of a tumour or into a tumour draining lymph node. The composition may prefereably be formulated for sustained release over a period of time. Thus the composition may be provided in or as part of a matrix facilitating sustained release. Preferred sustained release matrices may comprise a Montanide or y-Polyglutamic acid (PGA) nanoparticles. Localised release of a polypeptide of the invention, optionally over a sustained period of time, may reduce potential autoimmune side-effects associated with administration of a CTLA-4 antagonist.

Compositions of the invention may comprise additional active ingredients as well as a polypeptide of the invention. As mentioned above, compositions of the invention may comprise one or more polypeptides of the invention. They may also comprise additional therapeutic or prophylactic agents.

Also within the scope of the present invention are kits comprising polypeptides or other compositions of the invention and instructions for use. The kit may further contain one ore more additional reagents, such as an additional therapeutic or prophylactic agent as discussed above.

The polypeptides in accordance with the present invention maybe used in therapy or prophylaxis. In therapeutic applications, polypeptides or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, polypeptides or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means.

In particular, polypeptides of the invention may be useful in the treatment or prevention of cancer. Accordingly, the invention provides a polypeptide of the invention for use in the treatment or prevention of cancer. The invention also provides a method of treating or preventing cancer comprising administering to an individual a polypeptide of the invention. The invention also provides an polypeptide of the invention for use in the manufacture of a medicament for the treatment or prevention of cancer.

The cancer may be prostate cancer, breast cancer, colorectal cancer, pancreatic cancer, ovarian cancer, lung cancer, cervical cancel, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

A polypeptide of the present invention, or a composition comprising said polypeptide, may be administered via one or more routes of administration using one or more of a variety of methods known in the art. As will be appreciated by the skilled artisan, the route and/or mode of administration will vary depending upon the desired results. Systemic administration or local administration are preferred. Local administration may be at the site of a tumour or into a tumour draining lymph node. Preferred modes of administration for polypeptides or compositions of the invention include intravenous, intramuscular, intradermal, intraperitoneal, subcutaneous, spinal or other parenteral modes of administration, for example by injection or infusion. The phrase “parenteral administration” as used herein means modes of administration other than enteral and topical administration, usually by injection. Alternatively, an polypeptide or composition of the invention can be administered via a non-parenteral mode, such as a topical, epidermal or mucosal mode of administration.

A suitable dosage of an polypeptide of the invention may be determined by a skilled medical practitioner. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular polypeptide employed, the route of administration, the time of administration, the rate of excretion of the polypeptide, the duration of the treatment, other drugs, compounds and/or materials used in combination with the particular compositions employed, the age, sex, weight, condition, general health and prior medical history of the patient being treated, and like factors well known in the medical arts.

A suitable dose of an polypeptide of the invention may be, for example, in the range of from about 0.1 μg/kg to about 100 mg/kg body weight of the patient to be treated. For example, a suitable dosage may be from about 1 μg/kg to about 10 mg/kg body weight per day or from about 10 g/kg to about 5 mg/kg body weight per day.

Dosage regimens may be adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Polypeptides may be administered in a single dose or in multiple doses. The multiple doses may be administered via the same or different routes and to the same or different locations. Alternatively, polypeptides can be administered as a sustained release formulation as described above, in which case less frequent administration is required. Dosage and frequency may vary depending on the half-life of the polypeptide in the patient and the duration of treatment that is desired. The dosage and frequency of administration can also vary depending on whether the treatment is prophylactic or therapeutic. In prophylactic applications, a relatively low dosage may be administered at relatively infrequent intervals over a long period of time. In therapeutic applications, a relatively high dosage may be administered, for example until the patient shows partial or complete amelioration of symptoms of disease.

Combined administration of two or more agents may be achieved in a number of different ways. In one embodiment, the polypeptide and the other agent may be administered together in a single composition. In another embodiment, the polypeptide and the other agent may be administered in separate compositions as part of a combined therapy. For example, the modulator may be administered before, after or concurrently with the other agent.

A polypeptide or composition of the invention may also be used in a method of increasing the activation of a population of cells expressing human CD40 and human CTLA-4, the method comprising administering to said population of cells a polypeptide or composition of the invention under conditions suitable to permit interaction between said cell and a polypeptide of the invention. The population of cells typically comprises at least some cells which express CTLA-4, typically T cells, and at least some cells which express CD40, typically an antigen presenting cell such as a B cell. The method is typically carried out ex vivo.

The present invention is further illustrated by the following examples which should not be construed as further limiting. The contents of all figures and all references, patents and published patent applications cited throughout this application are expressly incorporated herein by reference.

### EXAMPLES

### Examples 1 to 4—Development of Polypeptide Variants of CD86 for Part B2

### Example 1—First Selection

### Introduction

The starting material for the production of the polypeptide variants was the monomeric soluble extracellular binding domain of human CD86. That is, the CTLA-4 binding domain of human CD86. The amino acid sequence of this domain and a structural model of CD86 in complex with CTLA-4 (Schwartz et al; Nature 2001; 410(6828) p604-608) was used to design 4 different starting phage display libraries of candidate polypeptides: AL-1014-01, AL-1014-02, AL-1014-03 and AL-1014-04. The phage display libraries were produced using standard protocols using nucleotide sequences encoding the candidate polypeptides. The amino acid sequences of the candidate polypeptides were designed as set out below.

### Library Design

The primary purpose for the design of library AL-1014-01 was to provide an increased binding surface of the binding domain of CD86 for the interaction with CTLA-4. To this end, various residues in the FG loop of CD86 (positions 114 to 121, numbering as in FIG. 4) were selected for mutation. Two insertions were also introduced to allow for a elongation of the FG-loop. The positions and introduced mutations relative to the wild-type sequence of CD86 are summarised in Table 1 below. The variability that was allowed in each position is displayed. Nucleotides encoding all of the possible polypeptides which result from all of the possible combinations of the mutations shown in Table 1 were designed and used to produce the AL-1014-01 phage display library, in accordance with standard protocols.

TABLE 1

| Amino acid sequence number according to SEQ ID NO: 1 | | | | | | | | | | | | |
|---|---|---|---|---|---|---|---|---|---|---|---|---|
| 113 | 114 | 115 | 116 | insert | 117 | 118 | insert | 119 | 120 | 121 | 122 | |
| H | H | K | K | | P | T | | G | M | I | R | Wt- |
| | | | | | | | | | | | | sequence |
| Y | Y | Y | Y | Y | Y | Y | Y | Y | Y | Y | Y | Amino |
| S | S | S | S | S | S | S | S | S | S | S | S | acids |
| G | G | G | G | G | G | G | G | | G | G | G | allowed |
| L | L | L | L | L | L | L | L | L | L | L | L | in each |
| D | D | D | D | D | D | D | D | D | D | D | D | position |
| | K | | | | R | K | | | | K | | (restricted) |
| | | | | | | | | | | | | variability) |

In library AL-1014-02 amino acid positions in the binding surfaces of CD86 facing CTLA-4 were allowed to vary between the wild type amino acid and a very limited number of homologous residues to introduce minimal structural perturbations in the beta-sheet surface of CD86. The positions and introduced mutations of the library AL-1014-02 are shown in Table 2. A mutation in position 79 has been reported to contribute favorably to the binding of CD86 to CTLA-4 (Peach et al; Journal of Biological Chemistry 1995; 270 p21181-21187), and this position was also allowed to vary in the library. The variability that was allowed in each position is displayed. Nucleotides encoding all of the possible polypeptides which result from all of the possible combinations of the mutations shown in Table 2 were designed and used to produce the AL-1014-02 phage display library, in accordance with standard protocols. Table 2 also shows the IUPAC-IUB code that was used to represent the indicated mutations in the corresponding nucleotide sequences. IUPAC-IUB codes are typically used to define multiple nucleotide possibilities in a given position and are based on a recognised set of nomeclature rules for incompletely specified bases in nucleotide sequences (see Biochem. J., 1985, 229, 281-286; Eur. J. Biochem., 1985, 150, 1-5; J. Biol. Chem., 1986, 261, 13-17; Mol. Biol. Evol., 1986, 3, 99-108; Nucl. Acids Res., 1985, 13, 3021-3030; Proc. Nat. Acad. Sci. (U. S.), 1986, 83, 4-8; and Biochemical Nomenclature and Related Documents 2nd edition, Portland Press, 1992, pp 122-126). The IUPAC-IUB codes used in these experiments are summarised below Table 2.

TABLE 2
Amino acid sequence position in human CD86-numbering as in FIG. 4

| | | | | | | | | | | | | | | | | | | | | | |
|---|---|---|---|---|---|---|---|---|---|---|---|---|---|---|---|---|---|---|---|---|---|
| saa | rtt | Twc | Saa | rtt | gam | Twc | arg | kct | san | htc | Saa | Vtc | san | asc | gst | mtg | rtt | arg | saa | rac | IUB |
| | | | | | | | | | | | | | | | | | | | | | code |
| 48 | 54 | 56 | 58 | 64 | 67 | 69 | 74 | 77 | 79 | 107 | 109 | 111 | 113 | 118 | 119 | 120 | 121 | 122 | 125 | 127 | a.a. |
| | | | | | | | | | | | | | | | | | | | | | position |
| Q | V | F | Q | V | E | Y | K | S | H | L | Q | I | H | T | G | M | I | R | Q | N | Wt-sequ |
| E | I | Y | E | I | D | F | R | A | D | I | E | L | D | S | A | L | V | K | E | D | Amino acids |
| | | | | | | | | | E | F | | V | E | | | | | | | | allowed in |
| | | | | | | | | | Q | | | | Q | | | | | | | | each position |
| | | | | | | | | | A | | | | A | | | | | | | | (restricted |
| | | | | | | | | | | | | | | | | | | | | | variability) |

IUPAC-IUB codes

| IUB Code | N | V | B | H | D | K | S | W | M | Y | R |
|---|---|---|---|---|---|---|---|---|---|---|---|
| Nucleotide | A, C, | G, A, C | G, T, C | A, T, C | G, A, T | G, T | G, C | A, T | A, C | C, T | A, G |
| bases | G, T | | | | | | | | | | |

Two more libraries were designed. In these two libraries, the entire IgGV domain of CD86 was randomized by taking the nucleotide sequence encoding wild type CD86 and using an error prone PCR method set-up to minimize mutational bias. The resulting mutant nucleotide sequences were used to produce phage display libraries in accordance with standard protocols.

Three different random mutagenized libraries were generated of which two were selected. AL-1014-03 was taken from randomization round 2 and randomization round 3 provided library AL-1014-04.

AL-1014-1, AL-1014-2, AL-1014-3 and AL-1014-04 were produced by cloning into a phagemid vector and phage stocks were generated according to standard protocols.

### Selection Strategy

The selection strategy applied in this Example consisted of 6 rounds (R1 to R6) which are summarised in Table 3.

TABLE 3

| Selection | | | Wash | Non | | | |
|---|---|---|---|---|---|---|---|
| round | Ag conc | Incubation (mm) | (mm) | bio-Ag | Temp | pH | CD28 |
| R1 | 50 nM | 60 (with hCTLA4-bio) + | Standard | | | | |
| | | 30 (with beads) | | | | | |
| R2 | 5 nM | 20 (with hCTLA4-bio) + | Standard | | | | 300 nM * |
| | | 30 (with beads) | | | | | |
| R3 | 20 nM | 60 (with mCTLA4-bio) + | Standard | | | | |
| | mCTLA4 | 30 (with beads) | | | | | |
| R4 | 5 nM | 10 (with hCTLA4-bio) + | Standard + | | 60° C., | | 200 nM ** |
| | | 20 (with beads) | 10 | | 3 hours | | |
| R5 | 5 nM | 5 (with hCTLA4-bio) + | Standard + | | | | 200 nM** |
| | | 10 (non-bio CTLA4) + | 30 | | | | |
| | | 20 (with beads) | | | | | |
| R6 | 2 nM | 5 (with hCTLA4-bio) + | Standard + | X | | pH 6.3 | 200 nM** |
| | | 10 (non-bio CTLA4) | 120 | | | pH 5.5 | |
| | | (*1000) + 15 (with beads) | | | | | |

*Pre-selection with CD28-Fc
**CD28-Fc added at the same time as bio-CTLA4

The first round of selection (R1) enriched the members of starting libraries AL-1014-01, AL-1014-02, AL-104-03 and AL-1014-04 for binders to biotinylated CTLA4-Fcγ (50 nM). The enrichment for functional binders was confirmed by sequencing.

The output from R1 was a pool of phage clones from each starting library, enriched for functional binders. The output from libraries AL-104-03 and AL-1014-04 was subsequently combined into a new library AL-1014-05. The output from libraries AL-1014-01, AL-1014-02 and AL-1014-04 was combined into a further new library, AL-1014-06. Members of libraries AL-1014-05 and AL-1014-06 were then subjected to 5 additional rounds of screening (R2 to R6), to select for increased binding to CTLA-4 and decreased binding to CD28.

The strategy for the additional 5 rounds of selection was to apply selection pressure specificially aimed at improving the properties of affinity, on-rate, off-rate, selectivity, multimerization and epitope maintenance.

Selection for increased affinity was achieved by lowering the antigen concentration in each round. The selections started at 50 nM CTLA-4 lowering to 5 nM which was maintained in rounds R2, R4 and R5, followed by a final round (R6) in which the CTLA-4 concentration was 2 nM. Selection for increased on-rate was achieved by shortening of incubation time with biotinylated CTLA-4. Selection for decreased off-rate was achieved by increasing the stringency during the wash step. Selection for increased selectivity for CTLA-4 (and reduced binding affinity to CD28) was achieved by incubating in excess of unbiotinylated CD28-Fc (in R2, R4, R5 and R6). Retained binding affinity to mouse CTLA-4 was ensured by including a round (R3) in which biotinylated murine CTLA-4 was used in place of human CTLA-4 as the selection antigen. This step was included to make sure that the affinity to mouse CTLA4 was kept to enable proof-of-concept experiments in mice models. Selecting for avidity effects was avoided by introducing unbiotinylated CTLA-4 five minutes after the start of incubation with biotinylated CTLA-4 (in R5 and R6). Addition of unbiotinylated Fcγ (IgG) to the selection buffer was performed to avoid selecting for binders to the Fc-fusion part of the CTLA-4 antigen.

Decreased temperature sensitivity and potentially increased melting point was obtained by introducing a selection step at increased temperature (60° C.) (R4). Increased affinity at low pH was addressed by introducing a selection round at a lower pH (R6).

After the selection rounds individual phage clones from R5 and R6 were analysed by high throughput screening in a conventional affinity ELISA assay. The assay was a sandwich ELISA which measured binding of phages to either CTLA-4 or CD28. In short, 96-well flat bottom high binding plates (Greiner #655074) were coated with either CTLA4-Fcγ (Orencia, Apoteket) or CD28-Fcγ (R&D systems 342-CD) by incubating overnight at 4° C. The plates were washed (Wash buffer: PBS+0.05% Tween 20 (PBST) Medicago #09-9410-100) and then blocked in PBST+3% Milk powder (Semper). The plates were washed again and sample or controls were added to the wells. The samples were incubated for 1 h at room temperature and then washed. Detection antibody, mouse-anti-M13-HRP (GE Healthcare, #27-9421-01) was added and the plates were subsequently developed using SuperSignal Pico Chemiluminescent substrate (Thermo #37069) and detected with an Envision reader (Perkin Elmer).

### Example 2—Expression and Recloning of Selected Polypeptides

The phage clones selected from Example 1 were re-cloned according to standard protocols into a fusion protein-format, with each clone fused to γ2/γ4 Fc. The fusion proteins were expressed in HEK293 cells. Supernatants culure of the cells were collected and the expressed fusion proteins were assayed using both ELISA and Biacore™ according to the methods set out below. The results are shown in Table 4, which also shows the mutations present in each clone. The best performing clone in the ELISA assay (907) had an EC50 binding ratio almost 10 times higher than wild-type protein CD86. The best performing clone in the Biacore™ assay (904) had a Kd binding ratio more than 4 times higher than wild type protein CD86.

### Binding ELISA

96-well flat bottom high binding plates (Greiner #655074) were coated with either CTLA4-Fc (Fitzgerald #30R-CD152) or CD28-Fc (R&D systems 342-CD) by incubating overnight at 4° C. The plates were washed (Wash buffer: PBS+0.05% Tween 20 (PBST) Medicago #09-9410-100) and then blocked in PBST+3% BSA (Merck, #1.12018.0100).

The plates were washed again and sample or controls (serially diluted 1/5 from 200-0.001 μg/ml) were added to the wells. The samples were incubated for 1 h at room temperature and then washed. Detection antibody, goat-anti-human IgG Fcγ-HRP (Jackson, #109-035-098) was added and the plates were subsequently developed using SuperSignal Pico Chemiluminescent substrate (Thermo #37069) and detected with an Envision reader

(Perkin Elmer). EC50 values were calculated for both CTLA4 and CD28. The binding ratio (EC50 binding ratio=[EC50 for CD28]÷[EC50 for CTLA-4]) was calculated for each polypeptide and is shown in Table 4.

### Biacore™

Either CTLA4-Fc (Fitzgerald #30R-CD152) or CD28-Fc (R&D systems 342-CD) was immobilized to the Biacore™ senshorship, CMS, using conventional amine coupling. The CD86 mutant molecules and controls (serially diluted 1/2 100-1.5 nM) were analyzed for binding in HBs-P (GE, BR-1003-69) at a flow rate 30 μ1/ml. The association was followed for 3 minutes and the dissociation for 10 minutes. Regeneration was performed twice using 5 mM NaOH for 30 seconds. The kinetic parameters and the affinity constants were calculated using BlAevaluation 4.1 software. The binding ratio (Kd binding ratio=[Kd for CD28]÷[Kd for CTLA-4]) was calculated for each polypeptide and is shown in Table 4.

TABLE 4

| | | EC50 | Kd |
|---|---|---|---|
| | Mutated positions and amino acid change | binding | binding |
| Clone | (positions numbered as in FIG. 4) | ratio | ratio |
| 900 | H79D, L107I, I111V, T118S, M120L, I121V, | 3.5 | ND* |
| | R122K, Q125E | | |
| 901 | Q48L, S49T, L107I, I111V, R122K, Q125E, | 17.2 | 2.7 |
| | A134T | | |
| 904 | V54I, K74R, S77A, H79D, L107I, T118S, | 12.2 | 6.8 |
| | I121V, R122K, N127D | | |
| 906 | F32I, Q48L, K74R, H79D, L107F, M120L, | 16.2 | 0.8 |
| | I121V, R122K, Q125E | | |
| 907 | R122K, Q125E | 30.5 | 5.6 |
| 908 | L107I, I121V, R122K, Q125E | 6.2 | 4.7 |
| 910 | H79D, L107I, I121V, R122K, Q125E | 7.7 | 5.1 |
| 915 | V64I, H79D, L107F, T118S, M120L, R122K, | 9.9 | 1.9 |
| | N127D | | |
| 938 | V64I, L107I, I121V, R122K | 2.0 | 5.5 |
| | Wild type | 3.4 | 1.6 |

*No detectable binding was seen in the BIAcore analysis

Table 5 summarises the mutations and positions in each of the selected clones. The full amino acid sequences for clones 900, 901, 904, 906, 907, 908, 910, 915 and 938 are provided as SEQ ID NOs: 6 to 14, respectively.

TABLE 5

| | F32I | Q48L | S49T | V54I | V64I | K74R | S77A | H79D | L107I/F |
|---|---|---|---|---|---|---|---|---|---|
| 900 | | | | | | | | D | I |
| 901 | | L | T | | | | | | I |
| 904 | | | | I | | R | A | D | I |
| 906 | I | L | | | | R | | D | F |
| 907 | | | | | | | | | |
| 908 | | | | | | | | | I |
| 910 | | | | | | | | D | I |
| 915 | | | | | I | | | D | F |
| 938 | | | | | I | | | | I |

| | I111V | T118S | M120L | I121V | R122K | Q125E | N127D | A134T |
|---|---|---|---|---|---|---|---|---|
| 900 | V | x | L | v | K | E | | |
| 901 | V | | | | K | E | | T |
| 904 | | S | | v | K | | D | |
| 906 | | | L | v | K | E | | |
| 907 | | | | | K | E | | |
| 908 | | | | v | K | E | | |
| 910 | | | | v | K | E | | |
| 915 | | S | L | | K | | D | |
| 938 | | | | v | K | | | |

### Example 3—Expanded Library Diversity and Repeated Selection and Screening

### Library Production

The starting material for two further libraries, AL-1014-11 and AL-1014-12, were six clones identified in Examples 1 and 2, namely 901, 904, 906,907, 908, 915. Additional diversity was generated by error-prone PCR with mutated oligos included in the reassembly PCR step, in accordance with protocols described in WO2002048351, WO200309734, and WO2007057682. The applied oligos comprised mutations in hotspot regions of the CD86 molecules. Approximately 20 clones from each library were sequenced. The two libraries contained recombined clones, error prone PCR generated clones, and clones produced by the introduction of mutated oligos. Each clone contained on average 3-11 mutations compared to the wild-type sequence of CD86.

### Selection Strategy

Selection rounds R2 to R6 as described in Example 1 were applied to both libraries AL-1014-11 and AL-1014-12. Clones selected in the last two rounds were sequenced to confirm that recombination and novel mutations were achieved.

### Assesment of Selected Clones

Approximately 1250 clones from the last two selection rounds were expressed as phage stocks and analyzed for binding to CTLA-4 and CD28 by the same high throughput screening ELISA as described in Example 1 (data not shown). Clones were ranked based on their binding to CTLA-4 and CD28. The top ten clones were selected based on the criteria of high binding to CTLA-4 and low binding to CD28. The sequence of these clones was determined and each was expressed from HEK293 cells as a gamma2/gamma4 fusion, as described in Example 2.

Table 6 summarises the mutations and positions in each of the top ten clones. The full amino acid sequences for clones 1038, 1039, 1040, 1041, 1042, 1043, 1044, 1045, 1046 and 1047 are provided as SEQ ID NOs: 15 to 24, respectively.

TABLE 6

| | F32L | Q48L | S49T | K74I/R | H79D/S/A | K103E | L107I/R |
|---|---|---|---|---|---|---|---|
| 1038 | | L | | | D | | I |
| 1039 | | L | | | S | | I |
| 1040 | | L | | R | D | | R |
| 1041 | L | L | | | D | | I |
| 1042 | | L | | I | S | | I |
| 1043 | | L | | | D | | I |
| 1044 | | L | T | | S | | I |
| 1045 | | L | T | | D | | |
| 1046 | | | | | D | E | I |
| 1047 | | L | | | D | | I |

| | T118S | M120L | I121V | R122K/N | Q125E | N127S/D |
|---|---|---|---|---|---|---|
| 1038 | | | V | K | E | |
| 1039 | S | | V | K | | D |
| 1040 | | | | N | | |
| 1041 | | L | V | K | E | |
| 1042 | S | | V | K | | D |
| 1043 | | | | K | E | |
| 1044 | | | | K | E | S |
| 1045 | | L | V | K | E | |
| 1046 | S | | V | K | | D |
| 1047 | | L | V | K | E | |

### Example 4—Further Assessment of Top Ten Polypeptides

The polypeptides from each of the ten clones selected in Example 3 were further characterised as follows.

### Expression and Purification

Plasmids encoding each clone were transfected into Freestyle 293-T cells (Invitrogen) according to standard protocols and supernatant were harvested on day 6.

Polypeptides and controls were affinity purified using protein A columns (GE Healthcare #17-0402-01).

### Binding ELISA

Similar to the protocol described in Example 2, 96-well flat bottom high binding plates (Greiner #655074) were coated with either CTLA4-Fc (Fitzgerald #30R-CD152) or CD28-Fc (kindly provided by Simon Davis, Oxford University) by incubating overnight at 4° C. The plates were washed (Wash buffer: PBS+0.05% Tween 20 (PBST) Medicago #09-9410-100) and then blocked in PBST+3% BSA (Merck, #1.12018.0100). The plates were washed again and sample or controls (serially diluted 1/3 from 50 000-0001 nM) were added to the wells. The samples were incubated for 1 h at room temperature and then washed. Detection antibody, goat-anti-human IgG Fγy-HRP (Jackson, #109-035-098) was added and the plates were subsequently developed using SuperSignal Pico Chemiluminescent substrate (Thermo #37069) and detected with an Envision reader (Perkin Elmer).

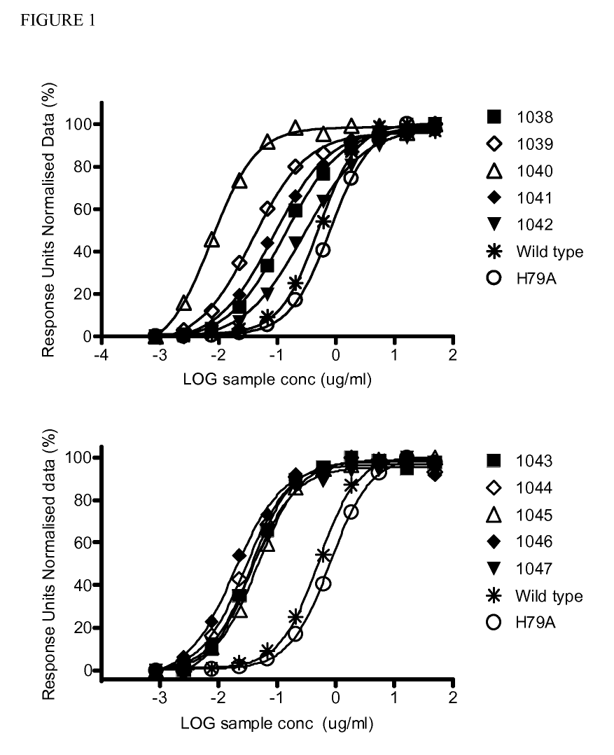

The results of the CTLA-4 binding ELISA are shown in FIG. 1. EC50 values were calculated and are shown in Table 7. All molecules showed better EC50 values than both wild type and H79A, with an improvement of between 2 and 67-fold. Binding to CD28 was too low for detection (data not shown).

TABLE 7

| Sample | EC50 |
|---|---|
| 1038 | 0.14 |
| 1039 | 0.039 |
| 1040 | 0.0076 |
| 1041 | 0.087 |
| 1042 | 0.29 |
| 1043 | 0.035 |
| 1044 | 0.029 |
| 1045 | 0.047 |
| 1046 | 0.019 |
| 1047 | 0.037 |
| Wild type | 0.51 |
| Prior Art | 0.81 |
| Negative control | No activity |

### Inhibition ELISA

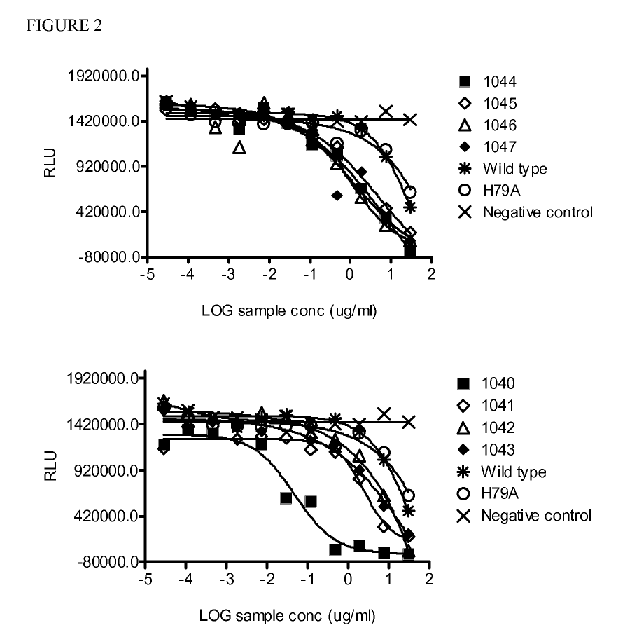

96-well flat bottom plate high binding plates (Greiner #655074) were coated with wildtype CD86-Fc (R&D Systems #7625-B2) by incubating overnight at 4° C. The plates were washed (Wash buffer: PBS+0.05% Tween 20 (PBST) Medicago #09-9410-100) and then blocked in PBST+3% BSA (Merck, #1.12018.0100). The sample (CD86 mutant or wild type protein; serially diluted 1/4 from 30000 to 0.3 ng/ml) was incubated with biotinylated-CTLA4 (Fitzgerald #30R-CD152) in room temperature 1 h, the mixture was then added to the blocked wells in the ELISA plate. Detection was performed with Streptavidin-HRP (Pierce #21126) and the plates were subsequently developed using SuperSignal Pico Chemiluminescent substrate (Thermo #37069) and detected with Envision reader (Perkin Elmer). The results are shown in FIG. 2. IC50 values were calculated and are shown in Table 8. All molecules showed better IC50 value than both wild type and H79A, the IC of the best mutant CD86 molecule was improved over 100 fold compared to wild type.

TABLE 8

| Sample | IC50 |
|---|---|
| 1040 | 0.049 |
| 1041 | 3.1 |
| 1042 | 4.3 |
| 1043 | 4.0 |
| 1044 | 1.4 |
| 1045 | 2.6 |
| 1046 | 1.1 |
| 1047 | 0.98 |
| Wild type | 15 |
| H79A | 25 |
| Negative control | No activity |

### Biacore™

As in Example 2, either CTLA4-Fc (Fitzgerald #30R-CD152) or CD28-Fc (kindly provided by Simon Davis, Oxford University)was immobilized to the Biacore™ senshorship, CMS, using conventional amine coupling. The CD86 mutant molecules and controls (serially diluted 1/3 1000-4 nM) were analyzed for binding in HBs-P (GE, BR-1003-69) at a flow rate 30 μ/ml. The association was followed for 3 minutes and the dissociation for 10 minutes. Regeneration was performed twice using 5 mM NaOH for 30 seconds. The kinetic parameters and the affinity constants were calculated using BIAevaluation 4.1 software.

The CTLA-4 results for each selected clone and wild type CD86 to CTLA-4 are summarised in Table 9. The mutations present in each clone are also shown. A clone including only the H79A mutation from Peach et al (Journal of Biological Chemistry 1995, vol 270(36), 21181-21187) is also included for comparison. The CD28 results are not shown, since in most cases affinity to CD28 was too weak to be determined by this protocol. However, where it was determined, the affinity of the selected CD86 clones for CD28 was at least 100 times lower than that for CTLA-4.

It was also determined that the selected clones also bind to murine CTLA-4 (data not shown).

TABLE 9

| | Mutated positions and amino acid change | ka | kd | Kd |
|---|---|---|---|---|
| Clone | (positions numbered as in FIG. 4) | (1/Ms) | (1/s) | (nM) |
| 1038 | Q48L, H79D, L107I, I121V, R122K, Q125E | 1.0e6 | 0.012 | 12 |
| 1039 | Q48L, H79S, L107I, T118S, I121V, R122K, N127D | 1.0e6 | 8.5e−3 | 8 |
| 1040 | Q48L, K74R, H79D, L107R, R122N | 1.0e6 | 3.2e−3 | 3 |
| 1041 | F32L, Q48L, H79D, L107I, M120L, I121V, R122K, 125E | 7.0e5 | 8.4e−3 | 12 |
| 1042 | Q48L, K74I, H79S, L107I, T118S, I121V, R122K, N127D | 4.4e5 | 0.011 | 25 |
| 1043 | Q48L, H79D, L107I, R122K, Q125E | 1.1e6 | 0.011 | 10 |
| 1044 | Q48L, S49T, H79S, L107I, R122K, Q125E, N127S | 1.1e6 | 9.4e−3 | 8 |
| 1045 | Q48L, S49T, H79D, M120L, I121V, R122K, Q125E | 9.4e5 | 8.3e−3 | 9 |
| 1046 | H79D, K103E, L107I, T118S, I121V, R122K, N127D | 1.4e6 | 8.0e−3 | 6 |
| 1047 | Q48L, H79D, L107I, M120L, I121V, R122K, Q125E | 8.5e5 | 8.4e−3 | 10 |
| Wild | | 4.6e5 | 0.023 | 50 |
| type | | | | |
| H79A | | 3.4e5 | 0.022 | 63 |

### Example 5—Cloning and Construction of Exemplary Bispecific Molecules

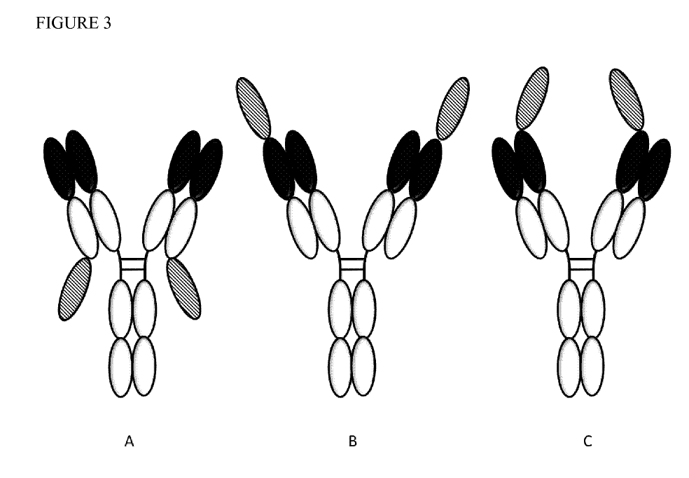

Bispecific polypeptides in accordance with the invention were produced according to the schematic diagram shown in FIG. 3 and as described Table A.

TABLE A

| Reference | |
|---|---|
| no. | Description of molecule |
| 956/530 | Amino acid sequence of 904 (B2; SEQ ID |
| | NO: 8) joined by linker SGGGGSGGGGS |
| | (X; SEQ ID NO: 6) to the C terminal of |
| | the light chain of antibody A2-54 (B1)- |
| | antibody otherwise assembled as normal |
| | with heavy chain of A2-54. |
| 957/530 | Amino acid sequence AP-904 (B2; SEQ ID |
| | NO: 8 with additional AP at N terminus) |
| | joined by linker SGGGGSGGGGS (X; SEQ ID |
| | NO: 46) to the C terminal of the light |
| | chain of antibody A2-54 (B1)-antibody |
| | otherwise assembled as normal with |
| | heavy chain of A2-54. |
| 958/531 | Amino acid sequence of 904 (B2; SEQ ID |
| | NO: 8) joined by linker NFSQP (X; SEQ |
| | ID NO: 48) to the N terminal of the |
| | heavy chain of antibody A2-54 (B1)- |
| | antibody otherwise assembled as normal |
| | with light chain of A2-54. |
| 959/530 | Amino acid sequence of 904 (B2; SEQ ID |
| | NO: 8) joined by linker KRTVA (X; SEQ |
| | ID NO: 49) to the N terminal of the |
| | light chain of antibody A2-54 (B1)- |
| | antibody otherwise assembled as normal |
| | with heavy chain of A2-54. |
| 960/530 | Amino acid sequence of 904 (B2; SEQ ID |
| | NO: 8) joined by linker NFSQP (X; SEQ |
| | ID NO: 48) to the N terminal of the |
| | light chain of antibody A2-54 (B1)- |
| | antibody otherwise assembled as normal |
| | with heavy chain of A2-54. |

Sequences encoding a mutated human CD86 variable soluble domain (B2), a linker (X) and restriction enzyme sites were designed in silico. The sequences were ordered from Eurofins MWG Operon (Ebersberg, Germany) and cloned into a vector containing the heavy or the light chain of a CD40 binding antibody (A2-54; Ellmark et al., Immunology, 108, 452-457, 2003). The vector was then transfected into Freestyle 293 cells (Invitrogen Corp., Carlsbad, US) according to the manufacturer's instructions. The transfected cells were cultivated and harvested. The bispecific constructs produced by the cells were purified on protein A columns (HisTrap protein A, GE Helthcare) according to standard methods.

### Example 6—Characterisation of Exemplary Bispecific Molecules

The isoelectric point of the polypeptides produced in Example 5 was determined using GP-MAW software and is shown below.

| Bispecific antibody | Isoelectric point (pI) |
|---|---|
| 956/530 | 7.8 |
| 957/530 | 7.8 |
| 958/531 | 7.8 |
| 959/530 | 8.7 |
| 960/530 | 7.8 |

Dynamic Light Scattering, DLS, was used to study aggregation behavior and to confirm molecular weights in accordance with standard protocols. No aggregation was observed for two representative polypeptides, 956/530 and 957/530, tested at a concentration of 1 mg/ml. The observed Mw for 956/530 and 957/530 was 265 kDa and 282 kDa, respectively.

Dynamic light scattering was measured using Malvern Zetasizer Nano series ZEN1600 (Worcestershire, UK) and Malvern Low-volume quartz batch cuvette (ZEN2112) according to manufacturer's instructions. 200 μl sterile and dust free pipette tips from Biozym, cat no 720328, were used at each transfer step. Interfering dust particles were removed by centrifugation of the samples for 5 min at 13000 rpm prior to analysis.

### Example 7—Demonstration of Dual Binding Specificity of Exemplary Bispecific Molecules Binding to CTLA-4

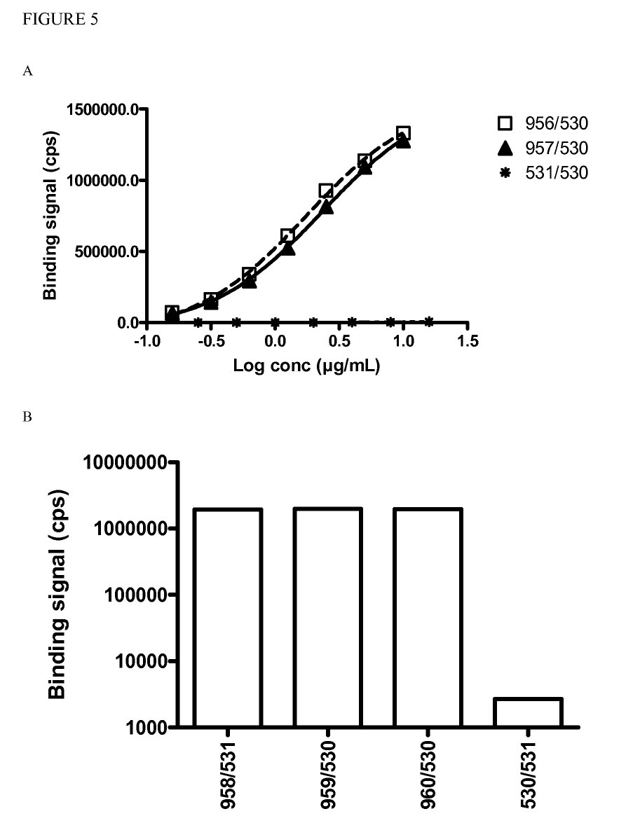

Binding of the polypeptides produced in Example 5 to CTLA-4 was assessed by standard binding ELISA. In short, 96-well flat bottom plate high binding plates (Greiner #655074) were coated with CTLA4-Fc (Fitzgerald #30R-CD152) by incubating overnight at 4° C. The plates were washed (Wash buffer: PBS+0.05% Tween 20 (PBST) Medicago #09-9410-100) and then blocked in PBST+3% BSA (Merck, #1.12018.0100). The plates were washed again and samples or control (serially diluted 1/ from 50 000-0001 nM) were added to the wells. A monospecific antibody for CD40 (530/531: A2-54) was used as the control. The samples were incubated for 1 h at room temperature and then washed. Detection antibody, goat-anti-human IgG Fcγ-HRP (Jackson, #109-035-098) was added and the plates were subsequently developed using SuperSignal Pico Chemiluminescent substrate (Thermo #37069) and detected with Envision reader (Perkin Elmer). Results are shown in FIG. 5. In FIG. 5A the binding of bispecific molecules at different concentrations to CTLA-4 coated onto an ELISA plate is shown. In FIG. 5B, the binding of bispecific molecules at one fixed concentration (100 ng/ml) to CTLA-4 coated onto an ELISA plate is shown. The bi-specific molecules bind specifically to CTLA-4.

### Binding to CD40

Binding of the polypeptides produced in Example 5 to CD40 was assessed by standard binding ELISA. In short, 96-well flat bottom plate high binding plates (Greiner #655074) were coated with CD40-Fc (Ancell # 504-820) by incubating overnight at 4° C. The plates were washed (Wash buffer: PBS+0.05% Tween 20 (PBST) Medicago #09-9410-100) and then blocked in PBST+3% BSA (Merck, #1.12018.0100). The plates were washed again and samples or control (serial dilutions 1/2, starting at 0.2 ug/ml) were added to the wells. The isolated polypeptide of amino acid sequence SEQ ID NO: 8 (soluble CTLA-4 binding domain reference 904) was used as a control as it binds specifically to only CTLA-4.

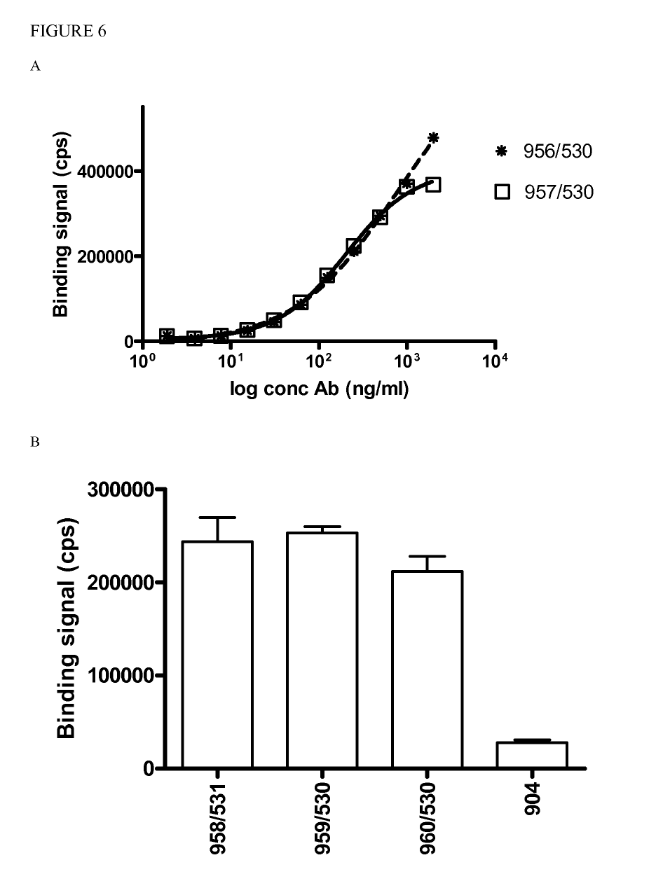

The samples were incubated for 1 h at room temperature and then washed. Detection antibody, goat-anti-human IgG Fcγ-HRP (Jackson, #109-035-098) was added and the plates were subsequently developed using SuperSignal Pico Chemiluminescent substrate (Thermo #37069) and detected with Envision reader (Perkin Elmer). Results are shown in FIG. 6. In FIG. 6A the binding of bispecific molecules at different concentrations to CD40 coated onto an ELISA plate is shown. In FIG. 6B, the binding of bispecific molecules at one fixed concentration (100 ng/ml) to CD40 coated onto an ELISA plate is shown. The bispecific molecules bind specifically to CD40.

### Dual Binding to CD40 and CTLA-4

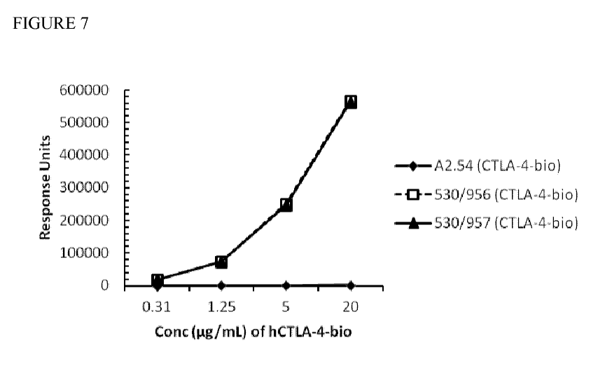

A dual ELISA was developed to assess simultaneous binding of the polypeptides produced in Example 5 to both CD40 and CTLA-4. In short, 96-well flat bottom plate high binding plates (Greiner #655074) were coated with CD40-Fc (Ancell # 504-820) by incubating overnight at 4° C. The plates were washed (Wash buffer: PBS+0.05% Tween 20 (PBST) Medicago #09-9410-100) and then blocked in PBST+3% BSA (Merck, #1.12018.0100). The plates were washed again and samples (957/530; 956/530) or control (0.2 ug/ml) were added to the wells. Mono-specific anti-CD40 antibody was used as the control (A2-54). The samples were incubated for 1 h at room temperature and then washed. Then biotinylated CTLA4-Fc (Orencia, Bristol-Mayers Squibb) (serially diluted 1/4 from 3.4-215 nM) was added. Streptavidin-HRP (Pierce #21126) was then added and the plates were subsequently developed using SuperSignal Pico Chemiluminescent substrate (Thermo #37069) and detected with Envision reader (Perkin Elmer). Results are shown in FIG. 7 and demonstrate that the bispecific molecules bind to the two antigens simultaneously.

### Surface Plasmon Resonance Analysis

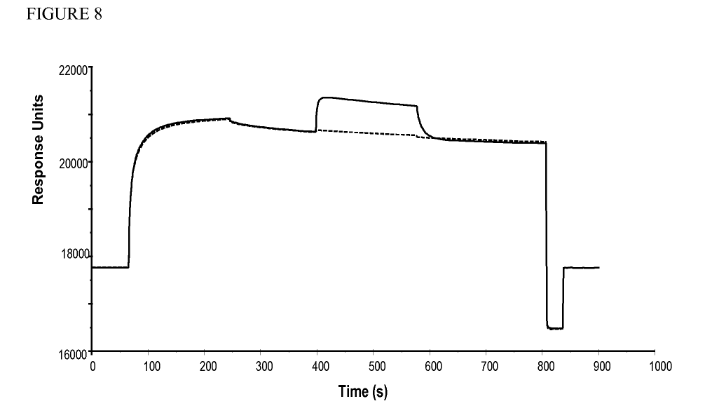

The ability of the bispecifc molecule to bind simulataneously to CTLA4 and CD40 was further confirmed by Biacore™ analysis. CD40-Fc (R&D #1493-CD) was immobilized to the BIAcore sensorship, CMS, using conventional amine coupling. 0.5 uM Bispecific construct (957/530) was injected over immobilized CD40 for 3 minutes and the dissociation was followed for 3 minutes. 2.17 uM CTLA4-Fc (Orencia, Bristol-Mayers Squibb) was injected over the CD40-Bispecific complex for 3 minutes and the dissociation phase was followed for 3 minutes. The chip was finally regenerated using 10 mM Glycine, pH 1.7. A Biacore 3000 instrument was used according to the manufacturer's protocols. Results are shown in FIG. 8. The black line shows binding of the bispecific molecule 957/530 to CD40 (time 75 s), and, upon injection of soluble CTLA-4 (time 400 s), simultaneous binding to CD40 and CTLA-4. The dashed line shows the binding of the control Ab, a monospecific antibody (530/531, A2-54) that only binds to CD40. The results demonstrate that the bi-specific molecule is able to simultaneously bind specifically to CD40 and to CTLA-4.

### Example 8—Demonstration of Dual Binding Specificity of Exemplary Bispecific Molecules to Targets Expressed by Cells

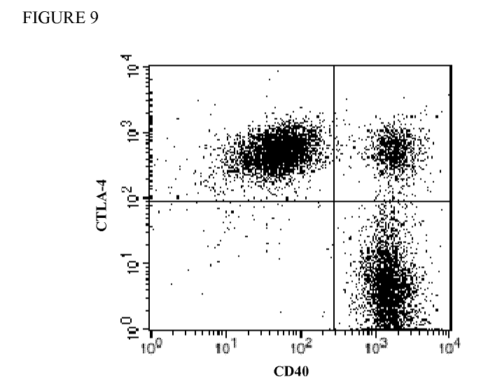

The ability of the bispecific antibodies to simultaneously bind to human CD40 expressed on WEHI cells and to human CTLA-4 expressed on HEK cells was determined in vitro by flow cytometry analysis. In short, WEHI-CD40 cells were labeled with PKH67 (Sigma-Aldrich # MIDI67) and HEK-CTLA4 cells were labeled with PKH26 (Sigma-Aldrich # MINI26), the cells were washed with PBS-2% BSA. 0.5×10⁶ per cell type were mixed with bispecific polypeptide (957/530) for 30 minutes at room temperature. The cells were washed and analyzed on a FACScalibur instrument, according to the manufacturer's instruction (Becton Dickinson, USA) and then the mean fluorescence intensity (“MFI”) was determined. A representative FACS plot is shown in FIG. 9. This confirms that simultaneous binding to the two targets by the bispecific polypeptide occurs in vitro, and that this bispecific binding results in cell-cell contact between CTLA-4 expressing cells and CD40 expressing cells. This was further confirmed by Fluorescence Microscopy (not shown).

### Example 9—Demonstration of the Ability of Exemplary Bispecific Molecules to Induce Proliferation in Human B Cells

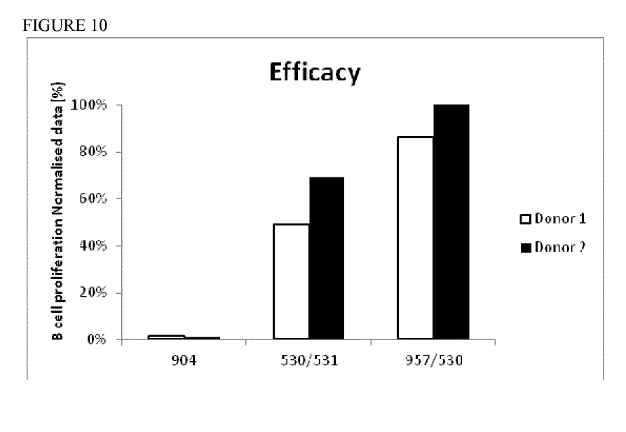

Buffy coats from peripheral blood were obtained from healthy blood donors at Uppsala University Hospital Blood Center and Lund University Hospital in Sweden. Human PBMCs were isolated by Ficoll-Paque density gradient centrifugation. CD19+B cells were isolated with MACS microbeads (Miltenyi Biotec) according to manufacturer's instructions. The CD19+cells (5×10⁴/well, >95% purity) were cultured in R10 medium with 10 ng/ml of IL-4 and dilution series of either a polypeptide of the invention (957/530), or a monospecific antibody for CD40 (530/531: A2-54), or the isolated polypeptide of amino acid sequence SEQ ID NO: 8 (soluble CTLA-4 binding domain reference 904) was used as a control as it binds specifically to only CTLA-4. After 48 to 72 h, the metabolic activity was measured with CellTiter-Glo (Promega, Wis., USA). The EC50 values were calculated using GraphPad Prism (GraphPad Software, Inc.). The results are shown in FIG. 10. The bispecific polypeptide of the invention produces a synergistic increase in proliferation in human B cells.

### Example 10—Cross-Reactivity to Cynomolgus CD40

The ability of exemplary bispecific polypeptides to bind to cells expressing cynomolgus CD40 was evaluated by measuring binding to HEK cells (ECACC, Salisbury, UK), transfected with the genes encoding the extracellular part of cynomolgus CD40. These HEK cells express cynomolgus CD40 on their surface. The concentrations of the bispecific polypeptides were 1 and 10 μg/ml. All 5 of the bispecific polypeptides tested bound to cynomolgus monkey CD40. The results are shown in the table below.

| | Cynomolgus |
|---|---|
| | monkey cros |
| Bispecific Polypeptide | reactivity |
| 1107/1145 (SEQ 110 with 1107 Heavy chain) | + |
| 1132/1139 (SEQ 112 with 1132 Heavy chain) | + |
| 1140/1141 (SEQ 113 with 1140 Heavy chain) | + |
| 1150/1152 (SEQ 114 with 1150 Heavy chain) | + |
| 1134/1141 (SEQ 113 with 1134 Heavy chain) | + |

### Example 11—Cross Reactivity to Murine CTLA-4

The relative affinity for murine and human CTLA-4 of an exemplary bispecific polypeptide of the invention was investigated using an inhibition ELISA binding assay. The bispecific polypeptide tested was 1140/1141 (SEQ 113 assembled with 1140 heavy chain). The CTLA-4-binding part of this polypeptide is the 1040 mutant CD86 molecule. The CTLA-4 binding properties of this CD86 molecule are not affected by conjugation to antibody.

In brief, 96-well flat bottom plate high binding plates (Greiner #655074) were coated with human CTLA-4 (Fitzgerald) incubating overnight at 4° C. The plates were washed (Wash buffer: PBS+0.05% Tween 20 (PBST) Medicago #09-9410-100) and then blocked in PBST+3% BSA (Merck, #1.12018.0100).

The sample (exemplary polypeptide) was pre-incubated at room temperature for 1 hour with soluble biotinylated human CTLA4 (Fitzgerald #30R-CD152) or soluble murine CTLA-4 (R&D systems) at different concentrations (serial dilutions 1/4 from 30000 to 0.3 ng/ml).

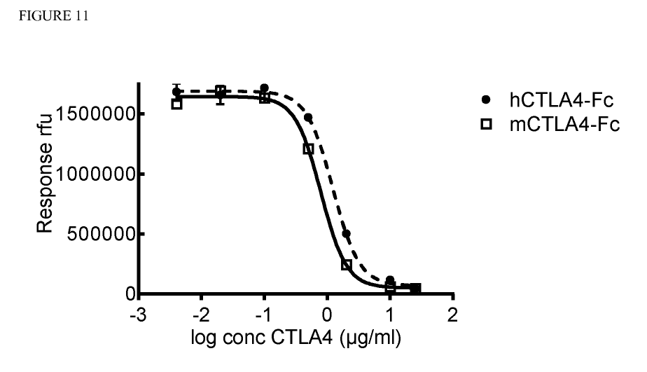

The mixture was then added to the blocked wells in the ELISA plate. Detection was performed with Streptavidin-HRP (Pierce #21126) and the plates were subsequently developed using SuperSignal Pico Chemiluminescent substrate (Thermo #37069) and detected with Envision reader (Perkin Elmer). The results are shown in FIG. 11. The observed inhibition curves with murine and human CTLA-4 demonstrate that the binding affinity of the exemplary polypeptide to the two forms of CTLA-4 is of a similar magnitude.

### Example 12—Binding to Human CD40

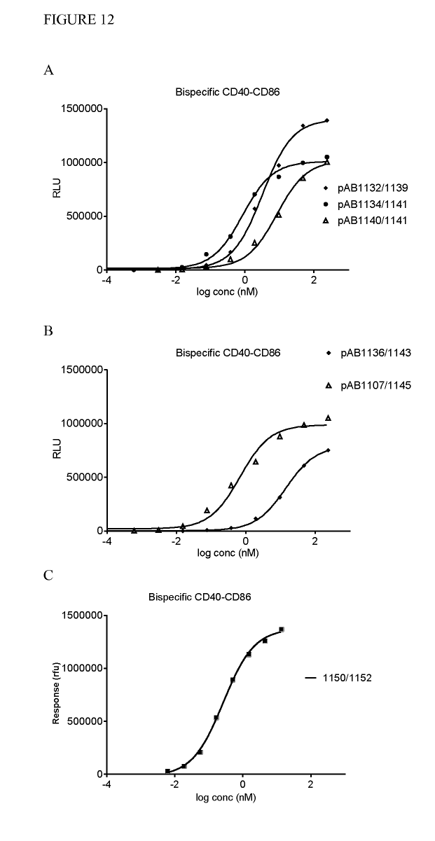

The ability of different exemplary bispecific polypeptides to bind to human CD40 was assayed by standard binding ELISA. The protocol was as described in Example 7 above. Results are shown as dose response curves in FIGS. 12A, B and C. The EC50 values ranged from 0.3 to 14 nM. The tested bispecific polypeptides include 1107/1145 (dimer of SEQ 110 with 1107 Heavy chain), 1136/1143 (dimer of SEQ 111 with 1143 Heavy chain), 1132/1139 (dimer of SEQ 112 with 1132 Heavy chain), 1140/1141 (dimer of SEQ 113 with 1140 Heavy chain), 1150/1152 (dimer of SEQ 114 with 1150 Heavy chain) and 1134/1141 (dimer SEQ 113 with 1134 Heavy chain).

### Example 13—Binding to Human CTLA-4

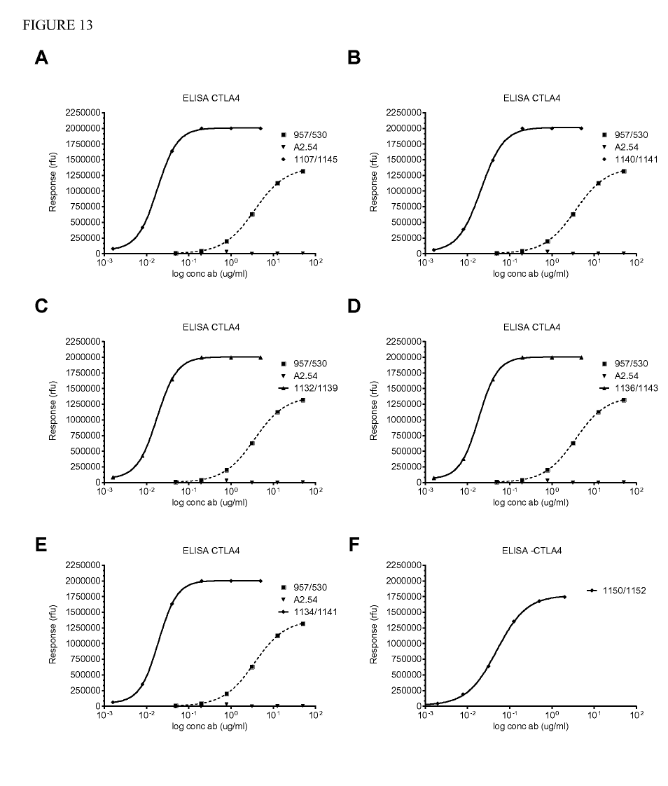

Six different exemplary bispecific polypeptides were tested for their ability to bind to human CD40 using standard binding ELISA. The protocol was as described in Example 7 above. Results are shown as dose response curves in FIGS. 13A to F. An exemplary bispecific molecule from Example 7 (957/530) was included for comparison, as well as the anti-CD40 mono-specific antibody, A2-54 as a negative control. All six tested polypeptides had an EC50 value calculated to be in the range approximately 0.02-0.05 μg/ml. Since all six share the same CD86 variant (1040), this indicates that different antibody conjugates do not affect the ability of this variant to bind CTLA-4. The tested bispecific polypeptides were 1107/1145 (dimer of SEQ 110 with 1107 Heavy chain), 1136/1143 (dimer of SEQ 111 with 1143 Heavy chain), 1132/1139 (dimer of SEQ 112 with 1132 Heavy chain), 1140/1141 (dimer of SEQ 113 with 1140 Heavy chain), 1150/1152 (dimer of SEQ 114 with 1150 Heavy chain) and 1134/1141 (dimer SEQ 113 with 1134 Heavy chain).

### Example 14—Dual Binding to CD40 and CTLA-4

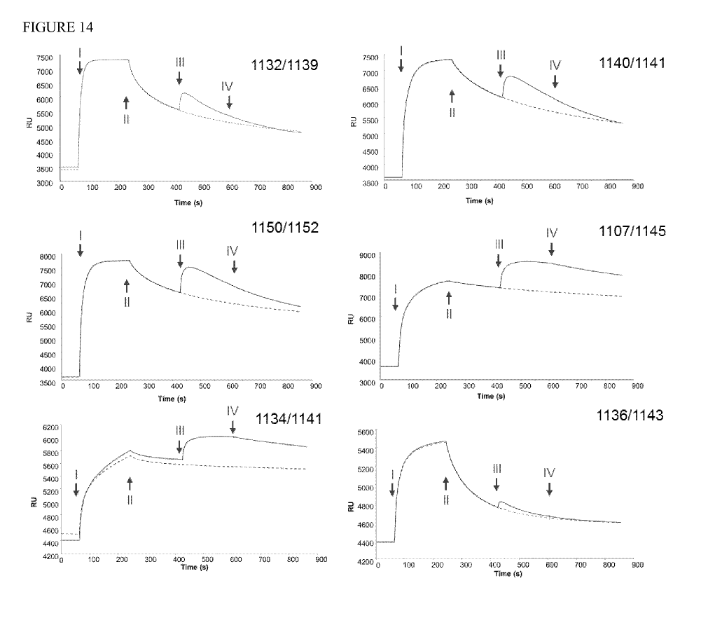

The six different exemplary bispecific polypeptides from Example 13 were tested for their ability to bind to both CD40 and CTLA-4. This was assessed by Surface Plasmon Resonance using the same protocol as in Example 7 above. The results are shown in FIG. 14. The kinetic parameters and the affinity constants were calculated using BlAevaluation 4.1 software and are shown in the tables below.

Affinity constants for binding to human CD40 as determined by Surface Plamson Resonance (Biacore).

| polypeptide | ka (1/Ms) | kd (1/s) | KD (M) |
|---|---|---|---|
| 1107/1145 | 3.47E+05 | 1.17E−04 | 3.36E−09 |
| 1132/1139 | 1.36E+06 | 8.44E−03 | 6.20E−09 |
| 1140/1141 | 4.70E+05 | 3.98E−03 | 8.47E−09 |
| 1150/1152 | 1.28E+06 | 3.26E−03 | 2.54E−09 |
| 1134/1141 | 1.34E+05 | 1.89E−03 | 1.41E−08 |
| 1136/1143 | 1.45E+05 | 5.63E−03 | 3.89E−08 |

Affinity constants for binding to human CTLA-4 as determined by Surface Plamson Resonance (Biacore).

| polypeptide | ka (1/Ms) | kd (1/s) | KD (M) |
|---|---|---|---|
| 1107/1145 | 1.02E+06 | 3.10E−03 | 3.05E−09 |
| 1132/1139 | 1.06E+06 | 3.11E−03 | 2.92E−09 |
| 1140/1141 | 7.97E+05 | 3.15E−03 | 3.95E−09 |
| 1150/1152 | 7.65E+05 | 3.12E−03 | 4.07E−09 |

### Example 15—Bispecific Polypeptide has an Anti-Tumor Effect In Vivo

The anti-tumor effect of exemplary bispecific polypeptide 1140/1141 (dimer of SEQ 113 with 1140 Heavy chain) was studied by administering the polypeptide to human CD40 transgenic mice inoculated with Murine bladder transitional cell carcinoma cancer cell line, Mouse Bladder-49 (MB49) cells. The treatment produced a statistically significant anti-tumor efficacy.

The animal model used for this experiment was created by injection of 2.5×10⁵ MB49 cells in the flank of hCD40tg mice. Bispecific polypeptide was administrated peritumorally with doses of 200 μmol (42 μg) on days 7 and 10 after inoculation, and tumor measurements were taken by caliper. The tumor growth and survival was followed over time.

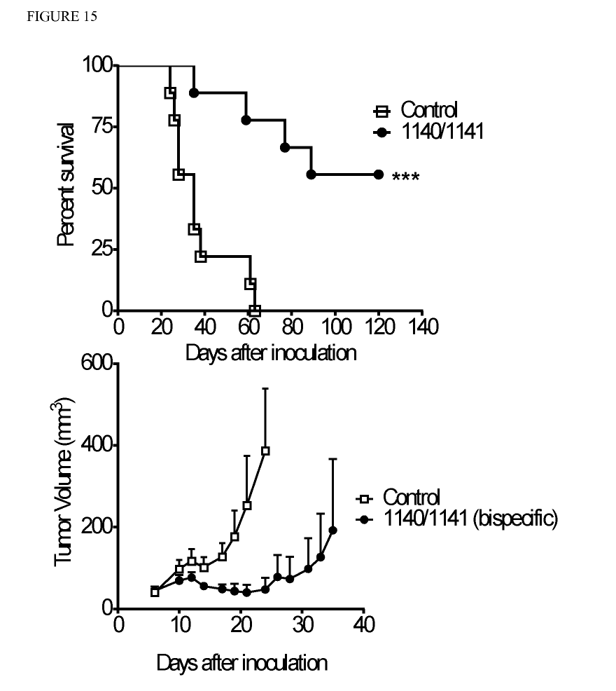

The treatment resulted in a significant anti-tumor efficacy, with improved survival and reduced tumour volume, as shown in FIGS. 15A and B.

### Example 16—Bispecific Polypeptide Establishes Anti-Tumour Immunological Memory In Vivo

Mice previously treated for bladder cancer and cured with the bispecific polypeptide 1140/1141 were re-challenged with bladder cancer cells. The treatment with the bispecific antibody clone was shown to have established an immunological memory for bladder cancer and hence immunity to tumors when the animals were re-challenged.

For this experiment, MB49 re-challenge was performed by injection of 2.5×10⁵ cells in the flank of hCD40tg mice that had previously been cured of MB49 tumors by treatment with 1140/1141 (as in Example 15). Naïve (i.e. not previously treated with polypeptide or inoculated) hCD40tg mice were used as controls. The tumor growth taken by caliper and survival was followed over time.

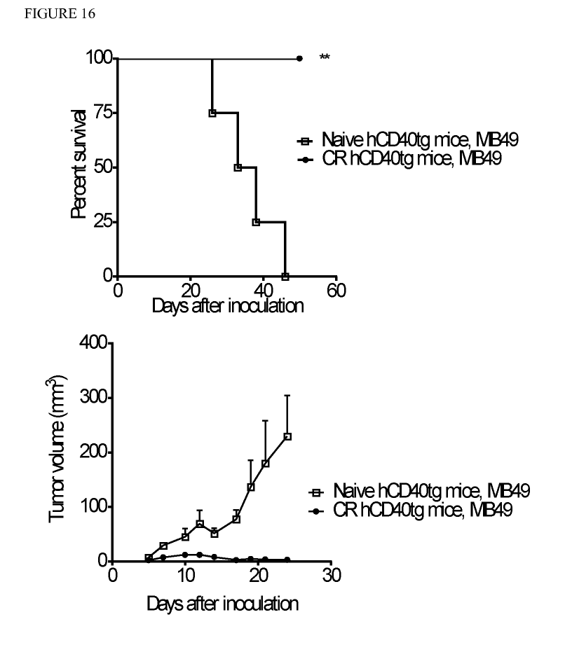

As shown in FIGS. 16A and B, the previously treated, re-challenged mice had 100% survival and the tumors regressed completely. Thus, the data shows that treatment with the bispecific polypeptide induces statistically significant immunity to tumor re-challenge with MB49 in hCD40tg mice. This demonstrates the presence of immunological memory. This result is of clinical relevance, since such anti-tumour immunological memory is necessary to establish a long lasting treatment effect particularly against metastatic tumors.

### Example 17—B Cell Proliferation Assay

The six different exemplary bispecific polypeptides from Example 13 were tested for their ability to stimulate human B cells. Each bispecific polypeptide was directly compared to the corresponding monospecific antibody lacking a CTLA-4 binding domain.

Human B cells from leukocyte filters were purified using RosetteSep Human B cell Enrichment Cocktail (Stemcell Technologies) and density separation using Ficoll-Paque Plus (GE Healthcare). B cell purity was assessed by staining with anti-human CD19-PE ab (Milteney). B cell purity varied between the different donors of the 3 experiments with a mean B cell purity of 69%.

B cells were seeded out on 96-wells plates together with different concentrations of the CD40-CD86 antibodies and human IL-4. The plates were incubated in a moisture chamber at 37C 5% C0² for 3 days. Metabolic activity was measured with Cell titer Glo (Promega) luminescence that measures the ATP content of the wells, which reflects B cell proliferation. S2C6 was used as a reference antibody. S2C6 is a murine anti-human CD40 antibody with agonistic effect in the B cell proliferation assay (Koho, Can Imm Imm 1984).

Dose-response curves of each antibody were plotted using graph Pad prism 4.0. The efficacy of each antibody was normalized by dividing the TOP value (as calculated using variable dose response curve fitting) of the antibody with the TOP value of the reference antibody S2C6.

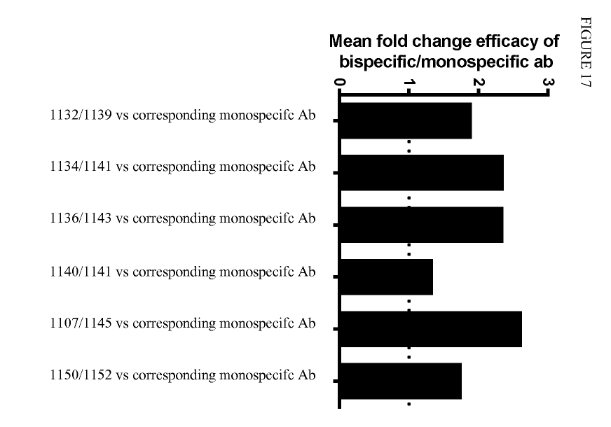

As shown in FIG. 17, the bispecific polypeptides displayed a higher efficacy in the B-cell proliferation assay than their corresponding monoclonal anti-CD40 antibody, indicating that the CTLA-4 binding part influences the ability to stimulate B-cell proliferation. This effect could be due to presence of cells (other than B cells) that express CD28 or CTLA-4 that may bind to the CD86-part of the antibody and thereby induce crosslinking of the antibody. The resulting increased immune activation has the potential to result in a stronger immune activation in vivo, generating an improved anti-tumor activity.

### Example 18—Activation of Immune Cells in Human PBMCs

Human PBMCs contain antigen presenting cells expressing CD40 and T cells expressing CTLA-4. Two of the exemplary bispecific molecules from Example 13 (1134/1141 and 1140/1141) were assayed for their ability to activate these immune cells. Each bispecific polypeptide was directly compared to the corresponding monospecific antibody lacking a CTLA-4 binding domain.

Human PBMCs from leukocyte filters or buffy blood (2-4 donors/experiment) were purified using density separation with Ficoll-Paque Plus (GE Healthcare). PBMCs were seeded out on CD3 pre-coated (overnight) 96-wells plates together with different concentrations of the bispecific polypeptide or the corresponding monospecific antibody. Staphylococcal enterotoxin A (SEA), a T cell stimulator, was added to the culture medium. The plates were incubated in a moisture chamber at 37C 5% C0² for 2 days and the IL-2 concentration in the supernatant was measured using a human IL-2 ELISA kit (BD Pharmingen). The IL-2 production at each tested concentration was calculated from the IL-2 ELISA standard curve.

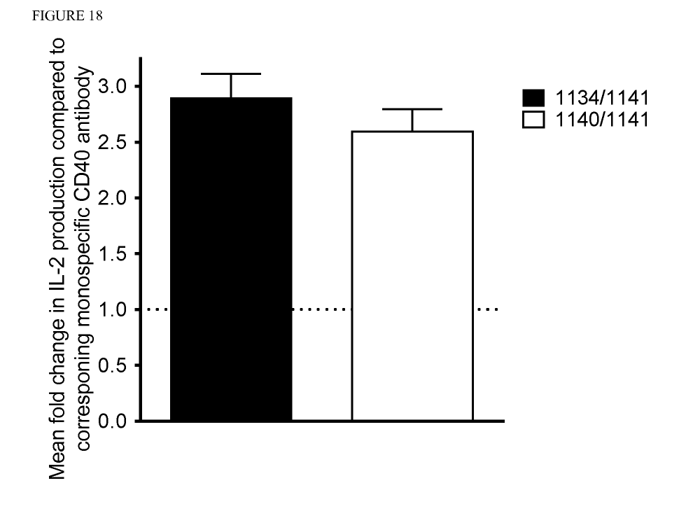

The mean fold change of each bispecific polypeptide relative to the corresponding monospecific antibody was calculated by dividing the mean of the two highest values (TOP value) of each bispecific polypeptide with the TOP value of the corresponding monospecific antibody. The two bispecific polypeptides generated a strong T cell activation as measured by IL-2 production. The mean fold change in IL-2 production was significantly higher relative to the monospecific antibodies, as shown in FIG. 18.

It has been determined (data not shown) that the anti-CD40 antibody portion of bispecific polypeptides 1134/1141 and 1140/1141 is not capable of blocking CD40L binding to the CD40 receptor. These anti-CD40 antibodies (and any which bind to the same epitope on CD40) thus have particularly favorable properties for use in a bispecific format with a CTLA-4 binding domain. Binding to this particular epitope on CD40 in a bispecific format enables a surprisingly strong immune activation, which has the potential to generate a strong anti-tumor effect in vivo.

### Example 19—CTLA-4 Induced Activation of B Cells

Immune activation was further investiaged by incubating cells transformed to express human CTLA-4 and B cells together with exemplary bispecific polypeptides.

2.5×10³ HEK wild type cells (HEK-wt) or HEK cells transduced to produce CTLA-4 (CTLA4-HEK) were seeded in the wells of a 96-well plate. Different concentrations of an exemplary bispecific polypeptide from Example 13 (1140/1141) were added to the cell containing wells and to empty wells, prior to pre-incubation in a moisture chamber at 37° C., 5% C02. After 2 h of incubation, 5×10³ B cells isolated from leukocyte filters of 2 healthy donors were added to each well together with IL-4 (10 ng/ml).

After 48 hours of incubation, cells were harvested and B cell activation was analyzed by co-staining of antibodies against anti-human CD19-PE (B cells) and the activation marker: anti-human CD83-APC (BD Biosciences). The mean fluorescence intensity (MFI) of CD83 expression was measured by flow cytometry on a FACSVerse, the data was analyzed using FlowJo software and statistical analysis was performed using Graphpad Prism 4.

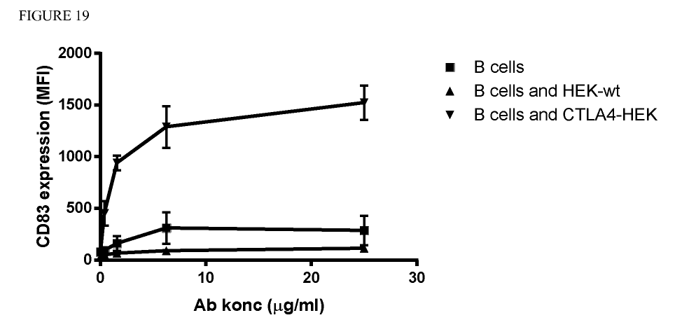

As shown in FIG. 19, the efficacy of the up-regulation of CD83 was increased 4-14 fold upon co-culturing of the bispecific polypeptide with CTLA4-HEK and B cells as compared to controls. The controls were either co-culturing of the bispecific polypeptide with HEK-wt and B cells, or the B cells alone.

It is known that many CD40 agonists require cross-linking via the Fc part of the antibody to induce optimal activity (Li et al 20011, Science, White et al 2011 J Immunol)). The CTLA-4 binding part of the bispecific polypeptide may provide this increase in cross-linking resulting in increased immune activation. The increased immune activation may result in increased anti-tumor effects in vivo.

1. A polypeptide capable of specifically binding to both CTLA-4 and CD40, said polypeptide comprising B1 and B2, wherein:
B1 is an antibody, or antigen binding fragment thereof, specific for CD40; and
B2 is a polypeptide binding domain specific for CTLA-4, which comprises or consists of:
i) the amino acid sequence of SEQ ID NO: 3; or
ii) an amino acid sequence in which at least one amino acid is changed when compared to the amino acid sequence of SEQ ID NO: 3 provided that said binding domain binds to human CTLA-4 with higher affinity than wild-type human CD86.

2. The polypeptide according to claim 1 in which the CTLA-4 specifically bound by the polypeptide is primate or murine, preferably human, CTLA-4, and/or wherein the CD40 specifically bound by the polypeptide is primate or murine, preferably human, CD40.

3. The polypeptide according to claim 1 in which B1 comprises at least one heavy chain (H) and/or at least one light chain (L) and B2 is attached to said at least one heavy chain (H) or least one light chain (L).

4. The polypeptide according to claim 3 in which B1 comprises:
at least one heavy chain (H) and at least one light chain (L) and B2 is attached to either the heavy chain or the light chain; or
two identical heavy chains (H) and two identical light chains (L) and B2 is attached to both heavy chains or to both light chains.

5. The polypeptide according to claim 1 which comprises a polypeptide arranged according to the following formula, written in the direction N-C:
H-(X)n-B2; (a)
B2-(X)n-H; (b)
L-(X)n-B2; or (c)
B2-(X)n-L (d)
wherein X is a linker and n is 0 or 1,
or a polypeptide which consists of a polypeptide arranged according to any one of formulae (a) to (d).

6. The polypeptide according to claim 5, wherein X is a peptide with the amino acid sequence SGGGGSGGGGS, SGGGGSGGGGSAP, NFSQP, KRTVA or (SG)m, where m=1 to 7.

7. The polypeptide according to claim 1, wherein 1, 2, 3, 4, 5, 6, 7, 8, 9 or 10 amino acids in said amino acid sequence of B2 (ii) are substituted when compared to the amino acid sequence of SEQ ID NO: 3; optionally wherein there are no insertions or deletions compared to the amino acid sequence of SEQ ID NO: 3.

8. The polypeptide according to claim 7, wherein at least one of said amino acid substitutions in said amino acid sequence of said first binding domain is at position 122, optionally wherein said amino acid sequence is also substituted in one or more of positions 32, 48, 49, 54, 74, 77, 79, 103, 107, 111, 118, 120, 121, 125, 127 and 134.

9-10. (canceled)

11. The polypeptide according to claim 8, wherein the substitutions in each position are selected from the following: F32I, Q48L, S49T, V541, V64/I, K74I/R, S77A, H79D/S/A, K103E, L107I/F/R, I111V, T118S, M120L, I121V, R122K/N, Q125E, N127S/D and A134T.

12. The polypeptide according to claim 1 wherein said amino acid sequence of B2 comprises or consists of an amino acid sequence selected from any one of SEQ ID NOs 8, 6, 7 and 9 to 24.

13. The polypeptide according to claim 1, wherein B1 comprises the heavy chain CDR sequences of an antibody as shown in Table A and/or the light chain CDR sequences of an antibody as shown in Table A, optionally wherein if both are present said heavy chain CDR sequences and said light chain CDR sequences are from the same antibody.

14. The polypeptide according to claim 1, wherein B1 comprises a variable heavy chain sequence of an antibody as shown in Table A and/or a variable light chain sequence of any antibody as shown in Table A, optionally wherein if both are present said variable heavy chain sequence and said variable light chain sequence are from the same antibody.

15. The polypeptide according to claim 1, wherein B1 comprises an human Fc region or a variant of a said region, where the region is an IgG1, IgG2, IgG3 or IgG4 region, preferably an IgG1 or IgG4 region.

16. The polypeptide according to claim 1, wherein B1 comprises a heavy chain sequence of an antibody as shown in Table A and/or a light chain sequence of an antibody as shown in Table A, optionally wherein if both are present said heavy chain sequence and said light chain sequence are from the same antibody.

17. The polypeptide according to claim 1, which comprises or consists of the amino acid sequence of any one of SEQ ID NOs: 56 to 60 or 110 to 114.

18. The polypeptide according to claim 1, which comprises or consists of the amino acid sequence of any one of SEQ ID NOs: 56 to 60 or 110 to 114, optionally wherein said polypeptide is a provided as a component part of an antibody.

19. (canceled)

20. A method of treating or preventing a disease or condition in an individual, the method comprising administering to an individual a polypeptide capable of specifically binding to both CTLA-4 and CD40, said polypeptide comprising B1 and B2, wherein:
B1 is an antibody, or antigen binding fragment thereof, specific for CD40; and
B2 is a polypeptide binding domain specific for CTLA-4, which comprises or consists of:
i) the amino acid sequence of SEQ ID NO: 3; or
ii) an amino acid sequence in which at least one amino acid is changed when compared to the amino acid sequence of SEQ ID NO: 3 provided that said binding domain binds to human CTLA-4 with higher affinity than wild-type human CD86.

21. The method according to claim 20, wherein the disease or condition is cancer and optionally wherein the individual is human.

22. The method according to claim 21, wherein the method comprises administering the polypeptide systemically or locally, such as at the site of a tumour or into a tumour draining lymph node.

23. The method according to claim 21 wherein the cancer is prostate cancer, breast cancer, colorectal cancer, pancreatic cancer, ovarian cancer, lung cancer, cervical cancer, rhabdomyosarcoma, neuroblastoma, multiple myeloma, leukemia, acute lymphoblastic leukemia, melanoma, bladder cancer, gastric cancer, head and neck cancer, liver cancer, skin cancer, lymphoma or glioblastoma.

24. (canceled)

25. The polypeptide according to claim 1 conjugated to an additional therapeutic moiety.

26. A composition comprising a polypeptide according to claim 1 and at least one pharmaceutically acceptable diluent or carrier.
